SEQUENCE LISTING (1248 sequences)

SEQ ID NO: 1 - RNA, 19 nt, Artificial Sequence
  Description of Artificial Sequence Synthetic oligonucleotide
uguucacuguucaaaacga

SEQ ID NO: 2 - RNA, 19 nt, Artificial Sequence
  Description of Artificial Sequence Synthetic oligonucleotide
ucguuuugaacagugaaca

SEQ ID NO: 3 - RNA, 19 nt, Artificial Sequence
  Description of Artificial Sequence Synthetic oligonucleotide
guucacuguucaaaacgaa

SEQ ID NO: 4 - RNA, 19 nt, Artificial Sequence
  Description of Artificial Sequence Synthetic oligonucleotide
uucguuuugaacagugaac

SEQ ID NO: 5 - RNA, 19 nt, Artificial Sequence
  Description of Artificial Sequence Synthetic oligonucleotide
uucacuguucaaaacgaag

SEQ ID NO: 6 - RNA, 19 nt, Artificial Sequence
  Description of Artificial Sequence Synthetic oligonucleotide
cuucguuuugaacagugaa

SEQ ID NO: 7 - RNA, 19 nt, Artificial Sequence
  Description of Artificial Sequence Synthetic oligonucleotide
ucauacgaaggauacuuuc

SEQ ID NO: 8 - RNA, 19 nt, Artificial Sequence
  Description of Artificial Sequence Synthetic oligonucleotide
gaaaguauccuucguauga

SEQ ID NO: 9 - RNA, 19 nt, Artificial Sequence
  Description of Artificial Sequence Synthetic oligonucleotide
cccaggacaugauaauaag

SEQ ID NO: 10 - RNA, 19 nt, Artificial Sequence
  Description of Artificial Sequence Synthetic oligonucleotide
cuuauuaucauguccuggg

SEQ ID NO: 11 - RNA, 19 nt, Artificial Sequence
  Description of Artificial Sequence Synthetic oligonucleotide
guucaaaacgaagacuagc

SEQ ID NO: 12 - RNA, 19 nt, Artificial Sequence
  Description of Artificial Sequence Synthetic oligonucleotide
gcuagucuucguuuugaac

SEQ ID NO: 13 - RNA, 19 nt, Artificial Sequence
  Description of Artificial Sequence Synthetic oligonucleotide
guauguauaaagauagcca

SEQ ID NO: 14 - RNA, 19 nt, Artificial Sequence
  Description of Artificial Sequence Synthetic oligonucleotide
uggcuaucuuuauacauac

SEQ ID NO: 15 - RNA, 19 nt, Artificial Sequence
  Description of Artificial Sequence Synthetic oligonucleotide
cauugaccaaggaaaucgg

SEQ ID NO: 16 - RNA, 19 nt, Artificial Sequence
  Description of Artificial Sequence Synthetic oligonucleotide
ccgauuuccuuggucaaug

SEQ ID NO: 17 - RNA, 19 nt, Artificial Sequence
  Description of Artificial Sequence Synthetic oligonucleotide
caaacuauuugucgcagga

SEQ ID NO: 18 - RNA, 19 nt, Artificial Sequence
  Description of Artificial Sequence Synthetic oligonucleotide
uccugcgacaaauaguuug

SEQ ID NO: 19 - RNA, 19 nt, Artificial Sequence
  Description of Artificial Sequence Synthetic oligonucleotide
acucucuccugugagaaca

SEQ ID NO: 20 - RNA, 19 nt, Artificial Sequence
  Description of Artificial Sequence Synthetic oligonucleotide
uguucucacaggagagagu

SEQ ID NO: 21 - RNA, 19 nt, Artificial Sequence
  Description of Artificial Sequence Synthetic oligonucleotide
uuuauugacaauacgcuuu

SEQ ID NO: 22 - RNA, 19 nt, Artificial Sequence
  Description of Artificial Sequence Synthetic oligonucleotide
aaagcguauugucaauaaa

SEQ ID NO: 23 - RNA, 19 nt, Artificial Sequence
  Description of Artificial Sequence Synthetic oligonucleotide
cuuccucucgcaacaaacu

SEQ ID NO: 24 - RNA, 19 nt, Artificial Sequence
  Description of Artificial Sequence Synthetic oligonucleotide
aguuuguugcgagaggaag

SEQ ID NO: 25 - RNA, 19 nt, Artificial Sequence
  Description of Artificial Sequence Synthetic oligonucleotide
gaccauauuuauuauaagu

SEQ ID NO: 26 - RNA, 19 nt, Artificial Sequence
  Description of Artificial Sequence Synthetic oligonucleotide
acuuauaauaaauaugguc

SEQ ID NO: 27 - RNA, 19 nt, Artificial Sequence
  Description of Artificial Sequence Synthetic oligonucleotide
ugaccaaggaaaucggccu

SEQ ID NO: 28 - RNA, 19 nt, Artificial Sequence
  Description of Artificial Sequence Synthetic oligonucleotide
aggccgauuuccuugguca

SEQ ID NO: 29 - RNA, 19 nt, Artificial Sequence
  Description of Artificial Sequence Synthetic oligonucleotide
ucaaaacgaagacuagcua

SEQ ID NO: 30 - RNA, 19 nt, Artificial Sequence
  Description of Artificial Sequence Synthetic oligonucleotide
uagcuagucuucguuuuga

SEQ ID NO: 31 - RNA, 19 nt, Artificial Sequence
  Description of Artificial Sequence Synthetic oligonucleotide
gaaucuucaucauacgaag

SEQ ID NO: 32 - RNA, 19 nt, Artificial Sequence
  Description of Artificial Sequence Synthetic oligonucleotide
cuucguaugaugaagauuc

SEQ ID NO: 33 - RNA, 19 nt, Artificial Sequence
  Description of Artificial Sequence Synthetic oligonucleotide
gggauagaucuauaauguu

SEQ ID NO: 34 - RNA, 19 nt, Artificial Sequence
  Description of Artificial Sequence Synthetic oligonucleotide
aacauuauagaucuauccc

SEQ ID NO: 35 - RNA, 19 nt, Artificial Sequence
  Description of Artificial Sequence Synthetic oligonucleotide
cuauuugucgcaggaauaa

SEQ ID NO: 36 - RNA, 19 nt, Artificial Sequence
  Description of Artificial Sequence Synthetic oligonucleotide
uuauuccugcgacaaauag

SEQ ID NO: 37 - RNA, 19 nt, Artificial Sequence
  Description of Artificial Sequence Synthetic oligonucleotide
ucuauuugaagauaugacu

SEQ ID NO: 38 - RNA, 19 nt, Artificial Sequence
  Description of Artificial Sequence Synthetic oligonucleotide
agucauaucuucaaauaga

SEQ ID NO: 39 - RNA, 19 nt, Artificial Sequence
  Description of Artificial Sequence Synthetic oligonucleotide
caacaaacuauuugucgca

SEQ ID NO: 40 - RNA, 19 nt, Artificial Sequence
  Description of Artificial Sequence Synthetic oligonucleotide
ugcgacaaauaguuuguug

SEQ ID NO: 41 - RNA, 19 nt, Artificial Sequence
  Description of Artificial Sequence Synthetic oligonucleotide
aucuucaucauacgaagga

SEQ ID NO: 42 - RNA, 19 nt, Artificial Sequence
  Description of Artificial Sequence Synthetic oligonucleotide
uccuucguaugaugaagau

SEQ ID NO: 43 - RNA, 19 nt, Artificial Sequence
  Description of Artificial Sequence Synthetic oligonucleotide
cucucgcaacaaacuauuu

SEQ ID NO: 44 - RNA, 19 nt, Artificial Sequence
  Description of Artificial Sequence Synthetic oligonucleotide
aaauaguuuguugcgagag

SEQ ID NO: 45 - RNA, 19 nt, Artificial Sequence
  Description of Artificial Sequence Synthetic oligonucleotide
ugaaucuucaucauacgaa

SEQ ID NO: 46 - RNA, 19 nt, Artificial Sequence
  Description of Artificial Sequence Synthetic oligonucleotide
uucguaugaugaagauuca

SEQ ID NO: 47 - RNA, 19 nt, Artificial Sequence
  Description of Artificial Sequence Synthetic oligonucleotide
cagaccuuccagaucgcuu

SEQ ID NO: 48 - RNA, 19 nt, Artificial Sequence
  Description of Artificial Sequence Synthetic oligonucleotide
aagcgaucuggaaggucug

SEQ ID NO: 49 - RNA, 19 nt, Artificial Sequence
  Description of Artificial Sequence Synthetic oligonucleotide
cagaucgcuuccucucgca

SEQ ID NO: 50 - RNA, 19 nt, Artificial Sequence
  Description of Artificial Sequence Synthetic oligonucleotide
ugcgagaggaagcgaucug

SEQ ID NO: 51 - RNA, 19 nt, Artificial Sequence
  Description of Artificial Sequence Synthetic oligonucleotide
cuagagguauggcuguaac

SEQ ID NO: 52 - RNA, 19 nt, Artificial Sequence
  Description of Artificial Sequence Synthetic oligonucleotide
guuacagccauaccucuag

SEQ ID NO: 53 - RNA, 19 nt, Artificial Sequence
  Description of Artificial Sequence Synthetic oligonucleotide
auuuauugacaauacgcuu

SEQ ID NO: 54 - RNA, 19 nt, Artificial Sequence
  Description of Artificial Sequence Synthetic oligonucleotide
aagcguauugucaauaaau

SEQ ID NO: 55 - RNA, 19 nt, Artificial Sequence
  Description of Artificial Sequence Synthetic oligonucleotide
acaauacgcuuuacuuuau

SEQ ID NO: 56 - RNA, 19 nt, Artificial Sequence
  Description of Artificial Sequence Synthetic oligonucleotide
auaaaguaaagcguauugu

SEQ ID NO: 57 - RNA, 19 nt, Artificial Sequence
  Description of Artificial Sequence Synthetic oligonucleotide
acgaaggauacuuucuagc

SEQ ID NO: 58 - RNA, 19 nt, Artificial Sequence
  Description of Artificial Sequence Synthetic oligonucleotide
gcuagaaaguauccuucgu

SEQ ID NO: 59 - RNA, 19 nt, Artificial Sequence
  Description of Artificial Sequence Synthetic oligonucleotide
ggaauugucucccagugca

SEQ ID NO: 60 - RNA, 19 nt, Artificial Sequence
  Description of Artificial Sequence Synthetic oligonucleotide
ugcacugggagacaauucc

SEQ ID NO: 61 - RNA, 19 nt, Artificial Sequence
  Description of Artificial Sequence Synthetic oligonucleotide
cagucuacacagcuucggg

SEQ ID NO: 62 - RNA, 19 nt, Artificial Sequence
  Description of Artificial Sequence Synthetic oligonucleotide
cccgaagcuguguagacug

SEQ ID NO: 63 - RNA, 19 nt, Artificial Sequence
  Description of Artificial Sequence Synthetic oligonucleotide
aucauacgaaggauacuuu

SEQ ID NO: 64 - RNA, 19 nt, Artificial Sequence
  Description of Artificial Sequence Synthetic oligonucleotide
aaaguauccuucguaugau

SEQ ID NO: 65 - RNA, 19 nt, Artificial Sequence
  Description of Artificial Sequence Synthetic oligonucleotide
uugaaucuucaucauacga

SEQ ID NO: 66 - RNA, 19 nt, Artificial Sequence
  Description of Artificial Sequence Synthetic oligonucleotide
ucguaugaugaagauucaa

SEQ ID NO: 67 - RNA, 19 nt, Artificial Sequence
  Description of Artificial Sequence Synthetic oligonucleotide
uuugaaucuucaucauacg

SEQ ID NO: 68 - RNA, 19 nt, Artificial Sequence
  Description of Artificial Sequence Synthetic oligonucleotide
cguaugaugaagauucaaa

SEQ ID NO: 69 - RNA, 19 nt, Artificial Sequence
  Description of Artificial Sequence Synthetic oligonucleotide
gaucgcuuccucucgcaac

SEQ ID NO: 70 - RNA, 19 nt, Artificial Sequence
  Description of Artificial Sequence Synthetic oligonucleotide
guugcgagaggaagcgauc

SEQ ID NO: 71 - RNA, 19 nt, Artificial Sequence
  Description of Artificial Sequence Synthetic oligonucleotide
gagguauggcuguaacuau

SEQ ID NO: 72 - RNA, 19 nt, Artificial Sequence
  Description of Artificial Sequence Synthetic oligonucleotide
auaguuacagccauaccuc

SEQ ID NO: 73 - RNA, 19 nt, Artificial Sequence
  Description of Artificial Sequence Synthetic oligonucleotide
ucccaggacaugauaauaa

SEQ ID NO: 74 - RNA, 19 nt, Artificial Sequence
  Description of Artificial Sequence Synthetic oligonucleotide
uuauuaucauguccuggga

SEQ ID NO: 75 - RNA, 19 nt, Artificial Sequence
  Description of Artificial Sequence Synthetic oligonucleotide
ucuacacagcuucgggaag

SEQ ID NO: 76 - RNA, 19 nt, Artificial Sequence
  Description of Artificial Sequence Synthetic oligonucleotide
cuucccgaagcuguguaga

SEQ ID NO: 77 - RNA, 19 nt, Artificial Sequence
  Description of Artificial Sequence Synthetic oligonucleotide
ucucgcaacaaacuauuug

SEQ ID NO: 78 - RNA, 19 nt, Artificial Sequence
  Description of Artificial Sequence Synthetic oligonucleotide
caaauaguuuguugcgaga

SEQ ID NO: 79 - RNA, 19 nt, Artificial Sequence
  Description of Artificial Sequence Synthetic oligonucleotide
gaaucacuugcacuccgga

SEQ ID NO: 80 - RNA, 19 nt, Artificial Sequence
  Description of Artificial Sequence Synthetic oligonucleotide
uccggagugcaagugauuc

SEQ ID NO: 81 - RNA, 19 nt, Artificial Sequence
  Description of Artificial Sequence Synthetic oligonucleotide
ugaaucuaaauuaucaguc

SEQ ID NO: 82 - RNA, 19 nt, Artificial Sequence
  Description of Artificial Sequence Synthetic oligonucleotide
gacugauaauuuagauuca

SEQ ID NO: 83 - RNA, 19 nt, Artificial Sequence
  Description of Artificial Sequence Synthetic oligonucleotide
gaauccuccugauaacauc

SEQ ID NO: 84 - RNA, 19 nt, Artificial Sequence
  Description of Artificial Sequence Synthetic oligonucleotide
gauguuaucaggaggauuc

SEQ ID NO: 85 - RNA, 19 nt, Artificial Sequence
  Description of Artificial Sequence Synthetic oligonucleotide
agagguauggcuguaacua

SEQ ID NO: 86 - RNA, 19 nt, Artificial Sequence
  Description of Artificial Sequence Synthetic oligonucleotide
uaguuacagccauaccucu

SEQ ID NO: 87 - RNA, 19 nt, Artificial Sequence
  Description of Artificial Sequence Synthetic oligonucleotide
gucccaggacaugauaaua

SEQ ID NO: 88 - RNA, 19 nt, Artificial Sequence
  Description of Artificial Sequence Synthetic oligonucleotide
uauuaucauguccugggac

SEQ ID NO: 89 - RNA, 19 nt, Artificial Sequence
  Description of Artificial Sequence Synthetic oligonucleotide
gauaacaucaaggauacaa

SEQ ID NO: 90 - RNA, 19 nt, Artificial Sequence
  Description of Artificial Sequence Synthetic oligonucleotide
uuguauccuugauguuauc

SEQ ID NO: 91 - RNA, 19 nt, Artificial Sequence
  Description of Artificial Sequence Synthetic oligonucleotide
acaaacuauuugucgcagg

SEQ ID NO: 92 - RNA, 19 nt, Artificial Sequence
  Description of Artificial Sequence Synthetic oligonucleotide
ccugcgacaaauaguuugu

SEQ ID NO: 93 - RNA, 19 nt, Artificial Sequence
  Description of Artificial Sequence Synthetic oligonucleotide
aaggauacuuucuagcuug

SEQ ID NO: 94 - RNA, 19 nt, Artificial Sequence
  Description of Artificial Sequence Synthetic oligonucleotide
caagcuagaaaguauccuu

SEQ ID NO: 95 - RNA, 19 nt, Artificial Sequence
  Description of Artificial Sequence Synthetic oligonucleotide
uauuaaaauuucaugccgg

SEQ ID NO: 96 - RNA, 19 nt, Artificial Sequence
  Description of Artificial Sequence Synthetic oligonucleotide
ccggcaugaaauuuuaaua

SEQ ID NO: 97 - RNA, 19 nt, Artificial Sequence
  Description of Artificial Sequence Synthetic oligonucleotide
uagccagccuagagguaug

SEQ ID NO: 98 - RNA, 19 nt, Artificial Sequence
  Description of Artificial Sequence Synthetic oligonucleotide
cauaccucuaggcuggcua

SEQ ID NO: 99 - RNA, 19 nt, Artificial Sequence
  Description of Artificial Sequence Synthetic oligonucleotide
uggcugcugaaccaguaga

SEQ ID NO: 100 - RNA, 19 nt, Artificial Sequence
  Description of Artificial Sequence Synthetic oligonucleotide
ucuacugguucagcagcca

SEQ ID NO: 101 - RNA, 19 nt, Artificial Sequence
  Description of Artificial Sequence Synthetic oligonucleotide
agaaucacuugcacuccgg

SEQ ID NO: 102 - RNA, 19 nt, Artificial Sequence
  Description of Artificial Sequence Synthetic oligonucleotide
ccggagugcaagugauucu

SEQ ID NO: 103 - RNA, 19 nt, Artificial Sequence
  Description of Artificial Sequence Synthetic oligonucleotide
auuaaaauuucaugccggg

SEQ ID NO: 104 - RNA, 19 nt, Artificial Sequence
  Description of Artificial Sequence Synthetic oligonucleotide
cccggcaugaaauuuuaau

SEQ ID NO: 105 - RNA, 19 nt, Artificial Sequence
  Description of Artificial Sequence Synthetic oligonucleotide
ugcagucuacacagcuucg

SEQ ID NO: 106 - RNA, 19 nt, Artificial Sequence
  Description of Artificial Sequence Synthetic oligonucleotide
cgaagcuguguagacugca

SEQ ID NO: 107 - RNA, 19 nt, Artificial Sequence
  Description of Artificial Sequence Synthetic oligonucleotide
gaucuauaauguucacugu

SEQ ID NO: 108 - RNA, 19 nt, Artificial Sequence
  Description of Artificial Sequence Synthetic oligonucleotide
acagugaacauuauagauc

SEQ ID NO: 109 - RNA, 19 nt, Artificial Sequence
  Description of Artificial Sequence Synthetic oligonucleotide
gcagucuacacagcuucgg

SEQ ID NO: 110 - RNA, 19 nt, Artificial Sequence
  Description of Artificial Sequence Synthetic oligonucleotide
ccgaagcuguguagacugc

SEQ ID NO: 111 - RNA, 19 nt, Artificial Sequence
  Description of Artificial Sequence Synthetic oligonucleotide
uuaaaauuucaugccgggc

SEQ ID NO: 112 - RNA, 19 nt, Artificial Sequence
  Description of Artificial Sequence Synthetic oligonucleotide
gcccggcaugaaauuuuaa

SEQ ID NO: 113 - RNA, 19 nt, Artificial Sequence
  Description of Artificial Sequence Synthetic oligonucleotide
aacaaacuauuugucgcag

SEQ ID NO: 114 - RNA, 19 nt, Artificial Sequence
  Description of Artificial Sequence Synthetic oligonucleotide
cugcgacaaauaguuuguu

SEQ ID NO: 115 - RNA, 19 nt, Artificial Sequence
  Description of Artificial Sequence Synthetic oligonucleotide
aauuuauugacaauacgcu

SEQ ID NO: 116 - RNA, 19 nt, Artificial Sequence
  Description of Artificial Sequence Synthetic oligonucleotide
agcguauugucaauaaauu

SEQ ID NO: 117 - RNA, 19 nt, Artificial Sequence
  Description of Artificial Sequence Synthetic oligonucleotide
aaaauuucaugccgggcgc

SEQ ID NO: 118 - RNA, 19 nt, Artificial Sequence
  Description of Artificial Sequence Synthetic oligonucleotide
gcgcccggcaugaaauuuu

SEQ ID NO: 119 - RNA, 19 nt, Artificial Sequence
  Description of Artificial Sequence Synthetic oligonucleotide
guguagugacgcaugcccu

SEQ ID NO: 120 - RNA, 19 nt, Artificial Sequence
  Description of Artificial Sequence Synthetic oligonucleotide
agggcaugcgucacuacac

SEQ ID NO: 121 - RNA, 19 nt, Artificial Sequence
  Description of Artificial Sequence Synthetic oligonucleotide
aaauuuauugacaauacgc

SEQ ID NO: 122 - RNA, 19 nt, Artificial Sequence
  Description of Artificial Sequence Synthetic oligonucleotide
gcguauugucaauaaauuu

SEQ ID NO: 123 - RNA, 19 nt, Artificial Sequence
  Description of Artificial Sequence Synthetic oligonucleotide
auugaccaaggaaaucggc

SEQ ID NO: 124 - RNA, 19 nt, Artificial Sequence
  Description of Artificial Sequence Synthetic oligonucleotide
gccgauuuccuuggucaau

SEQ ID NO: 125 - RNA, 19 nt, Artificial Sequence
  Description of Artificial Sequence Synthetic oligonucleotide
uguagugacgcaugcccuc

SEQ ID NO: 126 - RNA, 19 nt, Artificial Sequence
  Description of Artificial Sequence Synthetic oligonucleotide
gagggcaugcgucacuaca

SEQ ID NO: 127 - RNA, 19 nt, Artificial Sequence
  Description of Artificial Sequence Synthetic oligonucleotide
ccggaccauauuuauuaua

SEQ ID NO: 128 - RNA, 19 nt, Artificial Sequence
  Description of Artificial Sequence Synthetic oligonucleotide
uauaauaaauaugguccgg

SEQ ID NO: 129 - RNA, 19 nt, Artificial Sequence
  Description of Artificial Sequence Synthetic oligonucleotide
agccagccuagagguaugg

SEQ ID NO: 130 - RNA, 19 nt, Artificial Sequence
  Description of Artificial Sequence Synthetic oligonucleotide
ccauaccucuaggcuggcu

SEQ ID NO: 131 - RNA, 19 nt, Artificial Sequence
  Description of Artificial Sequence Synthetic oligonucleotide
guagugacgcaugcccuca

SEQ ID NO: 132 - RNA, 19 nt, Artificial Sequence
  Description of Artificial Sequence Synthetic oligonucleotide
ugagggcaugcgucacuac

SEQ ID NO: 133 - RNA, 19 nt, Artificial Sequence
  Description of Artificial Sequence Synthetic oligonucleotide
gacgcaugcccucaauccc

SEQ ID NO: 134 - RNA, 19 nt, Artificial Sequence
  Description of Artificial Sequence Synthetic oligonucleotide
gggauugagggcaugcguc

SEQ ID NO: 135 - RNA, 19 nt, Artificial Sequence
  Description of Artificial Sequence Synthetic oligonucleotide
aaucuucaucauacgaagg

SEQ ID NO: 136 - RNA, 19 nt, Artificial Sequence
  Description of Artificial Sequence Synthetic oligonucleotide
ccuucguaugaugaagauu

SEQ ID NO: 137 - RNA, 19 nt, Artificial Sequence
  Description of Artificial Sequence Synthetic oligonucleotide
ggaaaucggccucuauuug

SEQ ID NO: 138 - RNA, 19 nt, Artificial Sequence
  Description of Artificial Sequence Synthetic oligonucleotide
caaauagaggccgauuucc

SEQ ID NO: 139 - RNA, 19 nt, Artificial Sequence
  Description of Artificial Sequence Synthetic oligonucleotide
ucagaccuuccagaucgcu

SEQ ID NO: 140 - RNA, 19 nt, Artificial Sequence
  Description of Artificial Sequence Synthetic oligonucleotide
agcgaucuggaaggucuga

SEQ ID NO: 141 - RNA, 19 nt, Artificial Sequence
  Description of Artificial Sequence Synthetic oligonucleotide
auaugacugauucugacug

SEQ ID NO: 142 - RNA, 19 nt, Artificial Sequence
  Description of Artificial Sequence Synthetic oligonucleotide
cagucagaaucagucauau

SEQ ID NO: 143 - RNA, 19 nt, Artificial Sequence
  Description of Artificial Sequence Synthetic oligonucleotide
uugcacuccggagguagag

SEQ ID NO: 144 - RNA, 19 nt, Artificial Sequence
  Description of Artificial Sequence Synthetic oligonucleotide
cucuaccuccggagugcaa

SEQ ID NO: 145 - RNA, 19 nt, Artificial Sequence
  Description of Artificial Sequence Synthetic oligonucleotide
cuugcacuccggagguaga

SEQ ID NO: 146 - RNA, 19 nt, Artificial Sequence
  Description of Artificial Sequence Synthetic oligonucleotide
ucuaccuccggagugcaag

SEQ ID NO: 147 - RNA, 19 nt, Artificial Sequence
  Description of Artificial Sequence Synthetic oligonucleotide
ugacgcaugcccucaaucc

SEQ ID NO: 148 - RNA, 19 nt, Artificial Sequence
  Description of Artificial Sequence Synthetic oligonucleotide
ggauugagggcaugcguca

SEQ ID NO: 149 - DNA, 21 bp, Artificial Sequence
  Description of Artificial Sequence Synthetic oligonucleotide
uguucacuguucaaaacgatt

SEQ ID NO: 150 - DNA, 21 bp, Artificial Sequence
  Description of Artificial Sequence Synthetic oligonucleotide
ucguuuugaacagugaacatt

SEQ ID NO: 151 - DNA, 21 bp, Artificial Sequence
  Description of Artificial Sequence Synthetic oligonucleotide
uguucacuguucaaaacgatt

SEQ ID NO: 152 - DNA, 21 bp, Artificial Sequence
  Description of Artificial Sequence Synthetic oligonucleotide
ucguuuugaacagugaacatt

SEQ ID NO: 153 - DNA, 21 bp, Artificial Sequence
  Description of Artificial Sequence Synthetic oligonucleotide
uguucacuguucaaaacgatt

SEQ ID NO: 154 - DNA, 21 bp, Artificial Sequence
  Description of Artificial Sequence Synthetic oligonucleotide
ucguuuugaacagugaacatt

SEQ ID NO: 155 - DNA, 21 bp, Artificial Sequence
  Description of Artificial Sequence Synthetic oligonucleotide
uguucacuguucaaaacgatt

SEQ ID NO: 156 - DNA, 21 bp, Artificial Sequence
  Description of Artificial Sequence Synthetic oligonucleotide
ucguuuugaacagugaacatt

SEQ ID NO: 157 - DNA, 21 bp, Artificial Sequence
  Description of Artificial Sequence Synthetic oligonucleotide
uguucacuguucaaaacgatt

SEQ ID NO: 158 - DNA, 21 bp, Artificial Sequence
  Description of Artificial Sequence Synthetic oligonucleotide
ucguuuugaacagugaacatt

SEQ ID NO: 159 - DNA, 21 bp, Artificial Sequence
  Description of Artificial Sequence Synthetic oligonucleotide
tguucacuguucaaaacgatt

SEQ ID NO: 160 - DNA, 21 bp, Artificial Sequence
  Description of Artificial Sequence Synthetic oligonucleotide
ucguuuugaacagugaacatt

SEQ ID NO: 161 - DNA, 21 bp, Artificial Sequence
  Description of Artificial Sequence Synthetic oligonucleotide
tguucacuguucaaaacgatt

SEQ ID NO: 162 - DNA, 21 bp, Artificial Sequence
  Description of Artificial Sequence Synthetic oligonucleotide
ucguuuugaacagugaacatt

SEQ ID NO: 163 - DNA, 21 bp, Artificial Sequence
  Description of Artificial Sequence Synthetic oligonucleotide
tguucacuguucaaaacgatt

SEQ ID NO: 164 - DNA, 21 bp, Artificial Sequence
  Description of Artificial Sequence Synthetic oligonucleotide
ucguuuugaacagugaacatt

SEQ ID NO: 165 - DNA, 21 bp, Artificial Sequence
  Description of Artificial Sequence Synthetic oligonucleotide
tguucacuguucaaaacgatt

SEQ ID NO: 166 - DNA, 21 bp, Artificial Sequence
  Description of Artificial Sequence Synthetic oligonucleotide
ucguuuugaacagugaacatt

SEQ ID NO: 167 - DNA, 21 bp, Artificial Sequence
  Description of Artificial Sequence Synthetic oligonucleotide
tguucacuguucaaaacgatt

SEQ ID NO: 168 - DNA, 21 bp, Artificial Sequence
  Description of Artificial Sequence Synthetic oligonucleotide
ucguuuugaacagugaacatt

SEQ ID NO: 169 - DNA, 21 bp, Artificial Sequence
  Description of Artificial Sequence Synthetic oligonucleotide
uguucacuguucaaaacgatt

SEQ ID NO: 170 - DNA, 21 bp, Artificial Sequence
  Description of Artificial Sequence Synthetic oligonucleotide
ucguuuugaacagugaacatt

SEQ ID NO: 171 - DNA, 21 bp, Artificial Sequence
  Description of Artificial Sequence Synthetic oligonucleotide
uguucacuguucaaaacgatt

SEQ ID NO: 172 - DNA, 21 bp, Artificial Sequence
  Description of Artificial Sequence Synthetic oligonucleotide
ucguuuugaacagugaacatt

SEQ ID NO: 173 - DNA, 21 bp, Artificial Sequence
  Description of Artificial Sequence Synthetic oligonucleotide
uguucacuguucaaaacgatt

SEQ ID NO: 174 - DNA, 21 bp, Artificial Sequence
  Description of Artificial Sequence Synthetic oligonucleotide
ucguuuugaacagugaacatt

SEQ ID NO: 175 - DNA, 21 bp, Artificial Sequence
  Description of Artificial Sequence Synthetic oligonucleotide
uguucacuguucaaaacgatt

SEQ ID NO: 176 - DNA, 21 bp, Artificial Sequence
  Description of Artificial Sequence Synthetic oligonucleotide
ucguuuugaacagugaacatt

SEQ ID NO: 177 - DNA, 21 bp, Artificial Sequence
  Description of Artificial Sequence Synthetic oligonucleotide
uguucacuguucaaaacgatt

SEQ ID NO: 178 - DNA, 21 bp, Artificial Sequence
  Description of Artificial Sequence Synthetic oligonucleotide
ucguuuugaacagugaacatt

SEQ ID NO: 179 - DNA, 21 bp, Artificial Sequence
  Description of Artificial Sequence Synthetic oligonucleotide
guucacuguucaaaacgaatt

SEQ ID NO: 180 - DNA, 21 bp, Artificial Sequence
  Description of Artificial Sequence Synthetic oligonucleotide
uucguuuugaacagugaactt

SEQ ID NO: 181 - DNA, 21 bp, Artificial Sequence
  Description of Artificial Sequence Synthetic oligonucleotide
uucacuguucaaaacgaagtt

SEQ ID NO: 182 - DNA, 21 bp, Artificial Sequence
  Description of Artificial Sequence Synthetic oligonucleotide
cuucguuuugaacagugaatt

SEQ ID NO: 183 - DNA, 21 bp, Artificial Sequence
  Description of Artificial Sequence Synthetic oligonucleotide
ucauacgaaggauacuuuctt

SEQ ID NO: 184 - DNA, 21 bp, Artificial Sequence
  Description of Artificial Sequence Synthetic oligonucleotide
gaaaguauccuucguaugatt

SEQ ID NO: 185 - DNA, 21 bp, Artificial Sequence
  Description of Artificial Sequence Synthetic oligonucleotide
cccaggacaugauaauaagtt

SEQ ID NO: 186 - DNA, 21 bp, Artificial Sequence
  Description of Artificial Sequence Synthetic oligonucleotide
cuuauuaucauguccugggtt

SEQ ID NO: 187 - DNA, 22 bp, Artificial Sequence
  Description of Artificial Sequence Synthetic oligonucleotide
tcccaggacaugauaauaagtt

SEQ ID NO: 188 - DNA, 21 bp, Artificial Sequence
  Description of Artificial Sequence Synthetic oligonucleotide
cuuauuaucauguccugggtt

SEQ ID NO: 189 - DNA, 21 bp, Artificial Sequence
  Description of Artificial Sequence Synthetic oligonucleotide
guucaaaacgaagacuagctt

SEQ ID NO: 190 - DNA, 21 bp, Artificial Sequence
  Description of Artificial Sequence Synthetic oligonucleotide
gcuagucuucguuuugaactt

SEQ ID NO: 191 - DNA, 21 bp, Artificial Sequence
  Description of Artificial Sequence Synthetic oligonucleotide
guucaaaacgaagacuagctt

SEQ ID NO: 192 - DNA, 21 bp, Artificial Sequence
  Description of Artificial Sequence Synthetic oligonucleotide
gcuagucuucguuuugaactt

SEQ ID NO: 193 - DNA, 21 bp, Artificial Sequence
  Description of Artificial Sequence Synthetic oligonucleotide
guucaaaacgaagacuagctt

SEQ ID NO: 194 - DNA, 21 bp, Artificial Sequence
  Description of Artificial Sequence Synthetic oligonucleotide
gcuagucuucguuuugaactt

SEQ ID NO: 195 - DNA, 21 bp, Artificial Sequence
  Description of Artificial Sequence Synthetic oligonucleotide
guucaaaacgaagacuagctt

SEQ ID NO: 196 - DNA, 21 bp, Artificial Sequence
  Description of Artificial Sequence Synthetic oligonucleotide
gcuagucuucguuuugaactt

SEQ ID NO: 197 - DNA, 21 bp, Artificial Sequence
  Description of Artificial Sequence Synthetic oligonucleotide
tuucaaaacgaagacuagctt

SEQ ID NO: 198 - DNA, 21 bp, Artificial Sequence
  Description of Artificial Sequence Synthetic oligonucleotide
gcuagucuucguuuugaactt

SEQ ID NO: 199 - DNA, 21 bp, Artificial Sequence
  Description of Artificial Sequence Synthetic oligonucleotide
tuucaaaacgaagacuagctt

SEQ ID NO: 200 - DNA, 21 bp, Artificial Sequence
  Description of Artificial Sequence Synthetic oligonucleotide
gcuagucuucguuuugaactt

SEQ ID NO: 201 - DNA, 21 bp, Artificial Sequence
  Description of Artificial Sequence Synthetic oligonucleotide
tuucaaaacgaagacuagctt

SEQ ID NO: 202 - DNA, 21 bp, Artificial Sequence
  Description of Artificial Sequence Synthetic oligonucleotide
gcuagucuucguuuugaactt

SEQ ID NO: 203 - DNA, 21 bp, Artificial Sequence
  Description of Artificial Sequence Synthetic oligonucleotide
tuucaaaacgaagacuagctt

SEQ ID NO: 204 - DNA, 21 bp, Artificial Sequence
  Description of Artificial Sequence Synthetic oligonucleotide
gcuagucuucguuuugaactt

SEQ ID NO: 205 - DNA, 21 bp, Artificial Sequence
  Description of Artificial Sequence Synthetic oligonucleotide
guucaaaacgaagacuagctt

SEQ ID NO: 206 - DNA, 21 bp, Artificial Sequence
  Description of Artificial Sequence Synthetic oligonucleotide
gcuagucuucguuuugaactt

SEQ ID NO: 207 - DNA, 21 bp, Artificial Sequence
  Description of Artificial Sequence Synthetic oligonucleotide
guucaaaacgaagacuagctt

SEQ ID NO: 208 - DNA, 21 bp, Artificial Sequence
  Description of Artificial Sequence Synthetic oligonucleotide
gcuagucuucguuuugaactt

SEQ ID NO: 209 - DNA, 21 bp, Artificial Sequence
  Description of Artificial Sequence Synthetic oligonucleotide
guucaaaacgaagacuagctt

SEQ ID NO: 210 - DNA, 21 bp, Artificial Sequence
  Description of Artificial Sequence Synthetic oligonucleotide
gcuagucuucguuuugaactt

SEQ ID NO: 211 - DNA, 21 bp, Artificial Sequence
  Description of Artificial Sequence Synthetic oligonucleotide
guucaaaacgaagacuagctt

SEQ ID NO: 212 - DNA, 21 bp, Artificial Sequence
  Description of Artificial Sequence Synthetic oligonucleotide
gcuagucuucguuuugaactt

SEQ ID NO: 213 - DNA, 21 bp, Artificial Sequence
  Description of Artificial Sequence Synthetic oligonucleotide
guauguauaaagauagccatt

SEQ ID NO: 214 - DNA, 21 bp, Artificial Sequence
  Description of Artificial Sequence Synthetic oligonucleotide
uggcuaucuuuauacauactt

SEQ ID NO: 215 - DNA, 21 bp, Artificial Sequence
  Description of Artificial Sequence Synthetic oligonucleotide
cauugaccaaggaaaucggtt

SEQ ID NO: 216 - DNA, 21 bp, Artificial Sequence
  Description of Artificial Sequence Synthetic oligonucleotide
ccgauuuccuuggucaaugtt

SEQ ID NO: 217 - DNA, 21 bp, Artificial Sequence
  Description of Artificial Sequence Synthetic oligonucleotide
caaacuauuugucgcaggatt

SEQ ID NO: 218 - DNA, 21 bp, Artificial Sequence
  Description of Artificial Sequence Synthetic oligonucleotide
uccugcgacaaauaguuugtt

SEQ ID NO: 219 - DNA, 22 bp, Artificial Sequence
  Description of Artificial Sequence Synthetic oligonucleotide
tcaaacuauuugucgcaggatt

SEQ ID NO: 220 - DNA, 21 bp, Artificial Sequence
  Description of Artificial Sequence Synthetic oligonucleotide
uccugcgacaaauaguuugtt

SEQ ID NO: 221 - DNA, 21 bp, Artificial Sequence
  Description of Artificial Sequence Synthetic oligonucleotide
acucucuccugugagaacatt

SEQ ID NO: 222 - DNA, 21 bp, Artificial Sequence
  Description of Artificial Sequence Synthetic oligonucleotide
uguucucacaggagagagutt

SEQ ID NO: 223 - DNA, 21 bp, Artificial Sequence
  Description of Artificial Sequence Synthetic oligonucleotide
acucucuccugugagaacatt

SEQ ID NO: 224 - DNA, 21 bp, Artificial Sequence
  Description of Artificial Sequence Synthetic oligonucleotide
uguucucacaggagagagutt

SEQ ID NO: 225 - DNA, 21 bp, Artificial Sequence
  Description of Artificial Sequence Synthetic oligonucleotide
acucucuccugugagaacatt

SEQ ID NO: 226 - DNA, 21 bp, Artificial Sequence
  Description of Artificial Sequence Synthetic oligonucleotide
uguucucacaggagagagutt

SEQ ID NO: 227 - DNA, 21 bp, Artificial Sequence
  Description of Artificial Sequence Synthetic oligonucleotide
acucucuccugugagaacatt

SEQ ID NO: 228 - DNA, 21 bp, Artificial Sequence
  Description of Artificial Sequence Synthetic oligonucleotide
uguucucacaggagagagutt

SEQ ID NO: 229 - DNA, 21 bp, Artificial Sequence
  Description of Artificial Sequence Synthetic oligonucleotide
acucucuccugugagaacatt

SEQ ID NO: 230 - DNA, 21 bp, Artificial Sequence
  Description of Artificial Sequence Synthetic oligonucleotide
uguucucacaggagagagutt

SEQ ID NO: 231 - DNA, 21 bp, Artificial Sequence
  Description of Artificial Sequence Synthetic oligonucleotide
acucucuccugugagaacatt

SEQ ID NO: 232 - DNA, 21 bp, Artificial Sequence
  Description of Artificial Sequence Synthetic oligonucleotide
uguucucacaggagagagutt

SEQ ID NO: 233 - DNA, 21 bp, Artificial Sequence
  Description of Artificial Sequence Synthetic oligonucleotide
acucucuccugugagaacatt

SEQ ID NO: 234 - DNA, 21 bp, Artificial Sequence
  Description of Artificial Sequence Synthetic oligonucleotide
uguucucacaggagagagutt

SEQ ID NO: 235 - DNA, 21 bp, Artificial Sequence
  Description of Artificial Sequence Synthetic oligonucleotide
acucucuccugugagaacatt

SEQ ID NO: 236 - DNA, 21 bp, Artificial Sequence
  Description of Artificial Sequence Synthetic oligonucleotide
uguucucacaggagagagutt

SEQ ID NO: 237 - DNA, 21 bp, Artificial Sequence
  Description of Artificial Sequence Synthetic oligonucleotide
acucucuccugugagaacatt

SEQ ID NO: 238 - DNA, 21 bp, Artificial Sequence
  Description of Artificial Sequence Synthetic oligonucleotide
uguucucacaggagagagutt

SEQ ID NO: 239 - DNA, 21 bp, Artificial Sequence
  Description of Artificial Sequence Synthetic oligonucleotide
tcucucuccugugagaacatt

SEQ ID NO: 240 - DNA, 21 bp, Artificial Sequence
  Description of Artificial Sequence Synthetic oligonucleotide
uguucucacaggagagagutt

SEQ ID NO: 241 - DNA, 21 bp, Artificial Sequence
  Description of Artificial Sequence Synthetic oligonucleotide
tcucucuccugugagaacatt

SEQ ID NO: 242 - DNA, 21 bp, Artificial Sequence
  Description of Artificial Sequence Synthetic oligonucleotide
uguucucacaggagagagutt

SEQ ID NO: 243 - DNA, 21 bp, Artificial Sequence
  Description of Artificial Sequence Synthetic oligonucleotide
acucucuccugugagaacatt

SEQ ID NO: 244 - DNA, 21 bp, Artificial Sequence
  Description of Artificial Sequence Synthetic oligonucleotide
uguucucacaggagagagutt

SEQ ID NO: 245 - DNA, 21 bp, Artificial Sequence
  Description of Artificial Sequence Synthetic oligonucleotide
acucucuccugugagaacatt

SEQ ID NO: 246 - DNA, 21 bp, Artificial Sequence
  Description of Artificial Sequence Synthetic oligonucleotide
uguucucacaggagagagutt

SEQ ID NO: 247 - DNA, 21 bp, Artificial Sequence
  Description of Artificial Sequence Synthetic oligonucleotide
uuuauugacaauacgcuuutt

SEQ ID NO: 248 - DNA, 21 bp, Artificial Sequence
  Description of Artificial Sequence Synthetic oligonucleotide
aaagcguauugucaauaaatt

SEQ ID NO: 249 - DNA, 21 bp, Artificial Sequence
  Description of Artificial Sequence Synthetic oligonucleotide
cuuccucucgcaacaaacutt

SEQ ID NO: 250 - DNA, 21 bp, Artificial Sequence
  Description of Artificial Sequence Synthetic oligonucleotide
aguuuguugcgagaggaagtt

SEQ ID NO: 251 - DNA, 21 bp, Artificial Sequence
  Description of Artificial Sequence Synthetic oligonucleotide
gaccauauuuauuauaagutt

SEQ ID NO: 252 - DNA, 21 bp, Artificial Sequence
  Description of Artificial Sequence Synthetic oligonucleotide
acuuauaauaaauaugguctt

SEQ ID NO: 253 - DNA, 21 bp, Artificial Sequence
  Description of Artificial Sequence Synthetic oligonucleotide
ugaccaaggaaaucggccutt

SEQ ID NO: 254 - DNA, 21 bp, Artificial Sequence
  Description of Artificial Sequence Synthetic oligonucleotide
aggccgauuuccuuggucatt

SEQ ID NO: 255 - DNA, 21 bp, Artificial Sequence
  Description of Artificial Sequence Synthetic oligonucleotide
ugaccaaggaaaucggccutt

SEQ ID NO: 256 - DNA, 21 bp, Artificial Sequence
  Description of Artificial Sequence Synthetic oligonucleotide
aggccgauuuccuuggucatt

SEQ ID NO: 257 - DNA, 21 bp, Artificial Sequence
  Description of Artificial Sequence Synthetic oligonucleotide
ugaccaaggaaaucggccutt

SEQ ID NO: 258 - DNA, 21 bp, Artificial Sequence
  Description of Artificial Sequence Synthetic oligonucleotide
aggccgauuuccuuggucatt

SEQ ID NO: 259 - DNA, 21 bp, Artificial Sequence
  Description of Artificial Sequence Synthetic oligonucleotide
ugaccaaggaaaucggccutt

SEQ ID NO: 260 - DNA, 21 bp, Artificial Sequence
  Description of Artificial Sequence Synthetic oligonucleotide
aggccgauuuccuuggucatt

SEQ ID NO: 261 - DNA, 21 bp, Artificial Sequence
  Description of Artificial Sequence Synthetic oligonucleotide
tgaccaaggaaaucggccutt

SEQ ID NO: 262 - DNA, 21 bp, Artificial Sequence
  Description of Artificial Sequence Synthetic oligonucleotide
aggccgauuuccuuggucatt

SEQ ID NO: 263 - DNA, 21 bp, Artificial Sequence
  Description of Artificial Sequence Synthetic oligonucleotide
tgaccaaggaaaucggccutt

SEQ ID NO: 264 - DNA, 21 bp, Artificial Sequence
  Description of Artificial Sequence Synthetic oligonucleotide
aggccgauuuccuuggucatt

SEQ ID NO: 265 - DNA, 21 bp, Artificial Sequence
  Description of Artificial Sequence Synthetic oligonucleotide
tgaccaaggaaaucggccutt

SEQ ID NO: 266 - DNA, 21 bp, Artificial Sequence
  Description of Artificial Sequence Synthetic oligonucleotide
aggccgauuuccuuggucatt

SEQ ID NO: 267 - DNA, 21 bp, Artificial Sequence
  Description of Artificial Sequence Synthetic oligonucleotide
tgaccaaggaaaucggccutt

SEQ ID NO: 268 - DNA, 21 bp, Artificial Sequence
  Description of Artificial Sequence Synthetic oligonucleotide
aggccgauuuccuuggucatt

SEQ ID NO: 269 - DNA, 21 bp, Artificial Sequence
  Description of Artificial Sequence Synthetic oligonucleotide
ugaccaaggaaaucggccutt

SEQ ID NO: 270 - DNA, 21 bp, Artificial Sequence
  Description of Artificial Sequence Synthetic oligonucleotide
aggccgauuuccuuggucatt

SEQ ID NO: 271 - DNA, 21 bp, Artificial Sequence
  Description of Artificial Sequence Synthetic oligonucleotide
ugaccaaggaaaucggccutt

SEQ ID NO: 272 - DNA, 21 bp, Artificial Sequence
  Description of Artificial Sequence Synthetic oligonucleotide
aggccgauuuccuuggucatt

SEQ ID NO: 273 - DNA, 21 bp, Artificial Sequence
  Description of Artificial Sequence Synthetic oligonucleotide
ugaccaaggaaaucggccutt

SEQ ID NO: 274 - DNA, 21 bp, Artificial Sequence
  Description of Artificial Sequence Synthetic oligonucleotide
aggccgauuuccuuggucatt

SEQ ID NO: 275 - DNA, 21 bp, Artificial Sequence
  Description of Artificial Sequence Synthetic oligonucleotide
ugaccaaggaaaucggccutt

SEQ ID NO: 276 - DNA, 21 bp, Artificial Sequence
  Description of Artificial Sequence Synthetic oligonucleotide
aggccgauuuccuuggucatt

SEQ ID NO: 277 - DNA, 21 bp, Artificial Sequence
  Description of Artificial Sequence Synthetic oligonucleotide
ucaaaacgaagacuagcuatt

SEQ ID NO: 278 - DNA, 21 bp, Artificial Sequence
  Description of Artificial Sequence Synthetic oligonucleotide
uagcuagucuucguuuugatt

SEQ ID NO: 279 - DNA, 21 bp, Artificial Sequence
  Description of Artificial Sequence Synthetic oligonucleotide
gaaucuucaucauacgaagtt

SEQ ID NO: 280 - DNA, 21 bp, Artificial Sequence
  Description of Artificial Sequence Synthetic oligonucleotide
cuucguaugaugaagauuctt

SEQ ID NO: 281 - DNA, 21 bp, Artificial Sequence
  Description of Artificial Sequence Synthetic oligonucleotide
gaaucuucaucauacgaagtt

SEQ ID NO: 282 - DNA, 21 bp, Artificial Sequence
  Description of Artificial Sequence Synthetic oligonucleotide
cuucguaugaugaagauuctt

SEQ ID NO: 283 - DNA, 21 bp, Artificial Sequence
  Description of Artificial Sequence Synthetic oligonucleotide
gaaucuucaucauacgaagtt

SEQ ID NO: 284 - DNA, 21 bp, Artificial Sequence
  Description of Artificial Sequence Synthetic oligonucleotide
cuucguaugaugaagauuctt

SEQ ID NO: 285 - DNA, 21 bp, Artificial Sequence
  Description of Artificial Sequence Synthetic oligonucleotide
gaaucuucaucauacgaagtt

SEQ ID NO: 286 - DNA, 21 bp, Artificial Sequence
  Description of Artificial Sequence Synthetic oligonucleotide
cuucguaugaugaagauuctt

SEQ ID NO: 287 - DNA, 21 bp, Artificial Sequence
  Description of Artificial Sequence Synthetic oligonucleotide
taaucuucaucauacgaagtt

SEQ ID NO: 288 - DNA, 21 bp, Artificial Sequence
  Description of Artificial Sequence Synthetic oligonucleotide
cuucguaugaugaagauuctt

SEQ ID NO: 289 - DNA, 21 bp, Artificial Sequence
  Description of Artificial Sequence Synthetic oligonucleotide
taaucuucaucauacgaagtt

SEQ ID NO: 290 - DNA, 21 bp, Artificial Sequence
  Description of Artificial Sequence Synthetic oligonucleotide
cuucguaugaugaagauuctt

SEQ ID NO: 291 - DNA, 21 bp, Artificial Sequence
  Description of Artificial Sequence Synthetic oligonucleotide
taaucuucaucauacgaagtt

SEQ ID NO: 292 - DNA, 21 bp, Artificial Sequence
  Description of Artificial Sequence Synthetic oligonucleotide
cuucguaugaugaagauuctt

SEQ ID NO: 293 - DNA, 21 bp, Artificial Sequence
  Description of Artificial Sequence Synthetic oligonucleotide
taaucuucaucauacgaagtt

SEQ ID NO: 294 - DNA, 21 bp, Artificial Sequence
  Description of Artificial Sequence Synthetic oligonucleotide
cuucguaugaugaagauuctt

SEQ ID NO: 295 - DNA, 21 bp, Artificial Sequence
  Description of Artificial Sequence Synthetic oligonucleotide
gaaucuucaucauacgaagtt

SEQ ID NO: 296 - DNA, 21 bp, Artificial Sequence
  Description of Artificial Sequence Synthetic oligonucleotide
cuucguaugaugaagauuctt

SEQ ID NO: 297 - DNA, 21 bp, Artificial Sequence
  Description of Artificial Sequence Synthetic oligonucleotide
gaaucuucaucauacgaagtt

SEQ ID NO: 298 - DNA, 21 bp, Artificial Sequence
  Description of Artificial Sequence Synthetic oligonucleotide
cuucguaugaugaagauuctt

SEQ ID NO: 299 - DNA, 21 bp, Artificial Sequence
  Description of Artificial Sequence Synthetic oligonucleotide
gaaucuucaucauacgaagtt

SEQ ID NO: 300 - DNA, 21 bp, Artificial Sequence
  Description of Artificial Sequence Synthetic oligonucleotide
cuucguaugaugaagauuctt

SEQ ID NO: 301 - DNA, 21 bp, Artificial Sequence
  Description of Artificial Sequence Synthetic oligonucleotide
gaaucuucaucauacgaagtt

SEQ ID NO: 302 - DNA, 21 bp, Artificial Sequence
  Description of Artificial Sequence Synthetic oligonucleotide
cuucguaugaugaagauuctt

SEQ ID NO: 303 - DNA, 21 bp, Artificial Sequence
  Description of Artificial Sequence Synthetic oligonucleotide
gggauagaucuauaauguutt

SEQ ID NO: 304 - DNA, 21 bp, Artificial Sequence
  Description of Artificial Sequence Synthetic oligonucleotide
aacauuauagaucuauccctt

SEQ ID NO: 305 - DNA, 21 bp, Artificial Sequence
  Description of Artificial Sequence Synthetic oligonucleotide
cuauuugucgcaggaauaatt

SEQ ID NO: 306 - DNA, 21 bp, Artificial Sequence
  Description of Artificial Sequence Synthetic oligonucleotide
uuauuccugcgacaaauagtt

SEQ ID NO: 307 - DNA, 21 bp, Artificial Sequence
  Description of Artificial Sequence Synthetic oligonucleotide
ucuauuugaagauaugacutt

SEQ ID NO: 308 - DNA, 21 bp, Artificial Sequence
  Description of Artificial Sequence Synthetic oligonucleotide
agucauaucuucaaauagatt

SEQ ID NO: 309 - DNA, 21 bp, Artificial Sequence
  Description of Artificial Sequence Synthetic oligonucleotide
ucuauuugaagauaugacutt

SEQ ID NO: 310 - DNA, 21 bp, Artificial Sequence
  Description of Artificial Sequence Synthetic oligonucleotide
agucauaucuucaaauagatt

SEQ ID NO: 311 - DNA, 21 bp, Artificial Sequence
  Description of Artificial Sequence Synthetic oligonucleotide
ucuauuugaagauaugacutt

SEQ ID NO: 312 - DNA, 21 bp, Artificial Sequence
  Description of Artificial Sequence Synthetic oligonucleotide
agucauaucuucaaauagatt

SEQ ID NO: 313 - DNA, 21 bp, Artificial Sequence
  Description of Artificial Sequence Synthetic oligonucleotide
tcuauuugaagauaugacutt

SEQ ID NO: 314 - DNA, 21 bp, Artificial Sequence
  Description of Artificial Sequence Synthetic oligonucleotide
agucauaucuucaaauagatt

SEQ ID NO: 315 - DNA, 21 bp, Artificial Sequence
  Description of Artificial Sequence Synthetic oligonucleotide
tcuauuugaagauaugacutt

SEQ ID NO: 316 - DNA, 21 bp, Artificial Sequence
  Description of Artificial Sequence Synthetic oligonucleotide
agucauaucuucaaauagatt

SEQ ID NO: 317 - DNA, 21 bp, Artificial Sequence
  Description of Artificial Sequence Synthetic oligonucleotide
tcuauuugaagauaugacutt

SEQ ID NO: 318 - DNA, 21 bp, Artificial Sequence
  Description of Artificial Sequence Synthetic oligonucleotide
agucauaucuucaaauagatt

SEQ ID NO: 319 - DNA, 21 bp, Artificial Sequence
  Description of Artificial Sequence Synthetic oligonucleotide
ucuauuugaagauaugacutt

SEQ ID NO: 320 - DNA, 21 bp, Artificial Sequence
  Description of Artificial Sequence Synthetic oligonucleotide
agucauaucuucaaauagatt

SEQ ID NO: 321 - DNA, 21 bp, Artificial Sequence
  Description of Artificial Sequence Synthetic oligonucleotide
ucuauuugaagauaugacutt

SEQ ID NO: 322 - DNA, 21 bp, Artificial Sequence
  Description of Artificial Sequence Synthetic oligonucleotide
agucauaucuucaaauagatt

SEQ ID NO: 323 - DNA, 21 bp, Artificial Sequence
  Description of Artificial Sequence Synthetic oligonucleotide
ucuauuugaagauaugacutt

SEQ ID NO: 324 - DNA, 21 bp, Artificial Sequence
  Description of Artificial Sequence Synthetic oligonucleotide
agucauaucuucaaauagatt

SEQ ID NO: 325 - DNA, 21 bp, Artificial Sequence
  Description of Artificial Sequence Synthetic oligonucleotide
caacaaacuauuugucgcatt

SEQ ID NO: 326 - DNA, 21 bp, Artificial Sequence
  Description of Artificial Sequence Synthetic oligonucleotide
ugcgacaaauaguuuguugtt

SEQ ID NO: 327 - DNA, 21 bp, Artificial Sequence
  Description of Artificial Sequence Synthetic oligonucleotide
aucuucaucauacgaaggatt

SEQ ID NO: 328 - DNA, 21 bp, Artificial Sequence
  Description of Artificial Sequence Synthetic oligonucleotide
uccuucguaugaugaagautt

SEQ ID NO: 329 - DNA, 21 bp, Artificial Sequence
  Description of Artificial Sequence Synthetic oligonucleotide
cucucgcaacaaacuauuutt

SEQ ID NO: 330 - DNA, 21 bp, Artificial Sequence
  Description of Artificial Sequence Synthetic oligonucleotide
aaauaguuuguugcgagagtt

SEQ ID NO: 331 - DNA, 21 bp, Artificial Sequence
  Description of Artificial Sequence Synthetic oligonucleotide
ugaaucuucaucauacgaatt

SEQ ID NO: 332 - DNA, 21 bp, Artificial Sequence
  Description of Artificial Sequence Synthetic oligonucleotide
uucguaugaugaagauucatt

SEQ ID NO: 333 - DNA, 21 bp, Artificial Sequence
  Description of Artificial Sequence Synthetic oligonucleotide
cagaccuuccagaucgcuutt

SEQ ID NO: 334 - DNA, 21 bp, Artificial Sequence
  Description of Artificial Sequence Synthetic oligonucleotide
aagcgaucuggaaggucugtt

SEQ ID NO: 335 - DNA, 21 bp, Artificial Sequence
  Description of Artificial Sequence Synthetic oligonucleotide
cagaucgcuuccucucgcatt

SEQ ID NO: 336 - DNA, 21 bp, Artificial Sequence
  Description of Artificial Sequence Synthetic oligonucleotide
ugcgagaggaagcgaucugtt

SEQ ID NO: 337 - DNA, 21 bp, Artificial Sequence
  Description of Artificial Sequence Synthetic oligonucleotide
cuagagguauggcuguaactt

SEQ ID NO: 338 - DNA, 21 bp, Artificial Sequence
  Description of Artificial Sequence Synthetic oligonucleotide
guuacagccauaccucuagtt

SEQ ID NO: 339 - DNA, 21 bp, Artificial Sequence
  Description of Artificial Sequence Synthetic oligonucleotide
auuuauugacaauacgcuutt

SEQ ID NO: 340 - DNA, 21 bp, Artificial Sequence
  Description of Artificial Sequence Synthetic oligonucleotide
aagcguauugucaauaaautt

SEQ ID NO: 341 - DNA, 21 bp, Artificial Sequence
  Description of Artificial Sequence Synthetic oligonucleotide
acaauacgcuuuacuuuautt

SEQ ID NO: 342 - DNA, 21 bp, Artificial Sequence
  Description of Artificial Sequence Synthetic oligonucleotide
auaaaguaaagcguauugutt

SEQ ID NO: 343 - DNA, 21 bp, Artificial Sequence
  Description of Artificial Sequence Synthetic oligonucleotide
acgaaggauacuuucuagctt

SEQ ID NO: 344 - DNA, 21 bp, Artificial Sequence
  Description of Artificial Sequence Synthetic oligonucleotide
gcuagaaaguauccuucgutt

SEQ ID NO: 345 - DNA, 21 bp, Artificial Sequence
  Description of Artificial Sequence Synthetic oligonucleotide
ggaauugucucccagugcatt

SEQ ID NO: 346 - DNA, 21 bp, Artificial Sequence
  Description of Artificial Sequence Synthetic oligonucleotide
ugcacugggagacaauucctt

SEQ ID NO: 347 - DNA, 21 bp, Artificial Sequence
  Description of Artificial Sequence Synthetic oligonucleotide
cagucuacacagcuucgggtt

SEQ ID NO: 348 - DNA, 21 bp, Artificial Sequence
  Description of Artificial Sequence Synthetic oligonucleotide
cccgaagcuguguagacugtt

SEQ ID NO: 349 - DNA, 21 bp, Artificial Sequence
  Description of Artificial Sequence Synthetic oligonucleotide
aucauacgaaggauacuuutt

SEQ ID NO: 350 - DNA, 21 bp, Artificial Sequence
  Description of Artificial Sequence Synthetic oligonucleotide
aaaguauccuucguaugautt

SEQ ID NO: 351 - DNA, 21 bp, Artificial Sequence
  Description of Artificial Sequence Synthetic oligonucleotide
uugaaucuucaucauacgatt

SEQ ID NO: 352 - DNA, 21 bp, Artificial Sequence
  Description of Artificial Sequence Synthetic oligonucleotide
ucguaugaugaagauucaatt

SEQ ID NO: 353 - DNA, 21 bp, Artificial Sequence
  Description of Artificial Sequence Synthetic oligonucleotide
uuugaaucuucaucauacgtt

SEQ ID NO: 354 - DNA, 21 bp, Artificial Sequence
  Description of Artificial Sequence Synthetic oligonucleotide
cguaugaugaagauucaaatt

SEQ ID NO: 355 - DNA, 21 bp, Artificial Sequence
  Description of Artificial Sequence Synthetic oligonucleotide
gaucgcuuccucucgcaactt

SEQ ID NO: 356 - DNA, 21 bp, Artificial Sequence
  Description of Artificial Sequence Synthetic oligonucleotide
guugcgagaggaagcgauctt

SEQ ID NO: 357 - DNA, 21 bp, Artificial Sequence
  Description of Artificial Sequence Synthetic oligonucleotide
gagguauggcuguaacuautt

SEQ ID NO: 358 - DNA, 21 bp, Artificial Sequence
  Description of Artificial Sequence Synthetic oligonucleotide
auaguuacagccauaccuctt

SEQ ID NO: 359 - DNA, 21 bp, Artificial Sequence
  Description of Artificial Sequence Synthetic oligonucleotide
ucccaggacaugauaauaatt

SEQ ID NO: 360 - DNA, 21 bp, Artificial Sequence
  Description of Artificial Sequence Synthetic oligonucleotide
uuauuaucauguccugggatt

SEQ ID NO: 361 - DNA, 21 bp, Artificial Sequence
  Description of Artificial Sequence Synthetic oligonucleotide
ucuacacagcuucgggaagtt

SEQ ID NO: 362 - DNA, 21 bp, Artificial Sequence
  Description of Artificial Sequence Synthetic oligonucleotide
cuucccgaagcuguguagatt

SEQ ID NO: 363 - DNA, 21 bp, Artificial Sequence
  Description of Artificial Sequence Synthetic oligonucleotide
ucucgcaacaaacuauuugtt

SEQ ID NO: 364 - DNA, 21 bp, Artificial Sequence
  Description of Artificial Sequence Synthetic oligonucleotide
caaauaguuuguugcgagatt

SEQ ID NO: 365 - DNA, 21 bp, Artificial Sequence
  Description of Artificial Sequence Synthetic oligonucleotide
gaaucacuugcacuccggatt

SEQ ID NO: 366 - DNA, 21 bp, Artificial Sequence
  Description of Artificial Sequence Synthetic oligonucleotide
uccggagugcaagugauuctt

SEQ ID NO: 367 - DNA, 21 bp, Artificial Sequence
  Description of Artificial Sequence Synthetic oligonucleotide
ugaaucuaaauuaucaguctt

SEQ ID NO: 368 - DNA, 21 bp, Artificial Sequence
  Description of Artificial Sequence Synthetic oligonucleotide
gacugauaauuuagauucatt

SEQ ID NO: 369 - DNA, 21 bp, Artificial Sequence
  Description of Artificial Sequence Synthetic oligonucleotide
gaauccuccugauaacauctt

SEQ ID NO: 370 - DNA, 21 bp, Artificial Sequence
  Description of Artificial Sequence Synthetic oligonucleotide
gauguuaucaggaggauuctt

SEQ ID NO: 371 - DNA, 21 bp, Artificial Sequence
  Description of Artificial Sequence Synthetic oligonucleotide
agagguauggcuguaacuatt

SEQ ID NO: 372 - DNA, 21 bp, Artificial Sequence
  Description of Artificial Sequence Synthetic oligonucleotide
uaguuacagccauaccucutt

SEQ ID NO: 373 - DNA, 21 bp, Artificial Sequence
  Description of Artificial Sequence Synthetic oligonucleotide
gucccaggacaugauaauatt

SEQ ID NO: 374 - DNA, 21 bp, Artificial Sequence
  Description of Artificial Sequence Synthetic oligonucleotide
uauuaucauguccugggactt

SEQ ID NO: 375 - DNA, 21 bp, Artificial Sequence
  Description of Artificial Sequence Synthetic oligonucleotide
gauaacaucaaggauacaatt

SEQ ID NO: 376 - DNA, 21 bp, Artificial Sequence
  Description of Artificial Sequence Synthetic oligonucleotide
uuguauccuugauguuauctt

SEQ ID NO: 377 - DNA, 21 bp, Artificial Sequence
  Description of Artificial Sequence Synthetic oligonucleotide
acaaacuauuugucgcaggtt

SEQ ID NO: 378 - DNA, 21 bp, Artificial Sequence
  Description of Artificial Sequence Synthetic oligonucleotide
ccugcgacaaauaguuugutt

SEQ ID NO: 379 - DNA, 21 bp, Artificial Sequence
  Description of Artificial Sequence Synthetic oligonucleotide
aaggauacuuucuagcuugtt

SEQ ID NO: 380 - DNA, 21 bp, Artificial Sequence
  Description of Artificial Sequence Synthetic oligonucleotide
caagcuagaaaguauccuutt

SEQ ID NO: 381 - DNA, 21 bp, Artificial Sequence
  Description of Artificial Sequence Synthetic oligonucleotide
uauuaaaauuucaugccggtt

SEQ ID NO: 382 - DNA, 21 bp, Artificial Sequence
  Description of Artificial Sequence Synthetic oligonucleotide
ccggcaugaaauuuuaauatt

SEQ ID NO: 383 - DNA, 21 bp, Artificial Sequence
  Description of Artificial Sequence Synthetic oligonucleotide
uagccagccuagagguaugtt

SEQ ID NO: 384 - DNA, 21 bp, Artificial Sequence
  Description of Artificial Sequence Synthetic oligonucleotide
cauaccucuaggcuggcuatt

SEQ ID NO: 385 - DNA, 21 bp, Artificial Sequence
  Description of Artificial Sequence Synthetic oligonucleotide
uggcugcugaaccaguagatt

SEQ ID NO: 386 - DNA, 21 bp, Artificial Sequence
  Description of Artificial Sequence Synthetic oligonucleotide
ucuacugguucagcagccatt

SEQ ID NO: 387 - DNA, 21 bp, Artificial Sequence
  Description of Artificial Sequence Synthetic oligonucleotide
agaaucacuugcacuccggtt

SEQ ID NO: 388 - DNA, 21 bp, Artificial Sequence
  Description of Artificial Sequence Synthetic oligonucleotide
ccggagugcaagugauucutt

SEQ ID NO: 389 - DNA, 21 bp, Artificial Sequence
  Description of Artificial Sequence Synthetic oligonucleotide
auuaaaauuucaugccgggtt

SEQ ID NO: 390 - DNA, 21 bp, Artificial Sequence
  Description of Artificial Sequence Synthetic oligonucleotide
cccggcaugaaauuuuaautt

SEQ ID NO: 391 - DNA, 21 bp, Artificial Sequence
  Description of Artificial Sequence Synthetic oligonucleotide
ugcagucuacacagcuucgtt

SEQ ID NO: 392 - DNA, 21 bp, Artificial Sequence
  Description of Artificial Sequence Synthetic oligonucleotide
cgaagcuguguagacugcatt

SEQ ID NO: 393 - DNA, 21 bp, Artificial Sequence
  Description of Artificial Sequence Synthetic oligonucleotide
gaucuauaauguucacugutt

SEQ ID NO: 394 - DNA, 21 bp, Artificial Sequence
  Description of Artificial Sequence Synthetic oligonucleotide
acagugaacauuauagauctt

SEQ ID NO: 395 - DNA, 21 bp, Artificial Sequence
  Description of Artificial Sequence Synthetic oligonucleotide
gcagucuacacagcuucggtt

SEQ ID NO: 396 - DNA, 21 bp, Artificial Sequence
  Description of Artificial Sequence Synthetic oligonucleotide
ccgaagcuguguagacugctt

SEQ ID NO: 397 - DNA, 21 bp, Artificial Sequence
  Description of Artificial Sequence Synthetic oligonucleotide
uuaaaauuucaugccgggctt

SEQ ID NO: 398 - DNA, 21 bp, Artificial Sequence
  Description of Artificial Sequence Synthetic oligonucleotide
gcccggcaugaaauuuuaatt

SEQ ID NO: 399 - DNA, 21 bp, Artificial Sequence
  Description of Artificial Sequence Synthetic oligonucleotide
aacaaacuauuugucgcagtt

SEQ ID NO: 400 - DNA, 21 bp, Artificial Sequence
  Description of Artificial Sequence Synthetic oligonucleotide
cugcgacaaauaguuuguutt

SEQ ID NO: 401 - DNA, 21 bp, Artificial Sequence
  Description of Artificial Sequence Synthetic oligonucleotide
aauuuauugacaauacgcutt

SEQ ID NO: 402 - DNA, 21 bp, Artificial Sequence
  Description of Artificial Sequence Synthetic oligonucleotide
agcguauugucaauaaauutt

SEQ ID NO: 403 - DNA, 21 bp, Artificial Sequence
  Description of Artificial Sequence Synthetic oligonucleotide
aaaauuucaugccgggcgctt

SEQ ID NO: 404 - DNA, 21 bp, Artificial Sequence
  Description of Artificial Sequence Synthetic oligonucleotide
gcgcccggcaugaaauuuutt

SEQ ID NO: 405 - DNA, 21 bp, Artificial Sequence
  Description of Artificial Sequence Synthetic oligonucleotide
guguagugacgcaugcccutt

SEQ ID NO: 406 - DNA, 21 bp, Artificial Sequence
  Description of Artificial Sequence Synthetic oligonucleotide
agggcaugcgucacuacactt

SEQ ID NO: 407 - DNA, 21 bp, Artificial Sequence
  Description of Artificial Sequence Synthetic oligonucleotide
aaauuuauugacaauacgctt

SEQ ID NO: 408 - DNA, 21 bp, Artificial Sequence
  Description of Artificial Sequence Synthetic oligonucleotide
gcguauugucaauaaauuutt

SEQ ID NO: 409 - DNA, 21 bp, Artificial Sequence
  Description of Artificial Sequence Synthetic oligonucleotide
auugaccaaggaaaucggctt

SEQ ID NO: 410 - DNA, 21 bp, Artificial Sequence
  Description of Artificial Sequence Synthetic oligonucleotide
gccgauuuccuuggucaautt

SEQ ID NO: 411 - DNA, 21 bp, Artificial Sequence
  Description of Artificial Sequence Synthetic oligonucleotide
uguagugacgcaugcccuctt

SEQ ID NO: 412 - DNA, 21 bp, Artificial Sequence
  Description of Artificial Sequence Synthetic oligonucleotide
gagggcaugcgucacuacatt

SEQ ID NO: 413 - DNA, 21 bp, Artificial Sequence
  Description of Artificial Sequence Synthetic oligonucleotide
ccggaccauauuuauuauatt

SEQ ID NO: 414 - DNA, 21 bp, Artificial Sequence
  Description of Artificial Sequence Synthetic oligonucleotide
uauaauaaauaugguccggtt

SEQ ID NO: 415 - DNA, 21 bp, Artificial Sequence
  Description of Artificial Sequence Synthetic oligonucleotide
agccagccuagagguauggtt

SEQ ID NO: 416 - DNA, 21 bp, Artificial Sequence
  Description of Artificial Sequence Synthetic oligonucleotide
ccauaccucuaggcuggcutt

SEQ ID NO: 417 - DNA, 21 bp, Artificial Sequence
  Description of Artificial Sequence Synthetic oligonucleotide
guagugacgcaugcccucatt

SEQ ID NO: 418 - DNA, 21 bp, Artificial Sequence
  Description of Artificial Sequence Synthetic oligonucleotide
ugagggcaugcgucacuactt

SEQ ID NO: 419 - DNA, 21 bp, Artificial Sequence
  Description of Artificial Sequence Synthetic oligonucleotide
gacgcaugcccucaauccctt

SEQ ID NO: 420 - DNA, 21 bp, Artificial Sequence
  Description of Artificial Sequence Synthetic oligonucleotide
gggauugagggcaugcguctt

SEQ ID NO: 421 - DNA, 21 bp, Artificial Sequence
  Description of Artificial Sequence Synthetic oligonucleotide
aaucuucaucauacgaaggtt

SEQ ID NO: 422 - DNA, 21 bp, Artificial Sequence
  Description of Artificial Sequence Synthetic oligonucleotide
ccuucguaugaugaagauutt

SEQ ID NO: 423 - DNA, 21 bp, Artificial Sequence
  Description of Artificial Sequence Synthetic oligonucleotide
ggaaaucggccucuauuugtt

SEQ ID NO: 424 - DNA, 21 bp, Artificial Sequence
  Description of Artificial Sequence Synthetic oligonucleotide
caaauagaggccgauuucctt

SEQ ID NO: 425 - DNA, 21 bp, Artificial Sequence
  Description of Artificial Sequence Synthetic oligonucleotide
ucagaccuuccagaucgcutt

SEQ ID NO: 426 - DNA, 21 bp, Artificial Sequence
  Description of Artificial Sequence Synthetic oligonucleotide
agcgaucuggaaggucugatt

SEQ ID NO: 427 - DNA, 21 bp, Artificial Sequence
  Description of Artificial Sequence Synthetic oligonucleotide
auaugacugauucugacugtt

SEQ ID NO: 428 - DNA, 21 bp, Artificial Sequence
  Description of Artificial Sequence Synthetic oligonucleotide
cagucagaaucagucauautt

SEQ ID NO: 429 - DNA, 21 bp, Artificial Sequence
  Description of Artificial Sequence Synthetic oligonucleotide
uugcacuccggagguagagtt

SEQ ID NO: 430 - DNA, 21 bp, Artificial Sequence
  Description of Artificial Sequence Synthetic oligonucleotide
cucuaccuccggagugcaatt

SEQ ID NO: 431 - DNA, 21 bp, Artificial Sequence
  Description of Artificial Sequence Synthetic oligonucleotide
cuugcacuccggagguagatt

SEQ ID NO: 432 - DNA, 21 bp, Artificial Sequence
  Description of Artificial Sequence Synthetic oligonucleotide
ucuaccuccggagugcaagtt

SEQ ID NO: 433 - DNA, 21 bp, Artificial Sequence
  Description of Artificial Sequence Synthetic oligonucleotide
ugacgcaugcccucaaucctt

SEQ ID NO: 434 - DNA, 21 bp, Artificial Sequence
  Description of Artificial Sequence Synthetic oligonucleotide
ggauugagggcaugcgucatt

SEQ ID NO: 435 - DNA, 21 bp, Artificial Sequence
  Description of Artificial Sequence Synthetic oligonucleotide
ucauacgaaggauacuuuctt

SEQ ID NO: 436 - DNA, 21 bp, Artificial Sequence
  Description of Artificial Sequence Synthetic oligonucleotide
gaaaguauccuucguaugatt

SEQ ID NO: 437 - DNA, 21 bp, Artificial Sequence
  Description of Artificial Sequence Synthetic oligonucleotide
ucauacgaaggauacuuuctt

SEQ ID NO: 438 - DNA, 21 bp, Artificial Sequence
  Description of Artificial Sequence Synthetic oligonucleotide
gaaaguauccuucguaugatt

SEQ ID NO: 439 - DNA, 21 bp, Artificial Sequence
  Description of Artificial Sequence Synthetic oligonucleotide
ucauacgaaggauacuuuctt

SEQ ID NO: 440 - DNA, 21 bp, Artificial Sequence
  Description of Artificial Sequence Synthetic oligonucleotide
gaaaguauccuucguaugatt

SEQ ID NO: 441 - DNA, 21 bp, Artificial Sequence
  Description of Artificial Sequence Synthetic oligonucleotide
cauugaccaaggaaaucggtt

SEQ ID NO: 442 - DNA, 21 bp, Artificial Sequence
  Description of Artificial Sequence Synthetic oligonucleotide
ccgauuuccuuggucaaugtt

SEQ ID NO: 443 - DNA, 21 bp, Artificial Sequence
  Description of Artificial Sequence Synthetic oligonucleotide
cauugaccaaggaaaucggtt

SEQ ID NO: 444 - DNA, 21 bp, Artificial Sequence
  Description of Artificial Sequence Synthetic oligonucleotide
ccgauuuccuuggucaaugtt

SEQ ID NO: 445 - DNA, 21 bp, Artificial Sequence
  Description of Artificial Sequence Synthetic oligonucleotide
cauugaccaaggaaaucggtt

SEQ ID NO: 446 - DNA, 21 bp, Artificial Sequence
  Description of Artificial Sequence Synthetic oligonucleotide
ccgauuuccuuggucaaugtt

SEQ ID NO: 447 - DNA, 21 bp, Artificial Sequence
  Description of Artificial Sequence Synthetic oligonucleotide
cauugaccaaggaaaucggtt

SEQ ID NO: 448 - DNA, 21 bp, Artificial Sequence
  Description of Artificial Sequence Synthetic oligonucleotide
ccgauuuccuuggucaaugtt

SEQ ID NO: 449 - DNA, 21 bp, Artificial Sequence
  Description of Artificial Sequence Synthetic oligonucleotide
cauugaccaaggaaaucggtt

SEQ ID NO: 450 - DNA, 21 bp, Artificial Sequence
  Description of Artificial Sequence Synthetic oligonucleotide
ccgauuuccuuggucaaugtt

SEQ ID NO: 451 - DNA, 21 bp, Artificial Sequence
  Description of Artificial Sequence Synthetic oligonucleotide
cccaggacaugauaauaagtt

SEQ ID NO: 452 - DNA, 21 bp, Artificial Sequence
  Description of Artificial Sequence Synthetic oligonucleotide
cuuauuaucauguccugggtt

SEQ ID NO: 453 - DNA, 21 bp, Artificial Sequence
  Description of Artificial Sequence Synthetic oligonucleotide
cccaggacaugauaauaagtt

SEQ ID NO: 454 - DNA, 21 bp, Artificial Sequence
  Description of Artificial Sequence Synthetic oligonucleotide
cuuauuaucauguccugggtt

SEQ ID NO: 455 - DNA, 21 bp, Artificial Sequence
  Description of Artificial Sequence Synthetic oligonucleotide
cccaggacaugauaauaagtt

SEQ ID NO: 456 - DNA, 21 bp, Artificial Sequence
  Description of Artificial Sequence Synthetic oligonucleotide
cuuauuaucauguccugggtt

SEQ ID NO: 457 - DNA, 21 bp, Artificial Sequence
  Description of Artificial Sequence Synthetic oligonucleotide
cccaggacaugauaauaagtt

SEQ ID NO: 458 - DNA, 21 bp, Artificial Sequence
  Description of Artificial Sequence Synthetic oligonucleotide
cuuauuaucauguccugggtt

SEQ ID NO: 459 - DNA, 21 bp, Artificial Sequence
  Description of Artificial Sequence Synthetic oligonucleotide
cccaggacaugauaauaagtt

SEQ ID NO: 460 - DNA, 21 bp, Artificial Sequence
  Description of Artificial Sequence Synthetic oligonucleotide
cuuauuaucauguccugggtt

SEQ ID NO: 461 - DNA, 21 bp, Artificial Sequence
  Description of Artificial Sequence Synthetic oligonucleotide
cccaggacaugauaauaagtt

SEQ ID NO: 462 - DNA, 21 bp, Artificial Sequence
  Description of Artificial Sequence Synthetic oligonucleotide
cuuauuaucauguccugggtt

SEQ ID NO: 463 - DNA, 21 bp, Artificial Sequence
  Description of Artificial Sequence Synthetic oligonucleotide
cccaggacaugauaauaagtt

SEQ ID NO: 464 - DNA, 21 bp, Artificial Sequence
  Description of Artificial Sequence Synthetic oligonucleotide
cuuauuaucauguccugggtt

SEQ ID NO: 465 - DNA, 21 bp, Artificial Sequence
  Description of Artificial Sequence Synthetic oligonucleotide
caaacuauuugucgcaggatt

SEQ ID NO: 466 - DNA, 21 bp, Artificial Sequence
  Description of Artificial Sequence Synthetic oligonucleotide
uccugcgacaaauaguuugtt

SEQ ID NO: 467 - DNA, 21 bp, Artificial Sequence
  Description of Artificial Sequence Synthetic oligonucleotide
caaacuauuugucgcaggatt

SEQ ID NO: 468 - DNA, 21 bp, Artificial Sequence
  Description of Artificial Sequence Synthetic oligonucleotide
uccugcgacaaauaguuugtt

SEQ ID NO: 469 - DNA, 21 bp, Artificial Sequence
  Description of Artificial Sequence Synthetic oligonucleotide
caaacuauuugucgcaggatt

SEQ ID NO: 470 - DNA, 21 bp, Artificial Sequence
  Description of Artificial Sequence Synthetic oligonucleotide
uccugcgacaaauaguuugtt

SEQ ID NO: 471 - DNA, 21 bp, Artificial Sequence
  Description of Artificial Sequence Synthetic oligonucleotide
caaacuauuugucgcaggatt

SEQ ID NO: 472 - DNA, 21 bp, Artificial Sequence
  Description of Artificial Sequence Synthetic oligonucleotide
uccugcgacaaauaguuugtt

SEQ ID NO: 473 - DNA, 21 bp, Artificial Sequence
  Description of Artificial Sequence Synthetic oligonucleotide
caaacuauuugucgcaggatt

SEQ ID NO: 474 - DNA, 21 bp, Artificial Sequence
  Description of Artificial Sequence Synthetic oligonucleotide
uccugcgacaaauaguuugtt

SEQ ID NO: 475 - DNA, 21 bp, Artificial Sequence
  Description of Artificial Sequence Synthetic oligonucleotide
caaacuauuugucgcaggatt

SEQ ID NO: 476 - DNA, 21 bp, Artificial Sequence
  Description of Artificial Sequence Synthetic oligonucleotide
uccugcgacaaauaguuugtt

SEQ ID NO: 477 - DNA, 21 bp, Artificial Sequence
  Description of Artificial Sequence Synthetic oligonucleotide
caaacuauuugucgcaggatt

SEQ ID NO: 478 - DNA, 21 bp, Artificial Sequence
  Description of Artificial Sequence Synthetic oligonucleotide
uccugcgacaaauaguuugtt

SEQ ID NO: 479 - DNA, 21 bp, Artificial Sequence
  Description of Artificial Sequence Synthetic oligonucleotide
caaacuauuugucgcaggatt

SEQ ID NO: 480 - DNA, 21 bp, Artificial Sequence
  Description of Artificial Sequence Synthetic oligonucleotide
uccugcgacaaauaguuugtt

SEQ ID NO: 481 - DNA, 21 bp, Artificial Sequence
  Description of Artificial Sequence Synthetic oligonucleotide
guauguauaaagauagccatt

SEQ ID NO: 482 - DNA, 21 bp, Artificial Sequence
  Description of Artificial Sequence Synthetic oligonucleotide
uggcuaucuuuauacauactt

SEQ ID NO: 483 - DNA, 21 bp, Artificial Sequence
  Description of Artificial Sequence Synthetic oligonucleotide
guauguauaaagauagccatt

SEQ ID NO: 484 - DNA, 21 bp, Artificial Sequence
  Description of Artificial Sequence Synthetic oligonucleotide
uggcuaucuuuauacauactt

SEQ ID NO: 485 - DNA, 21 bp, Artificial Sequence
  Description of Artificial Sequence Synthetic oligonucleotide
guauguauaaagauagccatt

SEQ ID NO: 486 - DNA, 21 bp, Artificial Sequence
  Description of Artificial Sequence Synthetic oligonucleotide
uggcuaucuuuauacauactt

SEQ ID NO: 487 - DNA, 21 bp, Artificial Sequence
  Description of Artificial Sequence Synthetic oligonucleotide
guauguauaaagauagccatt

SEQ ID NO: 488 - DNA, 21 bp, Artificial Sequence
  Description of Artificial Sequence Synthetic oligonucleotide
uggcuaucuuuauacauactt

SEQ ID NO: 489 - DNA, 21 bp, Artificial Sequence
  Description of Artificial Sequence Synthetic oligonucleotide
guauguauaaagauagccatt

SEQ ID NO: 490 - DNA, 21 bp, Artificial Sequence
  Description of Artificial Sequence Synthetic oligonucleotide
uggcuaucuuuauacauactt

SEQ ID NO: 491 - DNA, 21 bp, Artificial Sequence
  Description of Artificial Sequence Synthetic oligonucleotide
guauguauaaagauagccatt

SEQ ID NO: 492 - DNA, 21 bp, Artificial Sequence
  Description of Artificial Sequence Synthetic oligonucleotide
uggcuaucuuuauacauactt

SEQ ID NO: 493 - DNA, 21 bp, Artificial Sequence
  Description of Artificial Sequence Synthetic oligonucleotide
guauguauaaagauagccatt

SEQ ID NO: 494 - DNA, 21 bp, Artificial Sequence
  Description of Artificial Sequence Synthetic oligonucleotide
uggcuaucuuuauacauactt

SEQ ID NO: 495 - DNA, 21 bp, Artificial Sequence
  Description of Artificial Sequence Synthetic oligonucleotide
guauguauaaagauagccatt

SEQ ID NO: 496 - DNA, 21 bp, Artificial Sequence
  Description of Artificial Sequence Synthetic oligonucleotide
uggcuaucuuuauacauactt

SEQ ID NO: 497 - DNA, 21 bp, Artificial Sequence
  Description of Artificial Sequence Synthetic oligonucleotide
guauguauaaagauagccatt

SEQ ID NO: 498 - DNA, 21 bp, Artificial Sequence
  Description of Artificial Sequence Synthetic oligonucleotide
uggcuaucuuuauacauactt

SEQ ID NO: 499 - DNA, 21 bp, Artificial Sequence
  Description of Artificial Sequence Synthetic oligonucleotide
guauguauaaagauagccatt

SEQ ID NO: 500 - DNA, 21 bp, Artificial Sequence
  Description of Artificial Sequence Synthetic oligonucleotide
uggcuaucuuuauacauactt

SEQ ID NO: 501 - DNA, 21 bp, Artificial Sequence
  Description of Artificial Sequence Synthetic oligonucleotide
guauguauaaagauagccatt

SEQ ID NO: 502 - DNA, 21 bp, Artificial Sequence
  Description of Artificial Sequence Synthetic oligonucleotide
uggcuaucuuuauacauactt

SEQ ID NO: 503 - DNA, 21 bp, Artificial Sequence
  Description of Artificial Sequence Synthetic oligonucleotide
acucucuccugugagaacatt

SEQ ID NO: 504 - DNA, 21 bp, Artificial Sequence
  Description of Artificial Sequence Synthetic oligonucleotide
uguucucacaggagagagutt

SEQ ID NO: 505 - DNA, 21 bp, Artificial Sequence
  Description of Artificial Sequence Synthetic oligonucleotide
acucucuccugugagaacatt

SEQ ID NO: 506 - DNA, 21 bp, Artificial Sequence
  Description of Artificial Sequence Synthetic oligonucleotide
uguucucacaggagagagutt

SEQ ID NO: 507 - DNA, 21 bp, Artificial Sequence
  Description of Artificial Sequence Synthetic oligonucleotide
acucucuccugugagaacatt

SEQ ID NO: 508 - DNA, 21 bp, Artificial Sequence
  Description of Artificial Sequence Synthetic oligonucleotide
uguucucacaggagagagutt

SEQ ID NO: 509 - DNA, 21 bp, Artificial Sequence
  Description of Artificial Sequence Synthetic oligonucleotide
acucucuccugugagaacatt

SEQ ID NO: 510 - DNA, 21 bp, Artificial Sequence
  Description of Artificial Sequence Synthetic oligonucleotide
uguucucacaggagagagutt

SEQ ID NO: 511 - DNA, 21 bp, Artificial Sequence
  Description of Artificial Sequence Synthetic oligonucleotide
acucucuccugugagaacatt

SEQ ID NO: 512 - DNA, 21 bp, Artificial Sequence
  Description of Artificial Sequence Synthetic oligonucleotide
uguucucacaggagagagutt

SEQ ID NO: 513 - DNA, 21 bp, Artificial Sequence
  Description of Artificial Sequence Synthetic oligonucleotide
acucucuccugugagaacatt

SEQ ID NO: 514 - DNA, 21 bp, Artificial Sequence
  Description of Artificial Sequence Synthetic oligonucleotide
uguucucacaggagagagutt

SEQ ID NO: 515 - DNA, 21 bp, Artificial Sequence
  Description of Artificial Sequence Synthetic oligonucleotide
acucucuccugugagaacatt

SEQ ID NO: 516 - DNA, 21 bp, Artificial Sequence
  Description of Artificial Sequence Synthetic oligonucleotide
uguucucacaggagagagutt

SEQ ID NO: 517 - DNA, 21 bp, Artificial Sequence
  Description of Artificial Sequence Synthetic oligonucleotide
acucucuccugugagaacatt

SEQ ID NO: 518 - DNA, 21 bp, Artificial Sequence
  Description of Artificial Sequence Synthetic oligonucleotide
uguucucacaggagagagutt

SEQ ID NO: 519 - RNA, 19 nt, Artificial Sequence
  Description of Artificial Sequence Synthetic oligonucleotide
uguucgaggauaugacuga

SEQ ID NO: 520 - RNA, 19 nt, Artificial Sequence
  Description of Artificial Sequence Synthetic oligonucleotide
ucagucauauccucgaaca

SEQ ID NO: 521 - RNA, 19 nt, Artificial Sequence
  Description of Artificial Sequence Synthetic oligonucleotide
uucacuguacaaccgcagu

SEQ ID NO: 522 - RNA, 19 nt, Artificial Sequence
  Description of Artificial Sequence Synthetic oligonucleotide
acugcgguuguacagugaa

SEQ ID NO: 523 - RNA, 19 nt, Artificial Sequence
  Description of Artificial Sequence Synthetic oligonucleotide
ucucuucguugacaaaaga

SEQ ID NO: 524 - RNA, 19 nt, Artificial Sequence
  Description of Artificial Sequence Synthetic oligonucleotide
ucuuuugucaacgaagaga

SEQ ID NO: 525 - RNA, 19 nt, Artificial Sequence
  Description of Artificial Sequence Synthetic oligonucleotide
uaguaaaaugucuacccuc

SEQ ID NO: 526 - RNA, 19 nt, Artificial Sequence
  Description of Artificial Sequence Synthetic oligonucleotide
gaggguagacauuuuacua

SEQ ID NO: 527 - RNA, 19 nt, Artificial Sequence
  Description of Artificial Sequence Synthetic oligonucleotide
ucagaagacucuugcguca

SEQ ID NO: 528 - RNA, 19 nt, Artificial Sequence
  Description of Artificial Sequence Synthetic oligonucleotide
ugacgcaagagucuucuga

SEQ ID NO: 529 - RNA, 19 nt, Artificial Sequence
  Description of Artificial Sequence Synthetic oligonucleotide
cgcuuuacuuuauaccuga

SEQ ID NO: 530 - RNA, 19 nt, Artificial Sequence
  Description of Artificial Sequence Synthetic oligonucleotide
ucagguauaaaguaaagcg

SEQ ID NO: 531 - RNA, 19 nt, Artificial Sequence
  Description of Artificial Sequence Synthetic oligonucleotide
accagacugauaauauaca

SEQ ID NO: 532 - RNA, 19 nt, Artificial Sequence
  Description of Artificial Sequence Synthetic oligonucleotide
uguauauuaucagucuggu

SEQ ID NO: 533 - RNA, 19 nt, Artificial Sequence
  Description of Artificial Sequence Synthetic oligonucleotide
aaagacagccuguguucga

SEQ ID NO: 534 - RNA, 19 nt, Artificial Sequence
  Description of Artificial Sequence Synthetic oligonucleotide
ucgaacacaggcugucuuu

SEQ ID NO: 535 - RNA, 19 nt, Artificial Sequence
  Description of Artificial Sequence Synthetic oligonucleotide
cugugaaggauaguaaaau

SEQ ID NO: 536 - RNA, 19 nt, Artificial Sequence
  Description of Artificial Sequence Synthetic oligonucleotide
auuuuacuauccuucacag

SEQ ID NO: 537 - RNA, 19 nt, Artificial Sequence
  Description of Artificial Sequence Synthetic oligonucleotide
uuuauugacaacacgcuuu

SEQ ID NO: 538 - RNA, 19 nt, Artificial Sequence
  Description of Artificial Sequence Synthetic oligonucleotide
aaagcguguugucaauaaa

SEQ ID NO: 539 - RNA, 19 nt, Artificial Sequence
  Description of Artificial Sequence Synthetic oligonucleotide
ccauaacagaauacccgag

SEQ ID NO: 540 - RNA, 19 nt, Artificial Sequence
  Description of Artificial Sequence Synthetic oligonucleotide
cucggguauucuguuaugg

SEQ ID NO: 541 - RNA, 19 nt, Artificial Sequence
  Description of Artificial Sequence Synthetic oligonucleotide
ucaaggaaaugauguuuau

SEQ ID NO: 542 - RNA, 19 nt, Artificial Sequence
  Description of Artificial Sequence Synthetic oligonucleotide
auaaacaucauuuccuuga

SEQ ID NO: 543 - RNA, 19 nt, Artificial Sequence
  Description of Artificial Sequence Synthetic oligonucleotide
acuucacuguacaaccgca

SEQ ID NO: 544 - RNA, 19 nt, Artificial Sequence
  Description of Artificial Sequence Synthetic oligonucleotide
ugcgguuguacagugaagu

SEQ ID NO: 545 - RNA, 19 nt, Artificial Sequence
  Description of Artificial Sequence Synthetic oligonucleotide
accgcaguaauacggaaua

SEQ ID NO: 546 - RNA, 19 nt, Artificial Sequence
  Description of Artificial Sequence Synthetic oligonucleotide
uauuccguauuacugcggu

SEQ ID NO: 547 - RNA, 19 nt, Artificial Sequence
  Description of Artificial Sequence Synthetic oligonucleotide
aaagugaucucauauucuu

SEQ ID NO: 548 - RNA, 19 nt, Artificial Sequence
  Description of Artificial Sequence Synthetic oligonucleotide
aagaauaugagaucacuuu

SEQ ID NO: 549 - RNA, 19 nt, Artificial Sequence
  Description of Artificial Sequence Synthetic oligonucleotide
aggaagaugaugcuuucaa

SEQ ID NO: 550 - RNA, 19 nt, Artificial Sequence
  Description of Artificial Sequence Synthetic oligonucleotide
uugaaagcaucaucuuccu

SEQ ID NO: 551 - RNA, 19 nt, Artificial Sequence
  Description of Artificial Sequence Synthetic oligonucleotide
caaagaaagccgccucaaa

SEQ ID NO: 552 - RNA, 19 nt, Artificial Sequence
  Description of Artificial Sequence Synthetic oligonucleotide
uuugaggcggcuuucuuug

SEQ ID NO: 553 - RNA, 19 nt, Artificial Sequence
  Description of Artificial Sequence Synthetic oligonucleotide
ugauguuuauugacaacac

SEQ ID NO: 554 - RNA, 19 nt, Artificial Sequence
  Description of Artificial Sequence Synthetic oligonucleotide
guguugucaauaaacauca

SEQ ID NO: 555 - RNA, 19 nt, Artificial Sequence
  Description of Artificial Sequence Synthetic oligonucleotide
uguucacucucacuaacuu

SEQ ID NO: 556 - RNA, 19 nt, Artificial Sequence
  Description of Artificial Sequence Synthetic oligonucleotide
aaguuagugagagugaaca

SEQ ID NO: 557 - RNA, 19 nt, Artificial Sequence
  Description of Artificial Sequence Synthetic oligonucleotide
ggacaaagaaagccgccuc

SEQ ID NO: 558 - RNA, 19 nt, Artificial Sequence
  Description of Artificial Sequence Synthetic oligonucleotide
gaggcggcuuucuuugucc

SEQ ID NO: 559 - RNA, 19 nt, Artificial Sequence
  Description of Artificial Sequence Synthetic oligonucleotide
caaccgcaguaauacggaa

SEQ ID NO: 560 - RNA, 19 nt, Artificial Sequence
  Description of Artificial Sequence Synthetic oligonucleotide
uuccguauuacugcgguug

SEQ ID NO: 561 - RNA, 19 nt, Artificial Sequence
  Description of Artificial Sequence Synthetic oligonucleotide
aagaaagccgccucaaacc

SEQ ID NO: 562 - RNA, 19 nt, Artificial Sequence
  Description of Artificial Sequence Synthetic oligonucleotide
gguuugaggcggcuuucuu

SEQ ID NO: 563 - RNA, 19 nt, Artificial Sequence
  Description of Artificial Sequence Synthetic oligonucleotide
aucucauauucuuucagaa

SEQ ID NO: 564 - RNA, 19 nt, Artificial Sequence
  Description of Artificial Sequence Synthetic oligonucleotide
uucugaaagaauaugagau

SEQ ID NO: 565 - RNA, 19 nt, Artificial Sequence
  Description of Artificial Sequence Synthetic oligonucleotide
auaacagaauacccgaggc

SEQ ID NO: 566 - RNA, 19 nt, Artificial Sequence
  Description of Artificial Sequence Synthetic oligonucleotide
gccucggguauucuguuau

SEQ ID NO: 567 - RNA, 19 nt, Artificial Sequence
  Description of Artificial Sequence Synthetic oligonucleotide
gccuguguucgaggauaug

SEQ ID NO: 568 - RNA, 19 nt, Artificial Sequence
  Description of Artificial Sequence Synthetic oligonucleotide
cauauccucgaacacaggc

SEQ ID NO: 569 - RNA, 19 nt, Artificial Sequence
  Description of Artificial Sequence Synthetic oligonucleotide
uuauugacaacacgcuuua

SEQ ID NO: 570 - RNA, 19 nt, Artificial Sequence
  Description of Artificial Sequence Synthetic oligonucleotide
uaaagcguguugucaauaa

SEQ ID NO: 571 - RNA, 19 nt, Artificial Sequence
  Description of Artificial Sequence Synthetic oligonucleotide
acucucacuaacuuacauc

SEQ ID NO: 572 - RNA, 19 nt, Artificial Sequence
  Description of Artificial Sequence Synthetic oligonucleotide
gauguaaguuagugagagu

SEQ ID NO: 573 - RNA, 19 nt, Artificial Sequence
  Description of Artificial Sequence Synthetic oligonucleotide
cugcaugauuuauagagua

SEQ ID NO: 574 - RNA, 19 nt, Artificial Sequence
  Description of Artificial Sequence Synthetic oligonucleotide
uacucuauaaaucaugcag

SEQ ID NO: 575 - RNA, 19 nt, Artificial Sequence
  Description of Artificial Sequence Synthetic oligonucleotide
ccgcaguaauacggaauau

SEQ ID NO: 576 - RNA, 19 nt, Artificial Sequence
  Description of Artificial Sequence Synthetic oligonucleotide
auauuccguauuacugcgg

SEQ ID NO: 577 - RNA, 19 nt, Artificial Sequence
  Description of Artificial Sequence Synthetic oligonucleotide
acaugcgccuugugaugac

SEQ ID NO: 578 - RNA, 19 nt, Artificial Sequence
  Description of Artificial Sequence Synthetic oligonucleotide
gucaucacaaggcgcaugu

SEQ ID NO: 579 - RNA, 19 nt, Artificial Sequence
  Description of Artificial Sequence Synthetic oligonucleotide
uuccauaacagaauacccg

SEQ ID NO: 580 - RNA, 19 nt, Artificial Sequence
  Description of Artificial Sequence Synthetic oligonucleotide
cggguauucuguuauggaa

SEQ ID NO: 581 - RNA, 19 nt, Artificial Sequence
  Description of Artificial Sequence Synthetic oligonucleotide
auacaaagugaucucauau

SEQ ID NO: 582 - RNA, 19 nt, Artificial Sequence
  Description of Artificial Sequence Synthetic oligonucleotide
auaugagaucacuuuguau

SEQ ID NO: 583 - RNA, 19 nt, Artificial Sequence
  Description of Artificial Sequence Synthetic oligonucleotide
cagaagacucuugcgucaa

SEQ ID NO: 584 - RNA, 19 nt, Artificial Sequence
  Description of Artificial Sequence Synthetic oligonucleotide
uugacgcaagagucuucug

SEQ ID NO: 585 - RNA, 19 nt, Artificial Sequence
  Description of Artificial Sequence Synthetic oligonucleotide
uuccagaaagaugauuagc

SEQ ID NO: 586 - RNA, 19 nt, Artificial Sequence
  Description of Artificial Sequence Synthetic oligonucleotide
gcuaaucaucuuucuggaa

SEQ ID NO: 587 - RNA, 19 nt, Artificial Sequence
  Description of Artificial Sequence Synthetic oligonucleotide
uaugacugauauugaucaa

SEQ ID NO: 588 - RNA, 19 nt, Artificial Sequence
  Description of Artificial Sequence Synthetic oligonucleotide
uugaucaauaucagucaua

SEQ ID NO: 589 - RNA, 19 nt, Artificial Sequence
  Description of Artificial Sequence Synthetic oligonucleotide
ggcugcaugauuuauagag

SEQ ID NO: 590 - RNA, 19 nt, Artificial Sequence
  Description of Artificial Sequence Synthetic oligonucleotide
cucuauaaaucaugcagcc

SEQ ID NO: 591 - RNA, 19 nt, Artificial Sequence
  Description of Artificial Sequence Synthetic oligonucleotide
ccgacuucacuguacaacc

SEQ ID NO: 592 - RNA, 19 nt, Artificial Sequence
  Description of Artificial Sequence Synthetic oligonucleotide
gguuguacagugaagucgg

SEQ ID NO: 593 - RNA, 19 nt, Artificial Sequence
  Description of Artificial Sequence Synthetic oligonucleotide
auaguaaaaugucuacccu

SEQ ID NO: 594 - RNA, 19 nt, Artificial Sequence
  Description of Artificial Sequence Synthetic oligonucleotide
aggguagacauuuuacuau

SEQ ID NO: 595 - RNA, 19 nt, Artificial Sequence
  Description of Artificial Sequence Synthetic oligonucleotide
ugacugauauugaucaaag

SEQ ID NO: 596 - RNA, 19 nt, Artificial Sequence
  Description of Artificial Sequence Synthetic oligonucleotide
cuuugaucaauaucaguca

SEQ ID NO: 597 - RNA, 19 nt, Artificial Sequence
  Description of Artificial Sequence Synthetic oligonucleotide
cuugugaugaccucgccug

SEQ ID NO: 598 - RNA, 19 nt, Artificial Sequence
  Description of Artificial Sequence Synthetic oligonucleotide
caggcgaggucaucacaag

SEQ ID NO: 599 - RNA, 19 nt, Artificial Sequence
  Description of Artificial Sequence Synthetic oligonucleotide
ucgaggauaugacugauau

SEQ ID NO: 600 - RNA, 19 nt, Artificial Sequence
  Description of Artificial Sequence Synthetic oligonucleotide
auaucagucauauccucga

SEQ ID NO: 601 - RNA, 19 nt, Artificial Sequence
  Description of Artificial Sequence Synthetic oligonucleotide
gaugaccucgccuguauuu

SEQ ID NO: 602 - RNA, 19 nt, Artificial Sequence
  Description of Artificial Sequence Synthetic oligonucleotide
aaauacaggcgaggucauc

SEQ ID NO: 603 - RNA, 19 nt, Artificial Sequence
  Description of Artificial Sequence Synthetic oligonucleotide
agaguaaacacguuuauuu

SEQ ID NO: 604 - RNA, 19 nt, Artificial Sequence
  Description of Artificial Sequence Synthetic oligonucleotide
aaauaaacguguuuacucu

SEQ ID NO: 605 - RNA, 19 nt, Artificial Sequence
  Description of Artificial Sequence Synthetic oligonucleotide
ccucucugugaaggauagu

SEQ ID NO: 606 - RNA, 19 nt, Artificial Sequence
  Description of Artificial Sequence Synthetic oligonucleotide
acuauccuucacagagagg

SEQ ID NO: 607 - RNA, 19 nt, Artificial Sequence
  Description of Artificial Sequence Synthetic oligonucleotide
augauguuuauugacaaca

SEQ ID NO: 608 - RNA, 19 nt, Artificial Sequence
  Description of Artificial Sequence Synthetic oligonucleotide
uguugucaauaaacaucau

SEQ ID NO: 609 - RNA, 19 nt, Artificial Sequence
  Description of Artificial Sequence Synthetic oligonucleotide
agacaacuuuggccgacuu

SEQ ID NO: 610 - RNA, 19 nt, Artificial Sequence
  Description of Artificial Sequence Synthetic oligonucleotide
aagucggccaaaguugucu

SEQ ID NO: 611 - RNA, 19 nt, Artificial Sequence
  Description of Artificial Sequence Synthetic oligonucleotide
uccauaacagaauacccga

SEQ ID NO: 612 - RNA, 19 nt, Artificial Sequence
  Description of Artificial Sequence Synthetic oligonucleotide
ucggguauucuguuaugga

SEQ ID NO: 613 - RNA, 19 nt, Artificial Sequence
  Description of Artificial Sequence Synthetic oligonucleotide
guacaaccgcaguaauacg

SEQ ID NO: 614 - RNA, 19 nt, Artificial Sequence
  Description of Artificial Sequence Synthetic oligonucleotide
cguauuacugcgguuguac

SEQ ID NO: 615 - RNA, 19 nt, Artificial Sequence
  Description of Artificial Sequence Synthetic oligonucleotide
ugauuagcacacaugcgcc

SEQ ID NO: 616 - RNA, 19 nt, Artificial Sequence
  Description of Artificial Sequence Synthetic oligonucleotide
ggcgcaugugugcuaauca

SEQ ID NO: 617 - RNA, 19 nt, Artificial Sequence
  Description of Artificial Sequence Synthetic oligonucleotide
augauuagcacacaugcgc

SEQ ID NO: 618 - RNA, 19 nt, Artificial Sequence
  Description of Artificial Sequence Synthetic oligonucleotide
gcgcaugugugcuaaucau

SEQ ID NO: 619 - RNA, 19 nt, Artificial Sequence
  Description of Artificial Sequence Synthetic oligonucleotide
gcuuuacuuuauaccugaa

SEQ ID NO: 620 - RNA, 19 nt, Artificial Sequence
  Description of Artificial Sequence Synthetic oligonucleotide
uucagguauaaaguaaagc

SEQ ID NO: 621 - RNA, 19 nt, Artificial Sequence
  Description of Artificial Sequence Synthetic oligonucleotide
ugcaugauuuauagaguaa

SEQ ID NO: 622 - RNA, 19 nt, Artificial Sequence
  Description of Artificial Sequence Synthetic oligonucleotide
uuacucuauaaaucaugca

SEQ ID NO: 623 - RNA, 19 nt, Artificial Sequence
  Description of Artificial Sequence Synthetic oligonucleotide
cuuuggccgacuucacugu

SEQ ID NO: 624 - RNA, 19 nt, Artificial Sequence
  Description of Artificial Sequence Synthetic oligonucleotide
acagugaagucggccaaag

SEQ ID NO: 625 - RNA, 19 nt, Artificial Sequence
  Description of Artificial Sequence Synthetic oligonucleotide
auccaccugaaaauauuga

SEQ ID NO: 626 - RNA, 19 nt, Artificial Sequence
  Description of Artificial Sequence Synthetic oligonucleotide
ucaauauuuucagguggau

SEQ ID NO: 627 - RNA, 19 nt, Artificial Sequence
  Description of Artificial Sequence Synthetic oligonucleotide
uguaauguucacucucacu

SEQ ID NO: 628 - RNA, 19 nt, Artificial Sequence
  Description of Artificial Sequence Synthetic oligonucleotide
agugagagugaacauuaca

SEQ ID NO: 629 - RNA, 19 nt, Artificial Sequence
  Description of Artificial Sequence Synthetic oligonucleotide
caccugaaaauauugauga

SEQ ID NO: 630 - RNA, 19 nt, Artificial Sequence
  Description of Artificial Sequence Synthetic oligonucleotide
ucaucaauauuuucaggug

SEQ ID NO: 631 - RNA, 19 nt, Artificial Sequence
  Description of Artificial Sequence Synthetic oligonucleotide
gacuucacuguacaaccgc

SEQ ID NO: 632 - RNA, 19 nt, Artificial Sequence
  Description of Artificial Sequence Synthetic oligonucleotide
gcgguuguacagugaaguc

SEQ ID NO: 633 - RNA, 19 nt, Artificial Sequence
  Description of Artificial Sequence Synthetic oligonucleotide
uaacagaauacccgaggcu

SEQ ID NO: 634 - RNA, 19 nt, Artificial Sequence
  Description of Artificial Sequence Synthetic oligonucleotide
agccucggguauucuguua

SEQ ID NO: 635 - RNA, 19 nt, Artificial Sequence
  Description of Artificial Sequence Synthetic oligonucleotide
aguaagaggacuggcugug

SEQ ID NO: 636 - RNA, 19 nt, Artificial Sequence
  Description of Artificial Sequence Synthetic oligonucleotide
cacagccaguccucuuacu

SEQ ID NO: 637 - RNA, 19 nt, Artificial Sequence
  Description of Artificial Sequence Synthetic oligonucleotide
aaccgcaguaauacggaau

SEQ ID NO: 638 - RNA, 19 nt, Artificial Sequence
  Description of Artificial Sequence Synthetic oligonucleotide
auuccguauuacugcgguu

SEQ ID NO: 639 - RNA, 19 nt, Artificial Sequence
  Description of Artificial Sequence Synthetic oligonucleotide
ugucuacccucuccuguaa

SEQ ID NO: 640 - RNA, 19 nt, Artificial Sequence
  Description of Artificial Sequence Synthetic oligonucleotide
uuacaggagaggguagaca

SEQ ID NO: 641 - RNA, 19 nt, Artificial Sequence
  Description of Artificial Sequence Synthetic oligonucleotide
gacaacuuuggccgacuuc

SEQ ID NO: 642 - RNA, 19 nt, Artificial Sequence
  Description of Artificial Sequence Synthetic oligonucleotide
gaagucggccaaaguuguc

SEQ ID NO: 643 - RNA, 19 nt, Artificial Sequence
  Description of Artificial Sequence Synthetic oligonucleotide
gaauacccgaggcugcaug

SEQ ID NO: 644 - RNA, 19 nt, Artificial Sequence
  Description of Artificial Sequence Synthetic oligonucleotide
caugcagccucggguauuc

SEQ ID NO: 645 - RNA, 19 nt, Artificial Sequence
  Description of Artificial Sequence Synthetic oligonucleotide
acuuacaucaaaguuaggu

SEQ ID NO: 646 - RNA, 19 nt, Artificial Sequence
  Description of Artificial Sequence Synthetic oligonucleotide
accuaacuuugauguaagu

SEQ ID NO: 647 - RNA, 19 nt, Artificial Sequence
  Description of Artificial Sequence Synthetic oligonucleotide
gauuagcacacaugcgccu

SEQ ID NO: 648 - RNA, 19 nt, Artificial Sequence
  Description of Artificial Sequence Synthetic oligonucleotide
aggcgcaugugugcuaauc

SEQ ID NO: 649 - RNA, 19 nt, Artificial Sequence
  Description of Artificial Sequence Synthetic oligonucleotide
uuacaucaaaguuaggugg

SEQ ID NO: 650 - RNA, 19 nt, Artificial Sequence
  Description of Artificial Sequence Synthetic oligonucleotide
ccaccuaacuuugauguaa

SEQ ID NO: 651 - DNA, 21 bp, Artificial Sequence
  Description of Artificial Sequence Synthetic oligonucleotide
uguucgaggauaugacugatt

SEQ ID NO: 652 - DNA, 21 bp, Artificial Sequence
  Description of Artificial Sequence Synthetic oligonucleotide
ucagucauauccucgaacatt

SEQ ID NO: 653 - DNA, 21 bp, Artificial Sequence
  Description of Artificial Sequence Synthetic oligonucleotide
uucacuguacaaccgcagutt

SEQ ID NO: 654 - DNA, 21 bp, Artificial Sequence
  Description of Artificial Sequence Synthetic oligonucleotide
acugcgguuguacagugaatt

SEQ ID NO: 655 - DNA, 21 bp, Artificial Sequence
  Description of Artificial Sequence Synthetic oligonucleotide
ucucuucguugacaaaagatt

SEQ ID NO: 656 - DNA, 21 bp, Artificial Sequence
  Description of Artificial Sequence Synthetic oligonucleotide
ucuuuugucaacgaagagatt

SEQ ID NO: 657 - DNA, 21 bp, Artificial Sequence
  Description of Artificial Sequence Synthetic oligonucleotide
uaguaaaaugucuacccuctt

SEQ ID NO: 658 - DNA, 21 bp, Artificial Sequence
  Description of Artificial Sequence Synthetic oligonucleotide
gaggguagacauuuuacuatt

SEQ ID NO: 659 - DNA, 21 bp, Artificial Sequence
  Description of Artificial Sequence Synthetic oligonucleotide
ucagaagacucuugcgucatt

SEQ ID NO: 660 - DNA, 21 bp, Artificial Sequence
  Description of Artificial Sequence Synthetic oligonucleotide
ugacgcaagagucuucugatt

SEQ ID NO: 661 - DNA, 21 bp, Artificial Sequence
  Description of Artificial Sequence Synthetic oligonucleotide
cgcuuuacuuuauaccugatt

SEQ ID NO: 662 - DNA, 21 bp, Artificial Sequence
  Description of Artificial Sequence Synthetic oligonucleotide
ucagguauaaaguaaagcgtt

SEQ ID NO: 663 - DNA, 21 bp, Artificial Sequence
  Description of Artificial Sequence Synthetic oligonucleotide
accagacugauaauauacatt

SEQ ID NO: 664 - DNA, 21 bp, Artificial Sequence
  Description of Artificial Sequence Synthetic oligonucleotide
uguauauuaucagucuggutt

SEQ ID NO: 665 - DNA, 21 bp, Artificial Sequence
  Description of Artificial Sequence Synthetic oligonucleotide
aaagacagccuguguucgatt

SEQ ID NO: 666 - DNA, 21 bp, Artificial Sequence
  Description of Artificial Sequence Synthetic oligonucleotide
ucgaacacaggcugucuuutt

SEQ ID NO: 667 - DNA, 21 bp, Artificial Sequence
  Description of Artificial Sequence Synthetic oligonucleotide
cugugaaggauaguaaaautt

SEQ ID NO: 668 - DNA, 21 bp, Artificial Sequence
  Description of Artificial Sequence Synthetic oligonucleotide
auuuuacuauccuucacagtt

SEQ ID NO: 669 - DNA, 21 bp, Artificial Sequence
  Description of Artificial Sequence Synthetic oligonucleotide
uuuauugacaacacgcuuutt

SEQ ID NO: 670 - DNA, 21 bp, Artificial Sequence
  Description of Artificial Sequence Synthetic oligonucleotide
aaagcguguugucaauaaatt

SEQ ID NO: 671 - DNA, 21 bp, Artificial Sequence
  Description of Artificial Sequence Synthetic oligonucleotide
ccauaacagaauacccgagtt

SEQ ID NO: 672 - DNA, 21 bp, Artificial Sequence
  Description of Artificial Sequence Synthetic oligonucleotide
cucggguauucuguuauggtt

SEQ ID NO: 673 - DNA, 21 bp, Artificial Sequence
  Description of Artificial Sequence Synthetic oligonucleotide
ucaaggaaaugauguuuautt

SEQ ID NO: 674 - DNA, 21 bp, Artificial Sequence
  Description of Artificial Sequence Synthetic oligonucleotide
auaaacaucauuuccuugatt

SEQ ID NO: 675 - DNA, 21 bp, Artificial Sequence
  Description of Artificial Sequence Synthetic oligonucleotide
acuucacuguacaaccgcatt

SEQ ID NO: 676 - DNA, 21 bp, Artificial Sequence
  Description of Artificial Sequence Synthetic oligonucleotide
ugcgguuguacagugaagutt

SEQ ID NO: 677 - DNA, 21 bp, Artificial Sequence
  Description of Artificial Sequence Synthetic oligonucleotide
accgcaguaauacggaauatt

SEQ ID NO: 678 - DNA, 21 bp, Artificial Sequence
  Description of Artificial Sequence Synthetic oligonucleotide
uauuccguauuacugcggutt

SEQ ID NO: 679 - DNA, 21 bp, Artificial Sequence
  Description of Artificial Sequence Synthetic oligonucleotide
aaagugaucucauauucuutt

SEQ ID NO: 680 - DNA, 21 bp, Artificial Sequence
  Description of Artificial Sequence Synthetic oligonucleotide
aagaauaugagaucacuuutt

SEQ ID NO: 681 - DNA, 21 bp, Artificial Sequence
  Description of Artificial Sequence Synthetic oligonucleotide
aggaagaugaugcuuucaatt

SEQ ID NO: 682 - DNA, 21 bp, Artificial Sequence
  Description of Artificial Sequence Synthetic oligonucleotide
uugaaagcaucaucuuccutt

SEQ ID NO: 683 - DNA, 21 bp, Artificial Sequence
  Description of Artificial Sequence Synthetic oligonucleotide
caaagaaagccgccucaaatt

SEQ ID NO: 684 - DNA, 21 bp, Artificial Sequence
  Description of Artificial Sequence Synthetic oligonucleotide
uuugaggcggcuuucuuugtt

SEQ ID NO: 685 - DNA, 21 bp, Artificial Sequence
  Description of Artificial Sequence Synthetic oligonucleotide
ugauguuuauugacaacactt

SEQ ID NO: 686 - DNA, 21 bp, Artificial Sequence
  Description of Artificial Sequence Synthetic oligonucleotide
guguugucaauaaacaucatt

SEQ ID NO: 687 - DNA, 21 bp, Artificial Sequence
  Description of Artificial Sequence Synthetic oligonucleotide
uguucacucucacuaacuutt

SEQ ID NO: 688 - DNA, 21 bp, Artificial Sequence
  Description of Artificial Sequence Synthetic oligonucleotide
aaguuagugagagugaacatt

SEQ ID NO: 689 - DNA, 21 bp, Artificial Sequence
  Description of Artificial Sequence Synthetic oligonucleotide
ggacaaagaaagccgccuctt

SEQ ID NO: 690 - DNA, 21 bp, Artificial Sequence
  Description of Artificial Sequence Synthetic oligonucleotide
gaggcggcuuucuuugucctt

SEQ ID NO: 691 - DNA, 21 bp, Artificial Sequence
  Description of Artificial Sequence Synthetic oligonucleotide
caaccgcaguaauacggaatt

SEQ ID NO: 692 - DNA, 21 bp, Artificial Sequence
  Description of Artificial Sequence Synthetic oligonucleotide
uuccguauuacugcgguugtt

SEQ ID NO: 693 - DNA, 21 bp, Artificial Sequence
  Description of Artificial Sequence Synthetic oligonucleotide
aagaaagccgccucaaacctt

SEQ ID NO: 694 - DNA, 21 bp, Artificial Sequence
  Description of Artificial Sequence Synthetic oligonucleotide
gguuugaggcggcuuucuutt

SEQ ID NO: 695 - DNA, 21 bp, Artificial Sequence
  Description of Artificial Sequence Synthetic oligonucleotide
aucucauauucuuucagaatt

SEQ ID NO: 696 - DNA, 21 bp, Artificial Sequence
  Description of Artificial Sequence Synthetic oligonucleotide
uucugaaagaauaugagautt

SEQ ID NO: 697 - DNA, 21 bp, Artificial Sequence
  Description of Artificial Sequence Synthetic oligonucleotide
auaacagaauacccgaggctt

SEQ ID NO: 698 - DNA, 21 bp, Artificial Sequence
  Description of Artificial Sequence Synthetic oligonucleotide
gccucggguauucuguuautt

SEQ ID NO: 699 - DNA, 21 bp, Artificial Sequence
  Description of Artificial Sequence Synthetic oligonucleotide
gccuguguucgaggauaugtt

SEQ ID NO: 700 - DNA, 21 bp, Artificial Sequence
  Description of Artificial Sequence Synthetic oligonucleotide
cauauccucgaacacaggctt

SEQ ID NO: 701 - DNA, 21 bp, Artificial Sequence
  Description of Artificial Sequence Synthetic oligonucleotide
uuauugacaacacgcuuuatt

SEQ ID NO: 702 - DNA, 21 bp, Artificial Sequence
  Description of Artificial Sequence Synthetic oligonucleotide
uaaagcguguugucaauaatt

SEQ ID NO: 703 - DNA, 21 bp, Artificial Sequence
  Description of Artificial Sequence Synthetic oligonucleotide
acucucacuaacuuacauctt

SEQ ID NO: 704 - DNA, 21 bp, Artificial Sequence
  Description of Artificial Sequence Synthetic oligonucleotide
gauguaaguuagugagagutt

SEQ ID NO: 705 - DNA, 21 bp, Artificial Sequence
  Description of Artificial Sequence Synthetic oligonucleotide
cugcaugauuuauagaguatt

SEQ ID NO: 706 - DNA, 21 bp, Artificial Sequence
  Description of Artificial Sequence Synthetic oligonucleotide
uacucuauaaaucaugcagtt

SEQ ID NO: 707 - DNA, 21 bp, Artificial Sequence
  Description of Artificial Sequence Synthetic oligonucleotide
ccgcaguaauacggaauautt

SEQ ID NO: 708 - DNA, 21 bp, Artificial Sequence
  Description of Artificial Sequence Synthetic oligonucleotide
auauuccguauuacugcggtt

SEQ ID NO: 709 - DNA, 21 bp, Artificial Sequence
  Description of Artificial Sequence Synthetic oligonucleotide
acaugcgccuugugaugactt

SEQ ID NO: 710 - DNA, 21 bp, Artificial Sequence
  Description of Artificial Sequence Synthetic oligonucleotide
gucaucacaaggcgcaugutt

SEQ ID NO: 711 - DNA, 21 bp, Artificial Sequence
  Description of Artificial Sequence Synthetic oligonucleotide
uuccauaacagaauacccgtt

SEQ ID NO: 712 - DNA, 21 bp, Artificial Sequence
  Description of Artificial Sequence Synthetic oligonucleotide
cggguauucuguuauggaatt

SEQ ID NO: 713 - DNA, 21 bp, Artificial Sequence
  Description of Artificial Sequence Synthetic oligonucleotide
auacaaagugaucucauautt

SEQ ID NO: 714 - DNA, 21 bp, Artificial Sequence
  Description of Artificial Sequence Synthetic oligonucleotide
auaugagaucacuuuguautt

SEQ ID NO: 715 - DNA, 21 bp, Artificial Sequence
  Description of Artificial Sequence Synthetic oligonucleotide
cagaagacucuugcgucaatt

SEQ ID NO: 716 - DNA, 21 bp, Artificial Sequence
  Description of Artificial Sequence Synthetic oligonucleotide
uugacgcaagagucuucugtt

SEQ ID NO: 717 - DNA, 21 bp, Artificial Sequence
  Description of Artificial Sequence Synthetic oligonucleotide
uuccagaaagaugauuagctt

SEQ ID NO: 718 - DNA, 21 bp, Artificial Sequence
  Description of Artificial Sequence Synthetic oligonucleotide
gcuaaucaucuuucuggaatt

SEQ ID NO: 719 - DNA, 21 bp, Artificial Sequence
  Description of Artificial Sequence Synthetic oligonucleotide
uaugacugauauugaucaatt

SEQ ID NO: 720 - DNA, 21 bp, Artificial Sequence
  Description of Artificial Sequence Synthetic oligonucleotide
uugaucaauaucagucauatt

SEQ ID NO: 721 - DNA, 21 bp, Artificial Sequence
  Description of Artificial Sequence Synthetic oligonucleotide
ggcugcaugauuuauagagtt

SEQ ID NO: 722 - DNA, 21 bp, Artificial Sequence
  Description of Artificial Sequence Synthetic oligonucleotide
cucuauaaaucaugcagcctt

SEQ ID NO: 723 - DNA, 21 bp, Artificial Sequence
  Description of Artificial Sequence Synthetic oligonucleotide
ccgacuucacuguacaacctt

SEQ ID NO: 724 - DNA, 21 bp, Artificial Sequence
  Description of Artificial Sequence Synthetic oligonucleotide
gguuguacagugaagucggtt

SEQ ID NO: 725 - DNA, 21 bp, Artificial Sequence
  Description of Artificial Sequence Synthetic oligonucleotide
auaguaaaaugucuacccutt

SEQ ID NO: 726 - DNA, 21 bp, Artificial Sequence
  Description of Artificial Sequence Synthetic oligonucleotide
aggguagacauuuuacuautt

SEQ ID NO: 727 - DNA, 21 bp, Artificial Sequence
  Description of Artificial Sequence Synthetic oligonucleotide
ugacugauauugaucaaagtt

SEQ ID NO: 728 - DNA, 21 bp, Artificial Sequence
  Description of Artificial Sequence Synthetic oligonucleotide
cuuugaucaauaucagucatt

SEQ ID NO: 729 - DNA, 21 bp, Artificial Sequence
  Description of Artificial Sequence Synthetic oligonucleotide
cuugugaugaccucgccugtt

SEQ ID NO: 730 - DNA, 21 bp, Artificial Sequence
  Description of Artificial Sequence Synthetic oligonucleotide
caggcgaggucaucacaagtt

SEQ ID NO: 731 - DNA, 21 bp, Artificial Sequence
  Description of Artificial Sequence Synthetic oligonucleotide
ucgaggauaugacugauautt

SEQ ID NO: 732 - DNA, 21 bp, Artificial Sequence
  Description of Artificial Sequence Synthetic oligonucleotide
auaucagucauauccucgatt

SEQ ID NO: 733 - DNA, 21 bp, Artificial Sequence
  Description of Artificial Sequence Synthetic oligonucleotide
gaugaccucgccuguauuutt

SEQ ID NO: 734 - DNA, 21 bp, Artificial Sequence
  Description of Artificial Sequence Synthetic oligonucleotide
aaauacaggcgaggucauctt

SEQ ID NO: 735 - DNA, 21 bp, Artificial Sequence
  Description of Artificial Sequence Synthetic oligonucleotide
agaguaaacacguuuauuutt

SEQ ID NO: 736 - DNA, 21 bp, Artificial Sequence
  Description of Artificial Sequence Synthetic oligonucleotide
aaauaaacguguuuacucutt

SEQ ID NO: 737 - DNA, 21 bp, Artificial Sequence
  Description of Artificial Sequence Synthetic oligonucleotide
ccucucugugaaggauagutt

SEQ ID NO: 738 - DNA, 21 bp, Artificial Sequence
  Description of Artificial Sequence Synthetic oligonucleotide
acuauccuucacagagaggtt

SEQ ID NO: 739 - DNA, 21 bp, Artificial Sequence
  Description of Artificial Sequence Synthetic oligonucleotide
augauguuuauugacaacatt

SEQ ID NO: 740 - DNA, 21 bp, Artificial Sequence
  Description of Artificial Sequence Synthetic oligonucleotide
uguugucaauaaacaucautt

SEQ ID NO: 741 - DNA, 21 bp, Artificial Sequence
  Description of Artificial Sequence Synthetic oligonucleotide
agacaacuuuggccgacuutt

SEQ ID NO: 742 - DNA, 21 bp, Artificial Sequence
  Description of Artificial Sequence Synthetic oligonucleotide
aagucggccaaaguugucutt

SEQ ID NO: 743 - DNA, 21 bp, Artificial Sequence
  Description of Artificial Sequence Synthetic oligonucleotide
uccauaacagaauacccgatt

SEQ ID NO: 744 - DNA, 21 bp, Artificial Sequence
  Description of Artificial Sequence Synthetic oligonucleotide
ucggguauucuguuauggatt

SEQ ID NO: 745 - DNA, 21 bp, Artificial Sequence
  Description of Artificial Sequence Synthetic oligonucleotide
guacaaccgcaguaauacgtt

SEQ ID NO: 746 - DNA, 21 bp, Artificial Sequence
  Description of Artificial Sequence Synthetic oligonucleotide
cguauuacugcgguuguactt

SEQ ID NO: 747 - DNA, 21 bp, Artificial Sequence
  Description of Artificial Sequence Synthetic oligonucleotide
ugauuagcacacaugcgcctt

SEQ ID NO: 748 - DNA, 21 bp, Artificial Sequence
  Description of Artificial Sequence Synthetic oligonucleotide
ggcgcaugugugcuaaucatt

SEQ ID NO: 749 - DNA, 21 bp, Artificial Sequence
  Description of Artificial Sequence Synthetic oligonucleotide
augauuagcacacaugcgctt

SEQ ID NO: 750 - DNA, 21 bp, Artificial Sequence
  Description of Artificial Sequence Synthetic oligonucleotide
gcgcaugugugcuaaucautt

SEQ ID NO: 751 - DNA, 21 bp, Artificial Sequence
  Description of Artificial Sequence Synthetic oligonucleotide
gcuuuacuuuauaccugaatt

SEQ ID NO: 752 - DNA, 21 bp, Artificial Sequence
  Description of Artificial Sequence Synthetic oligonucleotide
uucagguauaaaguaaagctt

SEQ ID NO: 753 - DNA, 21 bp, Artificial Sequence
  Description of Artificial Sequence Synthetic oligonucleotide
ugcaugauuuauagaguaatt

SEQ ID NO: 754 - DNA, 21 bp, Artificial Sequence
  Description of Artificial Sequence Synthetic oligonucleotide
uuacucuauaaaucaugcatt

SEQ ID NO: 755 - DNA, 21 bp, Artificial Sequence
  Description of Artificial Sequence Synthetic oligonucleotide
cuuuggccgacuucacugutt

SEQ ID NO: 756 - DNA, 21 bp, Artificial Sequence
  Description of Artificial Sequence Synthetic oligonucleotide
acagugaagucggccaaagtt

SEQ ID NO: 757 - DNA, 21 bp, Artificial Sequence
  Description of Artificial Sequence Synthetic oligonucleotide
auccaccugaaaauauugatt

SEQ ID NO: 758 - DNA, 21 bp, Artificial Sequence
  Description of Artificial Sequence Synthetic oligonucleotide
ucaauauuuucagguggautt

SEQ ID NO: 759 - DNA, 21 bp, Artificial Sequence
  Description of Artificial Sequence Synthetic oligonucleotide
uguaauguucacucucacutt

SEQ ID NO: 760 - DNA, 21 bp, Artificial Sequence
  Description of Artificial Sequence Synthetic oligonucleotide
agugagagugaacauuacatt

SEQ ID NO: 761 - DNA, 21 bp, Artificial Sequence
  Description of Artificial Sequence Synthetic oligonucleotide
caccugaaaauauugaugatt

SEQ ID NO: 762 - DNA, 21 bp, Artificial Sequence
  Description of Artificial Sequence Synthetic oligonucleotide
ucaucaauauuuucaggugtt

SEQ ID NO: 763 - DNA, 21 bp, Artificial Sequence
  Description of Artificial Sequence Synthetic oligonucleotide
gacuucacuguacaaccgctt

SEQ ID NO: 764 - DNA, 21 bp, Artificial Sequence
  Description of Artificial Sequence Synthetic oligonucleotide
gcgguuguacagugaaguctt

SEQ ID NO: 765 - DNA, 21 bp, Artificial Sequence
  Description of Artificial Sequence Synthetic oligonucleotide
uaacagaauacccgaggcutt

SEQ ID NO: 766 - DNA, 21 bp, Artificial Sequence
  Description of Artificial Sequence Synthetic oligonucleotide
agccucggguauucuguuatt

SEQ ID NO: 767 - DNA, 21 bp, Artificial Sequence
  Description of Artificial Sequence Synthetic oligonucleotide
aguaagaggacuggcugugtt

SEQ ID NO: 768 - DNA, 21 bp, Artificial Sequence
  Description of Artificial Sequence Synthetic oligonucleotide
cacagccaguccucuuacutt

SEQ ID NO: 769 - DNA, 21 bp, Artificial Sequence
  Description of Artificial Sequence Synthetic oligonucleotide
aaccgcaguaauacggaautt

SEQ ID NO: 770 - DNA, 21 bp, Artificial Sequence
  Description of Artificial Sequence Synthetic oligonucleotide
auuccguauuacugcgguutt

SEQ ID NO: 771 - DNA, 21 bp, Artificial Sequence
  Description of Artificial Sequence Synthetic oligonucleotide
ugucuacccucuccuguaatt

SEQ ID NO: 772 - DNA, 21 bp, Artificial Sequence
  Description of Artificial Sequence Synthetic oligonucleotide
uuacaggagaggguagacatt

SEQ ID NO: 773 - DNA, 21 bp, Artificial Sequence
  Description of Artificial Sequence Synthetic oligonucleotide
gacaacuuuggccgacuuctt

SEQ ID NO: 774 - DNA, 21 bp, Artificial Sequence
  Description of Artificial Sequence Synthetic oligonucleotide
gaagucggccaaaguuguctt

SEQ ID NO: 775 - DNA, 21 bp, Artificial Sequence
  Description of Artificial Sequence Synthetic oligonucleotide
gaauacccgaggcugcaugtt

SEQ ID NO: 776 - DNA, 21 bp, Artificial Sequence
  Description of Artificial Sequence Synthetic oligonucleotide
caugcagccucggguauuctt

SEQ ID NO: 777 - DNA, 21 bp, Artificial Sequence
  Description of Artificial Sequence Synthetic oligonucleotide
acuuacaucaaaguuaggutt

SEQ ID NO: 778 - DNA, 21 bp, Artificial Sequence
  Description of Artificial Sequence Synthetic oligonucleotide
accuaacuuugauguaagutt

SEQ ID NO: 779 - DNA, 21 bp, Artificial Sequence
  Description of Artificial Sequence Synthetic oligonucleotide
gauuagcacacaugcgccutt

SEQ ID NO: 780 - DNA, 21 bp, Artificial Sequence
  Description of Artificial Sequence Synthetic oligonucleotide
aggcgcaugugugcuaauctt

SEQ ID NO: 781 - DNA, 21 bp, Artificial Sequence
  Description of Artificial Sequence Synthetic oligonucleotide
uuacaucaaaguuagguggtt

SEQ ID NO: 782 - DNA, 21 bp, Artificial Sequence
  Description of Artificial Sequence Synthetic oligonucleotide
ccaccuaacuuugauguaatt

SEQ ID NO: 783 - DNA, 1145 bp, Homo spaiens
attctctccccagcttgctgagccctttgctcccctggcgactgcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatacaaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtg

SEQ ID NO: 784 - DNA, 1161 bp, Macaca fascicularis
  modified_base (204)..(223): a, c, t, g, unknown or other
attctctcccgagcctgctgagccctttgctcccctggcgactgcctggacaggcagcaaggagttgtctcccagtgcgttttgccctcctagctgccaactctggctgctaaagcggctgccacctgctacagtctgttacacagcctcgggaagaggaaagggacctcggaccttccagattgctttctctagcaagaaacnnnnnnnnnnnnnnnnnnnnatggctgctgaaccagcagaagacaattgcatcaattttgtggcaatgaaatttattgacagtacgctttactttatagctgaagatgatgaaaacctggaatcagattactttggcaagcttgaatctaaattatcaatcataagaaatttgaatgaccaagttctcttcattgaccaaggaaatcggcccctatttgaagatatgactgattctgactgtagagataatgcaccccggaccatatttattataaatatgtataaagatagccagcctagaggtatggctgtagccatctctgtgaaatgtgagaaaatttcaactctctcctgtgagaacagaattatttcctttaaggaaatgaatcctcctgataacatcaaggatacgaaaagtgacatcatattctttcagagaagtgtcccaggacatgataataagatgcaatttgaatcttcatcatacgaaggatactttctagcttgtgaaaaagagagagacctttataaactcattttgaaaaaaaaggatgaattgggggatagatctataatgttcactgttcaaaacgaagactagctattaaaatttcatgcygggcrcrgtggctcamgcctgtaatcccagcnactttgggaggcygagrygggygrkmwcmygagkkwrrgagttcaagaccwgcctgrccaacatrrtgaaaccygtctctaytaaaaatacaaaaanttagctgggcgtggtggtggnntcgcctatnaatcccagctacttgggaggctgaggcagragaatyacattrmrctccgagaggcagaggttgtggtgagccgagatcatgcncactgcactccagcctgggyracarcagcragactyyrtctynnnnnnnnnnnnnnnnnnnnnnnnnnnnnnnnnnnnnnnnnnnnnnnnnnnnn

SEQ ID NO: 785 - DNA, 865 bp, Mus musculus
ggcacagctggacctggtgggggttctctgtggttccatgctttctggactcctgcctgctggctggagctgctgacaggcctgacatcttctgcaacctccagcatcaggacaaagaaagccgcctcaaaccttccaaatcacttcctcttggcccaggaacaatggctgccatgtcagaagactcttgcgtcaacttcaaggaaatgatgtttattgacaacacgctttactttatacctgaagaaaatggagacctggaatcagacaactttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacatgtacaaagacagtgaagtaagaggactggctgtgaccctctctgtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagttaggtggggagggtttgtgttccagaaagatgattagcacacatgcgccttgtgatgacctcgcctgtatttccataacagaatacccgaggctgcatgatttatagagtaaacacgtttatttg

SEQ ID NO: 786 - DNA, 42 bp, Artificial Sequence
  Description of Artificial Sequence Synthetic probe
ccgctttagcagccagagttgtttttctcttggaaagaaagt

SEQ ID NO: 787 - DNA, 41 bp, Artificial Sequence
  Description of Artificial Sequence Synthetic probe
tagactgcagcaggtggcagtttttctcttggaaagaaagt

SEQ ID NO: 788 - DNA, 44 bp, Artificial Sequence
  Description of Artificial Sequence Synthetic probe
gatctggaaggtctgaggttccttttttctcttggaaagaaagt

SEQ ID NO: 789 - DNA, 44 bp, Artificial Sequence
  Description of Artificial Sequence Synthetic probe
aatagtttgttgcgagaggaagctttttctcttggaaagaaagt

SEQ ID NO: 790 - DNA, 46 bp, Artificial Sequence
  Description of Artificial Sequence Synthetic probe
acaggagagagttgaaattttctcatttttctcttggaaagaaagt

SEQ ID NO: 791 - DNA, 47 bp, Artificial Sequence
  Description of Artificial Sequence Synthetic probe
tccttaaaggaaataattttgttctctttttctcttggaaagaaagt

SEQ ID NO: 792 - DNA, 44 bp, Artificial Sequence
  Description of Artificial Sequence Synthetic probe
gctgactgtccaggcagtcgtttttaggcataggacccgtgtct

SEQ ID NO: 793 - DNA, 45 bp, Artificial Sequence
  Description of Artificial Sequence Synthetic probe
tgcactgggagacaattcctttttttaggcataggacccgtgtct

SEQ ID NO: 794 - DNA, 48 bp, Artificial Sequence
  Description of Artificial Sequence Synthetic probe
caattgtcttctactggttcagcatttttaggcataggacccgtgtct

SEQ ID NO: 795 - DNA, 48 bp, Artificial Sequence
  Description of Artificial Sequence Synthetic probe
gattccaggttttcatcatcttcatttttaggcataggacccgtgtct

SEQ ID NO: 796 - DNA, 47 bp, Artificial Sequence
  Description of Artificial Sequence Synthetic probe
atcttcaaatagaggccgatttctttttaggcataggacccgtgtct

SEQ ID NO: 797 - DNA, 51 bp, Artificial Sequence
  Description of Artificial Sequence Synthetic probe
cacttcacagagatagttacagccatatttttaggcataggacccgtgtct

SEQ ID NO: 798 - DNA, 49 bp, Artificial Sequence
  Description of Artificial Sequence Synthetic probe
cttgatgttatcaggaggattcatttttttaggcataggacccgtgtct

SEQ ID NO: 799 - DNA, 18 bp, Artificial Sequence
  Description of Artificial Sequence Synthetic probe
gcagccaggagggcaaaa

SEQ ID NO: 800 - DNA, 20 bp, Artificial Sequence
  Description of Artificial Sequence Synthetic probe
ttcctcttcccgaagctgtg

SEQ ID NO: 801 - DNA, 21 bp, Artificial Sequence
  Description of Artificial Sequence Synthetic probe
gccatctttattcctgcgaca

SEQ ID NO: 802 - DNA, 23 bp, Artificial Sequence
  Description of Artificial Sequence Synthetic probe
atttcattgccacaaagttgatg

SEQ ID NO: 803 - DNA, 28 bp, Artificial Sequence
  Description of Artificial Sequence Synthetic probe
gctataaagtaaagcgtattgtcaataa

SEQ ID NO: 804 - DNA, 24 bp, Artificial Sequence
  Description of Artificial Sequence Synthetic probe
gattcaagcttgccaaagtaatct

SEQ ID NO: 805 - DNA, 29 bp, Artificial Sequence
  Description of Artificial Sequence Synthetic probe
cattcaaatttcttatgactgataattta

SEQ ID NO: 806 - DNA, 24 bp, Artificial Sequence
  Description of Artificial Sequence Synthetic probe
cttggtcaatgaagagaacttggt

SEQ ID NO: 807 - DNA, 29 bp, Artificial Sequence
  Description of Artificial Sequence Synthetic probe
gcattatctctacagtcagaatcagtcat

SEQ ID NO: 808 - DNA, 24 bp, Artificial Sequence
  Description of Artificial Sequence Synthetic probe
cttataataaatatggtccggggt

SEQ ID NO: 809 - DNA, 27 bp, Artificial Sequence
  Description of Artificial Sequence Synthetic probe
cctctaggctggctatctttatacata

SEQ ID NO: 810 - DNA, 28 bp, Artificial Sequence
  Description of Artificial Sequence Synthetic probe
gaaagaatatgatgtcactttttgtatc

SEQ ID NO: 811 - DNA, 41 bp, Artificial Sequence
  Description of Artificial Sequence Synthetic probe
gaatttgccatgggtggaattttttctcttggaaagaaagt

SEQ ID NO: 812 - DNA, 41 bp, Artificial Sequence
  Description of Artificial Sequence Synthetic probe
ggagggatctcgctcctggatttttctcttggaaagaaagt

SEQ ID NO: 813 - DNA, 40 bp, Artificial Sequence
  Description of Artificial Sequence Synthetic probe
ccccagccttctccatggttttttctcttggaaagaaagt

SEQ ID NO: 814 - DNA, 40 bp, Artificial Sequence
  Description of Artificial Sequence Synthetic probe
gctcccccctgcaaatgagtttttctcttggaaagaaagt

SEQ ID NO: 815 - DNA, 42 bp, Artificial Sequence
  Description of Artificial Sequence Synthetic probe
agccttgacggtgccatgtttttaggcataggacccgtgtct

SEQ ID NO: 816 - DNA, 45 bp, Artificial Sequence
  Description of Artificial Sequence Synthetic probe
gatgacaagcttcccgttctctttttaggcataggacccgtgtct

SEQ ID NO: 817 - DNA, 46 bp, Artificial Sequence
  Description of Artificial Sequence Synthetic probe
agatggtgatgggatttccatttttttaggcataggacccgtgtct

SEQ ID NO: 818 - DNA, 44 bp, Artificial Sequence
  Description of Artificial Sequence Synthetic probe
gcatcgccccacttgatttttttttaggcataggacccgtgtct

SEQ ID NO: 819 - DNA, 43 bp, Artificial Sequence
  Description of Artificial Sequence Synthetic probe
cacgacgtactcagcgccatttttaggcataggacccgtgtct

SEQ ID NO: 820 - DNA, 46 bp, Artificial Sequence
  Description of Artificial Sequence Synthetic probe
ggcagagatgatgacccttttgtttttaggcataggacccgtgtct

SEQ ID NO: 821 - DNA, 21 bp, Artificial Sequence
  Description of Artificial Sequence Synthetic probe
ggtgaagacgccagtggactc

SEQ ID NO: 822 - DNA, 38 bp, Artificial Sequence
  Description of Artificial Sequence Synthetic probe
ccagccagcaggcaggatttttctcttggaaagaaagt

SEQ ID NO: 823 - DNA, 39 bp, Artificial Sequence
  Description of Artificial Sequence Synthetic probe
tgtcaggcctgtcagcagcttttttgaagttaccgtttt

SEQ ID NO: 824 - DNA, 40 bp, Artificial Sequence
  Description of Artificial Sequence Synthetic probe
gatgctggaggttgcagaagatttttctgagtcaaagcat

SEQ ID NO: 825 - DNA, 41 bp, Artificial Sequence
  Description of Artificial Sequence Synthetic probe
gaggcggctttctttgtccttttttctcttggaaagaaagt

SEQ ID NO: 826 - DNA, 45 bp, Artificial Sequence
  Description of Artificial Sequence Synthetic probe
aagaggaagtgatttggaaggttttttttctcttggaaagaaagt

SEQ ID NO: 827 - DNA, 39 bp, Artificial Sequence
  Description of Artificial Sequence Synthetic probe
agccattgttcctgggcctttttctcttggaaagaaagt

SEQ ID NO: 828 - DNA, 41 bp, Artificial Sequence
  Description of Artificial Sequence Synthetic probe
cgcaagagtcttctgacatggctttttgaagttaccgtttt

SEQ ID NO: 829 - DNA, 45 bp, Artificial Sequence
  Description of Artificial Sequence Synthetic probe
aataaacatcatttccttgaagttgatttttctgagtcaaagcat

SEQ ID NO: 830 - DNA, 45 bp, Artificial Sequence
  Description of Artificial Sequence Synthetic probe
tcaggtataaagtaaagcgtgttgtctttttgaagttaccgtttt

SEQ ID NO: 831 - DNA, 42 bp, Artificial Sequence
  Description of Artificial Sequence Synthetic probe
ctgattccaggtctccattttcttttttctgagtcaaagcat

SEQ ID NO: 832 - DNA, 22 bp, Artificial Sequence
  Description of Artificial Sequence Synthetic probe
cagtgaagtcggccaaagttgt

SEQ ID NO: 833 - DNA, 43 bp, Artificial Sequence
  Description of Artificial Sequence Synthetic probe
atattccgtattactgcggttgtatttttgaagttaccgtttt

SEQ ID NO: 834 - DNA, 43 bp, Artificial Sequence
  Description of Artificial Sequence Synthetic probe
tcaacgaagagaacttggtcattttttttctgagtcaaagcat

SEQ ID NO: 835 - DNA, 43 bp, Artificial Sequence
  Description of Artificial Sequence Synthetic probe
ctcgaacacaggctgtcttttgtttttctcttggaaagaaagt

SEQ ID NO: 836 - DNA, 46 bp, Artificial Sequence
  Description of Artificial Sequence Synthetic probe
ggcactttgatcaatatcagtcatatctttttgaagttaccgtttt

SEQ ID NO: 837 - DNA, 41 bp, Artificial Sequence
  Description of Artificial Sequence Synthetic probe
tcagtctggtctggggttcacttttttctgagtcaaagcat

SEQ ID NO: 838 - DNA, 31 bp, Artificial Sequence
  Description of Artificial Sequence Synthetic probe
cttacttcactgtctttgtacatgtatatta

SEQ ID NO: 839 - DNA, 40 bp, Artificial Sequence
  Description of Artificial Sequence Synthetic probe
gagagggtcacagccagtccttttttgaagttaccgtttt

SEQ ID NO: 840 - DNA, 45 bp, Artificial Sequence
  Description of Artificial Sequence Synthetic probe
gggtagacattttactatccttcacatttttctgagtcaaagcat

SEQ ID NO: 841 - DNA, 47 bp, Artificial Sequence
  Description of Artificial Sequence Synthetic probe
ggaaatgatcttgttcttacaggagatttttctcttggaaagaaagt

SEQ ID NO: 842 - DNA, 40 bp, Artificial Sequence
  Description of Artificial Sequence Synthetic probe
cgaggctggcactgcacaatttttctcttggaaagaaagt

SEQ ID NO: 843 - DNA, 41 bp, Artificial Sequence
  Description of Artificial Sequence Synthetic probe
cttcaccattttgtctacgggatttttgaagttaccgtttt

SEQ ID NO: 844 - DNA, 39 bp, Artificial Sequence
  Description of Artificial Sequence Synthetic probe
ccaaatccgttcacaccgactttttctgagtcaaagcat

SEQ ID NO: 845 - DNA, 38 bp, Artificial Sequence
  Description of Artificial Sequence Synthetic probe
ccaggcgcccaatacggtttttctcttggaaagaaagt

SEQ ID NO: 846 - DNA, 38 bp, Artificial Sequence
  Description of Artificial Sequence Synthetic probe
caaatggcagccctggtgatttttgaagttaccgtttt

SEQ ID NO: 847 - DNA, 41 bp, Artificial Sequence
  Description of Artificial Sequence Synthetic probe
aacaatctccactttgccactgtttttctgagtcaaagcat

SEQ ID NO: 848 - DNA, 19 bp, Artificial Sequence
  Description of Artificial Sequence Synthetic probe
tgaaggggtcgttgatggc

SEQ ID NO: 849 - DNA, 26 bp, Artificial Sequence
  Description of Artificial Sequence Synthetic probe
catgtagaccatgtagttgaggtcaa

SEQ ID NO: 850 - DNA, 44 bp, Artificial Sequence
  Description of Artificial Sequence Synthetic probe
ccgtgagtggagtcatactggaatttttctcttggaaagaaagt

SEQ ID NO: 851 - DNA, 42 bp, Artificial Sequence
  Description of Artificial Sequence Synthetic probe
ttgactgtgccgttgaatttgtttttctcttggaaagaaagt

SEQ ID NO: 852 - DNA, 37 bp, Artificial Sequence
  Description of Artificial Sequence Synthetic probe
agcttcccattctcggcctttttgaagttaccgtttt

SEQ ID NO: 853 - DNA, 38 bp, Artificial Sequence
  Description of Artificial Sequence Synthetic probe
gggcttcccgttgatgacatttttctgagtcaaagcat

SEQ ID NO: 854 - DNA, 41 bp, Artificial Sequence
  Description of Artificial Sequence Synthetic probe
cgctcctggaagatggtgattttttctcttggaaagaaagt

SEQ ID NO: 855 - DNA, 23 bp, Artificial Sequence
  Description of Artificial Sequence Synthetic probe
cccatttgatgttagtggggtct

SEQ ID NO: 856 - DNA, 41 bp, Artificial Sequence
  Description of Artificial Sequence Synthetic probe
atactcagcaccggcctcactttttctcttggaaagaaagt

SEQ ID NO: 857 - DNA, 21 bp, Artificial Sequence
  Description of Artificial Sequence Synthetic oligonucleotide
gaaacacuuugccgcaggctt

SEQ ID NO: 858 - DNA, 21 bp, Artificial Sequence
  Description of Artificial Sequence Synthetic oligonucleotide
gccugcggcaaaguguuuctt

SEQ ID NO: 859 - DNA, 21 bp, Artificial Sequence
  Description of Artificial Sequence Synthetic oligonucleotide
gaaauaauuuggcgcaggctt

SEQ ID NO: 860 - DNA, 21 bp, Artificial Sequence
  Description of Artificial Sequence Synthetic oligonucleotide
gccugcgccaaauuauuuctt

SEQ ID NO: 861 - DNA, 21 bp, Artificial Sequence
  Description of Artificial Sequence Synthetic oligonucleotide
cagacuauuuaccgcagggtt

SEQ ID NO: 862 - DNA, 21 bp, Artificial Sequence
  Description of Artificial Sequence Synthetic oligonucleotide
cccugcgguaaauagucugtt

SEQ ID NO: 863 - DNA, 21 bp, Artificial Sequence
  Description of Artificial Sequence Synthetic oligonucleotide
gaaacucuuuguggcaggatt

SEQ ID NO: 864 - DNA, 21 bp, Artificial Sequence
  Description of Artificial Sequence Synthetic oligonucleotide
uccugccacaaagaguuuctt

SEQ ID NO: 865 - DNA, 21 bp, Artificial Sequence
  Description of Artificial Sequence Synthetic oligonucleotide
gaaacuuuuugucacaggutt

SEQ ID NO: 866 - DNA, 21 bp, Artificial Sequence
  Description of Artificial Sequence Synthetic oligonucleotide
accugugacaaaaaguuuctt

SEQ ID NO: 867 - DNA, 21 bp, Artificial Sequence
  Description of Artificial Sequence Synthetic oligonucleotide
aaagcaauuuguugcaggctt

SEQ ID NO: 868 - DNA, 21 bp, Artificial Sequence
  Description of Artificial Sequence Synthetic oligonucleotide
gccugcaacaaauugcuuutt

SEQ ID NO: 869 - DNA, 21 bp, Artificial Sequence
  Description of Artificial Sequence Synthetic oligonucleotide
uaaagaauuugucacaggatt

SEQ ID NO: 870 - DNA, 21 bp, Artificial Sequence
  Description of Artificial Sequence Synthetic oligonucleotide
uccugugacaaauucuuuatt

SEQ ID NO: 871 - DNA, 21 bp, Artificial Sequence
  Description of Artificial Sequence Synthetic oligonucleotide
uaaucuacuuguugcaggatt

SEQ ID NO: 872 - DNA, 21 bp, Artificial Sequence
  Description of Artificial Sequence Synthetic oligonucleotide
uccugcaacaaguagauuatt

SEQ ID NO: 873 - DNA, 21 bp, Artificial Sequence
  Description of Artificial Sequence Synthetic oligonucleotide
aaaaccauugguugcaggatt

SEQ ID NO: 874 - DNA, 21 bp, Artificial Sequence
  Description of Artificial Sequence Synthetic oligonucleotide
uccugcaaccaaugguuuutt

SEQ ID NO: 875 - DNA, 21 bp, Artificial Sequence
  Description of Artificial Sequence Synthetic oligonucleotide
gaaacuaaguguggcagggtt

SEQ ID NO: 876 - DNA, 21 bp, Artificial Sequence
  Description of Artificial Sequence Synthetic oligonucleotide
cccugccacacuuaguuuctt

SEQ ID NO: 877 - DNA, 21 bp, Artificial Sequence
  Description of Artificial Sequence Synthetic oligonucleotide
caaaguauuuggcgcuggctt

SEQ ID NO: 878 - DNA, 21 bp, Artificial Sequence
  Description of Artificial Sequence Synthetic oligonucleotide
gccagcgccaaauacuuugtt

SEQ ID NO: 879 - DNA, 21 bp, Artificial Sequence
  Description of Artificial Sequence Synthetic oligonucleotide
cauacuauuugacgcagaatt

SEQ ID NO: 880 - DNA, 21 bp, Artificial Sequence
  Description of Artificial Sequence Synthetic oligonucleotide
uucugcgucaaauaguaugtt

SEQ ID NO: 881 - DNA, 21 bp, Artificial Sequence
  Description of Artificial Sequence Synthetic oligonucleotide
uccuaaaacaaauaguuuatt

SEQ ID NO: 882 - DNA, 21 bp, Artificial Sequence
  Description of Artificial Sequence Synthetic oligonucleotide
uaaacuauuuguuuuaggatt

SEQ ID NO: 883 - DNA, 21 bp, Artificial Sequence
  Description of Artificial Sequence Synthetic oligonucleotide
uccuguaacaacuaguuuutt

SEQ ID NO: 884 - DNA, 21 bp, Artificial Sequence
  Description of Artificial Sequence Synthetic oligonucleotide
aaaacuaguuguuacaggatt

SEQ ID NO: 885 - DNA, 21 bp, Artificial Sequence
  Description of Artificial Sequence Synthetic oligonucleotide
aaauauaaaaagauagccgtt

SEQ ID NO: 886 - DNA, 21 bp, Artificial Sequence
  Description of Artificial Sequence Synthetic oligonucleotide
cggcuaucuuuuuauauuutt

SEQ ID NO: 887 - DNA, 21 bp, Artificial Sequence
  Description of Artificial Sequence Synthetic oligonucleotide
cuuuuaauaaagauagccatt

SEQ ID NO: 888 - DNA, 21 bp, Artificial Sequence
  Description of Artificial Sequence Synthetic oligonucleotide
uggcuaucuuuauuaaaagtt

SEQ ID NO: 889 - DNA, 21 bp, Artificial Sequence
  Description of Artificial Sequence Synthetic oligonucleotide
auauggaagaagauagccatt

SEQ ID NO: 890 - DNA, 21 bp, Artificial Sequence
  Description of Artificial Sequence Synthetic oligonucleotide
uggcuaucuucuuccauautt

SEQ ID NO: 891 - DNA, 21 bp, Artificial Sequence
  Description of Artificial Sequence Synthetic oligonucleotide
auauuucuuaagauagccctt

SEQ ID NO: 892 - DNA, 21 bp, Artificial Sequence
  Description of Artificial Sequence Synthetic oligonucleotide
gggcuaucuuaagaaauautt

SEQ ID NO: 893 - DNA, 21 bp, Artificial Sequence
  Description of Artificial Sequence Synthetic oligonucleotide
ucauguaacaagauagccatt

SEQ ID NO: 894 - DNA, 21 bp, Artificial Sequence
  Description of Artificial Sequence Synthetic oligonucleotide
uggcuaucuuguuacaugatt

SEQ ID NO: 895 - DNA, 21 bp, Artificial Sequence
  Description of Artificial Sequence Synthetic oligonucleotide
auauauauuaugauagccutt

SEQ ID NO: 896 - DNA, 21 bp, Artificial Sequence
  Description of Artificial Sequence Synthetic oligonucleotide
aggcuaucauaauauauautt

SEQ ID NO: 897 - DNA, 21 bp, Artificial Sequence
  Description of Artificial Sequence Synthetic oligonucleotide
guauuuagaaacauagccctt

SEQ ID NO: 898 - DNA, 21 bp, Artificial Sequence
  Description of Artificial Sequence Synthetic oligonucleotide
gggcuauguuucuaaauactt

SEQ ID NO: 899 - DNA, 21 bp, Artificial Sequence
  Description of Artificial Sequence Synthetic oligonucleotide
auaugcauaaagauagacutt

SEQ ID NO: 900 - DNA, 21 bp, Artificial Sequence
  Description of Artificial Sequence Synthetic oligonucleotide
agucuaucuuuaugcauautt

SEQ ID NO: 901 - DNA, 21 bp, Artificial Sequence
  Description of Artificial Sequence Synthetic oligonucleotide
auauguaaaaagauagcactt

SEQ ID NO: 902 - DNA, 21 bp, Artificial Sequence
  Description of Artificial Sequence Synthetic oligonucleotide
gugcuaucuuuuuacauautt

SEQ ID NO: 903 - DNA, 21 bp, Artificial Sequence
  Description of Artificial Sequence Synthetic oligonucleotide
uuauguauuaagauaaccutt

SEQ ID NO: 904 - DNA, 21 bp, Artificial Sequence
  Description of Artificial Sequence Synthetic oligonucleotide
agguuaucuuaauacauaatt

SEQ ID NO: 905 - DNA, 21 bp, Artificial Sequence
  Description of Artificial Sequence Synthetic oligonucleotide
guauguauaaggauggccatt

SEQ ID NO: 906 - DNA, 21 bp, Artificial Sequence
  Description of Artificial Sequence Synthetic oligonucleotide
uggccauccuuauacauactt

SEQ ID NO: 907 - DNA, 21 bp, Artificial Sequence
  Description of Artificial Sequence Synthetic oligonucleotide
auauguauaaagagagacatt

SEQ ID NO: 908 - DNA, 21 bp, Artificial Sequence
  Description of Artificial Sequence Synthetic oligonucleotide
ugucucucuuuauacauautt

SEQ ID NO: 909 - DNA, 21 bp, Artificial Sequence
  Description of Artificial Sequence Synthetic oligonucleotide
aggcuauuuuuauacuuaatt

SEQ ID NO: 910 - DNA, 21 bp, Artificial Sequence
  Description of Artificial Sequence Synthetic oligonucleotide
uuaaguauaaaaauagccutt

SEQ ID NO: 911 - DNA, 21 bp, Artificial Sequence
  Description of Artificial Sequence Synthetic oligonucleotide
aggcuaucucuauacacaatt

SEQ ID NO: 912 - DNA, 21 bp, Artificial Sequence
  Description of Artificial Sequence Synthetic oligonucleotide
uuguguauagagauagccutt

SEQ ID NO: 913 - DNA, 21 bp, Artificial Sequence
  Description of Artificial Sequence Synthetic oligonucleotide
caaaugacaugauaauaaatt

SEQ ID NO: 914 - DNA, 21 bp, Artificial Sequence
  Description of Artificial Sequence Synthetic oligonucleotide
uuuauuaucaugucauuugtt

SEQ ID NO: 915 - DNA, 21 bp, Artificial Sequence
  Description of Artificial Sequence Synthetic oligonucleotide
ccccagccaugauaauaaatt

SEQ ID NO: 916 - DNA, 21 bp, Artificial Sequence
  Description of Artificial Sequence Synthetic oligonucleotide
uuuauuaucauggcuggggtt

SEQ ID NO: 917 - DNA, 21 bp, Artificial Sequence
  Description of Artificial Sequence Synthetic oligonucleotide
cucaugacaggauaauaagtt

SEQ ID NO: 918 - DNA, 21 bp, Artificial Sequence
  Description of Artificial Sequence Synthetic oligonucleotide
cuuauuauccugucaugagtt

SEQ ID NO: 919 - DNA, 21 bp, Artificial Sequence
  Description of Artificial Sequence Synthetic oligonucleotide
ucaaggaacugauaauaagtt

SEQ ID NO: 920 - DNA, 21 bp, Artificial Sequence
  Description of Artificial Sequence Synthetic oligonucleotide
cuuauuaucaguuccuugatt

SEQ ID NO: 921 - DNA, 21 bp, Artificial Sequence
  Description of Artificial Sequence Synthetic oligonucleotide
uacaagacauguuaauaagtt

SEQ ID NO: 922 - DNA, 21 bp, Artificial Sequence
  Description of Artificial Sequence Synthetic oligonucleotide
cuuauuaacaugucuuguatt

SEQ ID NO: 923 - DNA, 21 bp, Artificial Sequence
  Description of Artificial Sequence Synthetic oligonucleotide
cccaggacauuaaaauaagtt

SEQ ID NO: 924 - DNA, 21 bp, Artificial Sequence
  Description of Artificial Sequence Synthetic oligonucleotide
cuuauuuuaauguccugggtt

SEQ ID NO: 925 - DNA, 21 bp, Artificial Sequence
  Description of Artificial Sequence Synthetic oligonucleotide
uccaggacaugguaauagutt

SEQ ID NO: 926 - DNA, 21 bp, Artificial Sequence
  Description of Artificial Sequence Synthetic oligonucleotide
acuauuaccauguccuggatt

SEQ ID NO: 927 - DNA, 21 bp, Artificial Sequence
  Description of Artificial Sequence Synthetic oligonucleotide
accaggacaugagaauagatt

SEQ ID NO: 928 - DNA, 21 bp, Artificial Sequence
  Description of Artificial Sequence Synthetic oligonucleotide
ucuauucucauguccuggutt

SEQ ID NO: 929 - DNA, 21 bp, Artificial Sequence
  Description of Artificial Sequence Synthetic oligonucleotide
accaggacaugauuauagctt

SEQ ID NO: 930 - DNA, 21 bp, Artificial Sequence
  Description of Artificial Sequence Synthetic oligonucleotide
gcuauaaucauguccuggutt

SEQ ID NO: 931 - DNA, 21 bp, Artificial Sequence
  Description of Artificial Sequence Synthetic oligonucleotide
cccaggacaugaaaaaaautt

SEQ ID NO: 932 - DNA, 21 bp, Artificial Sequence
  Description of Artificial Sequence Synthetic oligonucleotide
auuuuuuucauguccugggtt

SEQ ID NO: 933 - DNA, 21 bp, Artificial Sequence
  Description of Artificial Sequence Synthetic oligonucleotide
gccaggacaugaucaugactt

SEQ ID NO: 934 - DNA, 21 bp, Artificial Sequence
  Description of Artificial Sequence Synthetic oligonucleotide
gucaugaucauguccuggctt

SEQ ID NO: 935 - DNA, 21 bp, Artificial Sequence
  Description of Artificial Sequence Synthetic oligonucleotide
uccaggacaugauacugautt

SEQ ID NO: 936 - DNA, 21 bp, Artificial Sequence
  Description of Artificial Sequence Synthetic oligonucleotide
aucaguaucauguccuggatt

SEQ ID NO: 937 - DNA, 21 bp, Artificial Sequence
  Description of Artificial Sequence Synthetic oligonucleotide
auuauuaacauguccugggtt

SEQ ID NO: 938 - DNA, 21 bp, Artificial Sequence
  Description of Artificial Sequence Synthetic oligonucleotide
cccaggacauguuaauaautt

SEQ ID NO: 939 - DNA, 21 bp, Artificial Sequence
  Description of Artificial Sequence Synthetic oligonucleotide
cuuguugucauguccuggutt

SEQ ID NO: 940 - DNA, 21 bp, Artificial Sequence
  Description of Artificial Sequence Synthetic oligonucleotide
accaggacaugacaacaagtt

SEQ ID NO: 941 - DNA, 18 bp, Artificial Sequence
  Description of Artificial Sequence Synthetic probe
gcgcagtcggggcagtat

SEQ ID NO: 942 - DNA, 20 bp, Artificial Sequence
  Description of Artificial Sequence Synthetic probe
ccacgatgaagaagggcact

SEQ ID NO: 943 - DNA, 41 bp, Artificial Sequence
  Description of Artificial Sequence Synthetic probe
gggtcaggactccaagcagatttttctcttggaaagaaagt

SEQ ID NO: 944 - DNA, 41 bp, Artificial Sequence
  Description of Artificial Sequence Synthetic probe
cccattgtccagctctcccttttttctcttggaaagaaagt

SEQ ID NO: 945 - DNA, 41 bp, Artificial Sequence
  Description of Artificial Sequence Synthetic probe
gccaggtcgatgaaggtgattttttctcttggaaagaaagt

SEQ ID NO: 946 - DNA, 40 bp, Artificial Sequence
  Description of Artificial Sequence Synthetic probe
ggcactgacgaggagcaggtttttctcttggaaagaaagt

SEQ ID NO: 947 - DNA, 41 bp, Artificial Sequence
  Description of Artificial Sequence Synthetic probe
gtgccagcaatcccagtgtttttttctcttggaaagaaagt

SEQ ID NO: 948 - DNA, 43 bp, Artificial Sequence
  Description of Artificial Sequence Synthetic probe
gcacataggtcgatcttgctgatttttctcttggaaagaaagt

SEQ ID NO: 949 - DNA, 40 bp, Artificial Sequence
  Description of Artificial Sequence Synthetic probe
agccaggctgcttgaggactttttctcttggaaagaaagt

SEQ ID NO: 950 - DNA, 39 bp, Artificial Sequence
  Description of Artificial Sequence Synthetic probe
tgctgggcagcagtgacgtttttctcttggaaagaaagt

SEQ ID NO: 951 - DNA, 36 bp, Artificial Sequence
  Description of Artificial Sequence Synthetic probe
ccccaggacggccacactttttgaagttaccgtttt

SEQ ID NO: 952 - DNA, 42 bp, Artificial Sequence
  Description of Artificial Sequence Synthetic probe
gttgacttcccagagtccacatttttttctgagtcaaagcat

SEQ ID NO: 953 - DNA, 33 bp, Artificial Sequence
  Description of Artificial Sequence Synthetic probe
cgagcccggccccgtttttgaagttaccgtttt

SEQ ID NO: 954 - DNA, 38 bp, Artificial Sequence
  Description of Artificial Sequence Synthetic probe
gtggcggaaaaggttgagctttttctgagtcaaagcat

SEQ ID NO: 955 - DNA, 41 bp, Artificial Sequence
  Description of Artificial Sequence Synthetic probe
ggccagactgaatctcatgcagtttttgaagttaccgtttt

SEQ ID NO: 956 - DNA, 40 bp, Artificial Sequence
  Description of Artificial Sequence Synthetic probe
cgaagctgatgctggaggttctttttctgagtcaaagcat

SEQ ID NO: 957 - DNA, 40 bp, Artificial Sequence
  Description of Artificial Sequence Synthetic probe
tgctgttaaagcccaggatcttttttgaagttaccgtttt

SEQ ID NO: 958 - DNA, 42 bp, Artificial Sequence
  Description of Artificial Sequence Synthetic probe
gctgtaattcaccacctctcccttttttctgagtcaaagcat

SEQ ID NO: 959 - DNA, 41 bp, Artificial Sequence
  Description of Artificial Sequence Synthetic probe
tctcttctgctgtccgtgagtctttttgaagttaccgtttt

SEQ ID NO: 960 - DNA, 42 bp, Artificial Sequence
  Description of Artificial Sequence Synthetic probe
catcttggagctgctctcacagatttttctgagtcaaagcat

SEQ ID NO: 961 - DNA, 43 bp, Artificial Sequence
  Description of Artificial Sequence Synthetic probe
gtgtgtaggtacttatggtggccttttttgaagttaccgtttt

SEQ ID NO: 962 - DNA, 40 bp, Artificial Sequence
  Description of Artificial Sequence Synthetic probe
gatgtgaggccaaagatggtgtttttctgagtcaaagcat

SEQ ID NO: 963 - DNA, 39 bp, Artificial Sequence
  Description of Artificial Sequence Synthetic probe
gccccagatgttcccttgtgtttttgaagttaccgtttt

SEQ ID NO: 964 - DNA, 35 bp, Artificial Sequence
  Description of Artificial Sequence Synthetic probe
ttcagggccagggccatttttctgagtcaaagcat

SEQ ID NO: 965 - DNA, 41 bp, Artificial Sequence
  Description of Artificial Sequence Synthetic probe
tgtcctctccactgtggtcttgtttttgaagttaccgtttt

SEQ ID NO: 966 - DNA, 37 bp, Artificial Sequence
  Description of Artificial Sequence Synthetic probe
ccgctccagctggcgtactttttctgagtcaaagcat

SEQ ID NO: 967 - DNA, 37 bp, Artificial Sequence
  Description of Artificial Sequence Synthetic probe
gcatggggaccttgtggctttttgaagttaccgtttt

SEQ ID NO: 968 - DNA, 41 bp, Artificial Sequence
  Description of Artificial Sequence Synthetic probe
gcatcatcctcagaggtgaccatttttctgagtcaaagcat

SEQ ID NO: 969 - DNA, 21 bp, Artificial Sequence
  Description of Artificial Sequence Synthetic probe
tggtcctcgctcttgtcctct

SEQ ID NO: 970 - DNA, 21 bp, Artificial Sequence
  Description of Artificial Sequence Synthetic probe
tgcaccaggaccttgtcactt

SEQ ID NO: 971 - DNA, 44 bp, Artificial Sequence
  Description of Artificial Sequence Synthetic probe
acttgcttgcactcttgcagtagtttttctcttggaaagaaagt

SEQ ID NO: 972 - DNA, 41 bp, Artificial Sequence
  Description of Artificial Sequence Synthetic probe
ggagtaaagctctgcagcgctttttctcttggaaagaaagt

SEQ ID NO: 973 - DNA, 39 bp, Artificial Sequence
  Description of Artificial Sequence Synthetic probe
ttgtcctctgggcccacgtttttctcttggaaagaaagt

SEQ ID NO: 974 - DNA, 37 bp, Artificial Sequence
  Description of Artificial Sequence Synthetic probe
gccgcagccgcacagttttttctcttggaaagaaagt

SEQ ID NO: 975 - DNA, 44 bp, Artificial Sequence
  Description of Artificial Sequence Synthetic probe
acctgcagaagttttccagagactttttctcttggaaagaaagt

SEQ ID NO: 976 - DNA, 38 bp, Artificial Sequence
  Description of Artificial Sequence Synthetic probe
ccacctgcggggcaatttttttctcttggaaagaaagt

SEQ ID NO: 977 - DNA, 48 bp, Artificial Sequence
  Description of Artificial Sequence Synthetic probe
actgctcagaatatactctgcttttaatttttctcttggaaagaaagt

SEQ ID NO: 978 - DNA, 46 bp, Artificial Sequence
  Description of Artificial Sequence Synthetic probe
ccctctgatctgtatacagaccgactttttctcttggaaagaaagt

SEQ ID NO: 979 - DNA, 40 bp, Artificial Sequence
  Description of Artificial Sequence Synthetic probe
tccaacaagagctgatccagctttttgaagttaccgtttt

SEQ ID NO: 980 - DNA, 37 bp, Artificial Sequence
  Description of Artificial Sequence Synthetic probe
tccgacacatctggcgcttttttctgagtcaaagcat

SEQ ID NO: 981 - DNA, 39 bp, Artificial Sequence
  Description of Artificial Sequence Synthetic probe
cagggtccttaactcgctggtttttgaagttaccgtttt

SEQ ID NO: 982 - DNA, 40 bp, Artificial Sequence
  Description of Artificial Sequence Synthetic probe
catttcctttgcctcctggattttttctgagtcaaagcat

SEQ ID NO: 983 - DNA, 37 bp, Artificial Sequence
  Description of Artificial Sequence Synthetic probe
aggcacgaagggccactttttttgaagttaccgtttt

SEQ ID NO: 984 - DNA, 42 bp, Artificial Sequence
  Description of Artificial Sequence Synthetic probe
gcttgtttgtactgccacttttctttttctgagtcaaagcat

SEQ ID NO: 985 - DNA, 43 bp, Artificial Sequence
  Description of Artificial Sequence Synthetic probe
ccaatcacatccttcaggtttgtttttttgaagttaccgtttt

SEQ ID NO: 986 - DNA, 34 bp, Artificial Sequence
  Description of Artificial Sequence Synthetic probe
tgctgcaacccggcgtttttctgagtcaaagcat

SEQ ID NO: 987 - DNA, 39 bp, Artificial Sequence
  Description of Artificial Sequence Synthetic probe
ccctcagggacgccagtaactttttgaagttaccgtttt

SEQ ID NO: 988 - DNA, 37 bp, Artificial Sequence
  Description of Artificial Sequence Synthetic probe
cccgagcgaggatggaggtttttctgagtcaaagcat

SEQ ID NO: 989 - DNA, 42 bp, Artificial Sequence
  Description of Artificial Sequence Synthetic probe
ggtccaccaagaacacaatagcctttttgaagttaccgtttt

SEQ ID NO: 990 - DNA, 39 bp, Artificial Sequence
  Description of Artificial Sequence Synthetic probe
gtcgagcccatacaggaacctttttctgagtcaaagcat

SEQ ID NO: 991 - DNA, 39 bp, Artificial Sequence
  Description of Artificial Sequence Synthetic probe
acttgtggagacctttggcgtttttgaagttaccgtttt

SEQ ID NO: 992 - DNA, 35 bp, Artificial Sequence
  Description of Artificial Sequence Synthetic probe
ggcgtggctggctgcatttttctgagtcaaagcat

SEQ ID NO: 993 - DNA, 37 bp, Artificial Sequence
  Description of Artificial Sequence Synthetic probe
ttcgggcttggcgaataatttttgaagttaccgtttt

SEQ ID NO: 994 - DNA, 42 bp, Artificial Sequence
  Description of Artificial Sequence Synthetic probe
aagttttcctgagttcacggagatttttctgagtcaaagcat

SEQ ID NO: 995 - DNA, 43 bp, Artificial Sequence
  Description of Artificial Sequence Synthetic probe
cgttgctccattgttgcttattagtttttgaagttaccgtttt

SEQ ID NO: 996 - DNA, 42 bp, Artificial Sequence
  Description of Artificial Sequence Synthetic probe
tcatttctgtacagccaggtacctttttctgagtcaaagcat

SEQ ID NO: 997 - DNA, 31 bp, Artificial Sequence
  Description of Artificial Sequence Synthetic probe
tcttttttctagtagtattcgaaatagatta

SEQ ID NO: 998 - DNA, 25 bp, Artificial Sequence
  Description of Artificial Sequence Synthetic probe
gagagaagaagttatgtggctaagg

SEQ ID NO: 999 - DNA, 47 bp, Artificial Sequence
  Description of Artificial Sequence Synthetic probe
tttggctttttatatcagaagaaatttttttctcttggaaagaaagt

SEQ ID NO: 1000 - DNA, 42 bp, Artificial Sequence
  Description of Artificial Sequence Synthetic probe
tgcttgaagaggcaatttgcttttttctcttggaaagaaagt

SEQ ID NO: 1001 - DNA, 44 bp, Artificial Sequence
  Description of Artificial Sequence Synthetic probe
gcagagagcagtgtcaaacgatgtttttctcttggaaagaaagt

SEQ ID NO: 1002 - DNA, 43 bp, Artificial Sequence
  Description of Artificial Sequence Synthetic probe
tcttgcatgagtcttcgatccatttttctcttggaaagaaagt

SEQ ID NO: 1003 - DNA, 46 bp, Artificial Sequence
  Description of Artificial Sequence Synthetic probe
tttcttcttccgtattaacagcacttttttctcttggaaagaaagt

SEQ ID NO: 1004 - DNA, 46 bp, Artificial Sequence
  Description of Artificial Sequence Synthetic probe
agagaaggataactgaccgacttcttttttctcttggaaagaaagt

SEQ ID NO: 1005 - DNA, 43 bp, Artificial Sequence
  Description of Artificial Sequence Synthetic probe
agaattcaccttgcagctcaagtttttctcttggaaagaaagt

SEQ ID NO: 1006 - DNA, 43 bp, Artificial Sequence
  Description of Artificial Sequence Synthetic probe
tgcccacctttgactgtattgttttttctcttggaaagaaagt

SEQ ID NO: 1007 - DNA, 42 bp, Artificial Sequence
  Description of Artificial Sequence Synthetic probe
cttttcggatgtccacaatattgtttttgaagttaccgtttt

SEQ ID NO: 1008 - DNA, 44 bp, Artificial Sequence
  Description of Artificial Sequence Synthetic probe
ttcattgcaatctcacggttatatatttttctgagtcaaagcat

SEQ ID NO: 1009 - DNA, 47 bp, Artificial Sequence
  Description of Artificial Sequence Synthetic probe
tacccatttaggtcagtataaaatctattttttgaagttaccgtttt

SEQ ID NO: 1010 - DNA, 44 bp, Artificial Sequence
  Description of Artificial Sequence Synthetic probe
cagtgtcattctaggttgaatctggtttttctgagtcaaagcat

SEQ ID NO: 1011 - DNA, 43 bp, Artificial Sequence
  Description of Artificial Sequence Synthetic probe
cattgtggtcatgggatagacatttttttgaagttaccgtttt

SEQ ID NO: 1012 - DNA, 40 bp, Artificial Sequence
  Description of Artificial Sequence Synthetic probe
tttggcatcctggatataggctttttctgagtcaaagcat

SEQ ID NO: 1013 - DNA, 42 bp, Artificial Sequence
  Description of Artificial Sequence Synthetic probe
aactcgaaacccctaatgactgatttttgaagttaccgtttt

SEQ ID NO: 1014 - DNA, 46 bp, Artificial Sequence
  Description of Artificial Sequence Synthetic probe
tgataacttcaatctgaccactattcatttttctgagtcaaagcat

SEQ ID NO: 1015 - DNA, 40 bp, Artificial Sequence
  Description of Artificial Sequence Synthetic probe
tgctcaaggccacgattatcatttttgaagttaccgtttt

SEQ ID NO: 1016 - DNA, 46 bp, Artificial Sequence
  Description of Artificial Sequence Synthetic probe
gctgtaatcttgttatcctggataccttttttctgagtcaaagcat

SEQ ID NO: 1017 - DNA, 43 bp, Artificial Sequence
  Description of Artificial Sequence Synthetic probe
cattggaatgactggatgattcattttttgaagttaccgtttt

SEQ ID NO: 1018 - DNA, 43 bp, Artificial Sequence
  Description of Artificial Sequence Synthetic probe
ggtaggtgaggagaacttatttgctttttctgagtcaaagcat

SEQ ID NO: 1019 - DNA, 43 bp, Artificial Sequence
  Description of Artificial Sequence Synthetic probe
aaggcaaagatgactgtaatggagtttttgaagttaccgtttt

SEQ ID NO: 1020 - DNA, 45 bp, Artificial Sequence
  Description of Artificial Sequence Synthetic probe
tctcaaattaaccagatgaatgtcactttttctgagtcaaagcat

SEQ ID NO: 1021 - DNA, 38 bp, Artificial Sequence
  Description of Artificial Sequence Synthetic probe
gcctcattggagtgcccattttttgaagttaccgtttt

SEQ ID NO: 1022 - DNA, 41 bp, Artificial Sequence
  Description of Artificial Sequence Synthetic probe
ttttctgtggaggatcaaggcttttttctgagtcaaagcat

SEQ ID NO: 1023 - DNA, 16 bp, Artificial Sequence
  Description of Artificial Sequence Synthetic probe
aggggggccccacact

SEQ ID NO: 1024 - DNA, 20 bp, Artificial Sequence
  Description of Artificial Sequence Synthetic probe
gggactccttgttgctcagg

SEQ ID NO: 1025 - DNA, 40 bp, Artificial Sequence
  Description of Artificial Sequence Synthetic probe
ctcctcactccctggcccttttttctcttggaaagaaagt

SEQ ID NO: 1026 - DNA, 40 bp, Artificial Sequence
  Description of Artificial Sequence Synthetic probe
gaagcagggcgtccacttctttttctcttggaaagaaagt

SEQ ID NO: 1027 - DNA, 41 bp, Artificial Sequence
  Description of Artificial Sequence Synthetic probe
tgaccgttcattctgggctgtttttctcttggaaagaaagt

SEQ ID NO: 1028 - DNA, 39 bp, Artificial Sequence
  Description of Artificial Sequence Synthetic probe
gcaggcctcggctggagttttttctcttggaaagaaagt

SEQ ID NO: 1029 - DNA, 38 bp, Artificial Sequence
  Description of Artificial Sequence Synthetic probe
gggccgagggctaagcctttttctcttggaaagaaagt

SEQ ID NO: 1030 - DNA, 41 bp, Artificial Sequence
  Description of Artificial Sequence Synthetic probe
gctgttccggtaaggcacgttttttctcttggaaagaaagt

SEQ ID NO: 1031 - DNA, 43 bp, Artificial Sequence
  Description of Artificial Sequence Synthetic probe
tctgcagcaggtaggtcagttttttttctcttggaaagaaagt

SEQ ID NO: 1032 - DNA, 41 bp, Artificial Sequence
  Description of Artificial Sequence Synthetic probe
cactggttcaccttggaggctttttctcttggaaagaaagt

SEQ ID NO: 1033 - DNA, 41 bp, Artificial Sequence
  Description of Artificial Sequence Synthetic probe
atcgagcattggtgacagtgagtttttgaagttaccgtttt

SEQ ID NO: 1034 - DNA, 42 bp, Artificial Sequence
  Description of Artificial Sequence Synthetic probe
tttctcacaggagacagggacattttttctgagtcaaagcat

SEQ ID NO: 1035 - DNA, 37 bp, Artificial Sequence
  Description of Artificial Sequence Synthetic probe
gattctggcgggccagattttttgaagttaccgtttt

SEQ ID NO: 1036 - DNA, 34 bp, Artificial Sequence
  Description of Artificial Sequence Synthetic probe
tgcgggccacagccctttttctgagtcaaagcat

SEQ ID NO: 1037 - DNA, 40 bp, Artificial Sequence
  Description of Artificial Sequence Synthetic probe
gaatacactgtggctgcgtgatttttgaagttaccgtttt

SEQ ID NO: 1038 - DNA, 41 bp, Artificial Sequence
  Description of Artificial Sequence Synthetic probe
gctccccagaaatctgtagctgtttttctgagtcaaagcat

SEQ ID NO: 1039 - DNA, 39 bp, Artificial Sequence
  Description of Artificial Sequence Synthetic probe
cggccaggtccacaagactgtttttgaagttaccgtttt

SEQ ID NO: 1040 - DNA, 39 bp, Artificial Sequence
  Description of Artificial Sequence Synthetic probe
ggggtcaagtcgctcactcctttttctgagtcaaagcat

SEQ ID NO: 1041 - DNA, 34 bp, Artificial Sequence
  Description of Artificial Sequence Synthetic probe
cgttcccgctccccgtttttgaagttaccgtttt

SEQ ID NO: 1042 - DNA, 38 bp, Artificial Sequence
  Description of Artificial Sequence Synthetic probe
ggcctgtgtttcccgaaggtttttctgagtcaaagcat

SEQ ID NO: 1043 - DNA, 40 bp, Artificial Sequence
  Description of Artificial Sequence Synthetic probe
cgtggacaggctgctgttaattttttgaagttaccgtttt

SEQ ID NO: 1044 - DNA, 39 bp, Artificial Sequence
  Description of Artificial Sequence Synthetic probe
gccatgataaccagccccagtttttctgagtcaaagcat

SEQ ID NO: 1045 - DNA, 43 bp, Artificial Sequence
  Description of Artificial Sequence Synthetic probe
tcttagcactaccacccagagagttttttgaagttaccgtttt

SEQ ID NO: 1046 - DNA, 43 bp, Artificial Sequence
  Description of Artificial Sequence Synthetic probe
gagaaatgttcacaaacatgagcatttttctgagtcaaagcat

SEQ ID NO: 1047 - DNA, 40 bp, Artificial Sequence
  Description of Artificial Sequence Synthetic probe
cggagacgttctcttccagtgtttttgaagttaccgtttt

SEQ ID NO: 1048 - DNA, 42 bp, Artificial Sequence
  Description of Artificial Sequence Synthetic probe
aaagcgtagagagttgagggacttttttctgagtcaaagcat

SEQ ID NO: 1049 - DNA, 19 bp, Artificial Sequence
  Description of Artificial Sequence Synthetic probe
ccccctgcctgatagtccc

SEQ ID NO: 1050 - DNA, 20 bp, Artificial Sequence
  Description of Artificial Sequence Synthetic probe
ccctcagagttccttccccc

SEQ ID NO: 1051 - DNA, 23 bp, Artificial Sequence
  Description of Artificial Sequence Synthetic probe
tcccttttcatcattaacctgct

SEQ ID NO: 1052 - DNA, 22 bp, Artificial Sequence
  Description of Artificial Sequence Synthetic probe
cctgagagtccgccctattctc

SEQ ID NO: 1053 - DNA, 44 bp, Artificial Sequence
  Description of Artificial Sequence Synthetic probe
actttcagagccttcattctggttttttctcttggaaagaaagt

SEQ ID NO: 1054 - DNA, 40 bp, Artificial Sequence
  Description of Artificial Sequence Synthetic probe
gacctcctggggatgcaagtttttctcttggaaagaaagt

SEQ ID NO: 1055 - DNA, 44 bp, Artificial Sequence
  Description of Artificial Sequence Synthetic probe
tgttcctgtttttgcttctgttctttttctcttggaaagaaagt

SEQ ID NO: 1056 - DNA, 40 bp, Artificial Sequence
  Description of Artificial Sequence Synthetic probe
ccctctggctccccagtgatttttctcttggaaagaaagt

SEQ ID NO: 1057 - DNA, 41 bp, Artificial Sequence
  Description of Artificial Sequence Synthetic probe
ggacctccctgaccctgagctttttctcttggaaagaaagt

SEQ ID NO: 1058 - DNA, 45 bp, Artificial Sequence
  Description of Artificial Sequence Synthetic probe
ccaaaaaggtttgactcttttcgttttttctcttggaaagaaagt

SEQ ID NO: 1059 - DNA, 40 bp, Artificial Sequence
  Description of Artificial Sequence Synthetic probe
cgctgactggcaccccttttttttctcttggaaagaaagt

SEQ ID NO: 1060 - DNA, 39 bp, Artificial Sequence
  Description of Artificial Sequence Synthetic probe
gctctttcgcctcctgcctttttctcttggaaagaaagt

SEQ ID NO: 1061 - DNA, 44 bp, Artificial Sequence
  Description of Artificial Sequence Synthetic probe
gcagggagaataatatctggtctgatttttgaagttaccgtttt

SEQ ID NO: 1062 - DNA, 41 bp, Artificial Sequence
  Description of Artificial Sequence Synthetic probe
tggtctctgcaggatgctcttctttttctgagtcaaagcat

SEQ ID NO: 1063 - DNA, 39 bp, Artificial Sequence
  Description of Artificial Sequence Synthetic probe
ctcctcaggtggcagctctttttttgaagttaccgtttt

SEQ ID NO: 1064 - DNA, 39 bp, Artificial Sequence
  Description of Artificial Sequence Synthetic probe
agcccttgactttcctgcagtttttctgagtcaaagcat

SEQ ID NO: 1065 - DNA, 39 bp, Artificial Sequence
  Description of Artificial Sequence Synthetic probe
ccctgctcctccagaacttgtttttgaagttaccgtttt

SEQ ID NO: 1066 - DNA, 40 bp, Artificial Sequence
  Description of Artificial Sequence Synthetic probe
cgaaatcctgcttcctgctgttttttctgagtcaaagcat

SEQ ID NO: 1067 - DNA, 39 bp, Artificial Sequence
  Description of Artificial Sequence Synthetic probe
cccatcggcacaaacatccttttttgaagttaccgtttt

SEQ ID NO: 1068 - DNA, 44 bp, Artificial Sequence
  Description of Artificial Sequence Synthetic probe
ctatcatctgttcctcccctaatagtttttctgagtcaaagcat

SEQ ID NO: 1069 - DNA, 41 bp, Artificial Sequence
  Description of Artificial Sequence Synthetic probe
cccaagcatcacatcttgtacctttttgaagttaccgtttt

SEQ ID NO: 1070 - DNA, 41 bp, Artificial Sequence
  Description of Artificial Sequence Synthetic probe
gccccattctttctccttgtcttttttctgagtcaaagcat

SEQ ID NO: 1071 - DNA, 40 bp, Artificial Sequence
  Description of Artificial Sequence Synthetic probe
tcccactcaccgtcattcagatttttgaagttaccgtttt

SEQ ID NO: 1072 - DNA, 41 bp, Artificial Sequence
  Description of Artificial Sequence Synthetic probe
cttcctctccatatcctcctgctttttctgagtcaaagcat

SEQ ID NO: 1073 - DNA, 43 bp, Artificial Sequence
  Description of Artificial Sequence Synthetic probe
ctcttcctctcttctccctgttcttttttgaagttaccgtttt

SEQ ID NO: 1074 - DNA, 38 bp, Artificial Sequence
  Description of Artificial Sequence Synthetic probe
ttctggcacctgcagctcctttttctgagtcaaagcat

SEQ ID NO: 1075 - DNA, 44 bp, Artificial Sequence
  Description of Artificial Sequence Synthetic probe
tttccagttacttcctctgattttctttttgaagttaccgtttt

SEQ ID NO: 1076 - DNA, 44 bp, Artificial Sequence
  Description of Artificial Sequence Synthetic probe
ctcctttataccatgatcttcttgctttttctgagtcaaagcat

SEQ ID NO: 1077 - DNA, 19 bp, Artificial Sequence
  Description of Artificial Sequence Synthetic probe
ctcctgcacgtgggctctc

SEQ ID NO: 1078 - DNA, 23 bp, Artificial Sequence
  Description of Artificial Sequence Synthetic probe
aagtctctggaggaagcaaagaa

SEQ ID NO: 1079 - DNA, 18 bp, Artificial Sequence
  Description of Artificial Sequence Synthetic probe
tgcaccaagcccacagca

SEQ ID NO: 1080 - DNA, 45 bp, Artificial Sequence
  Description of Artificial Sequence Synthetic probe
cgaaataatcatctgtccctcctgtttttctcttggaaagaaagt

SEQ ID NO: 1081 - DNA, 39 bp, Artificial Sequence
  Description of Artificial Sequence Synthetic probe
gcacagcgccagcagaggtttttctcttggaaagaaagt

SEQ ID NO: 1082 - DNA, 44 bp, Artificial Sequence
  Description of Artificial Sequence Synthetic probe
ggtaaaatctccaggtaaggctttttttctcttggaaagaaagt

SEQ ID NO: 1083 - DNA, 44 bp, Artificial Sequence
  Description of Artificial Sequence Synthetic probe
aaagatttgggaagaaggttcactttttctcttggaaagaaagt

SEQ ID NO: 1084 - DNA, 42 bp, Artificial Sequence
  Description of Artificial Sequence Synthetic probe
tgctcttcagcctttgctttttttttctcttggaaagaaagt

SEQ ID NO: 1085 - DNA, 43 bp, Artificial Sequence
  Description of Artificial Sequence Synthetic probe
cagaggctgcagagaagagaaatttttctcttggaaagaaagt

SEQ ID NO: 1086 - DNA, 39 bp, Artificial Sequence
  Description of Artificial Sequence Synthetic probe
cggctctggtgttgacggtttttctcttggaaagaaagt

SEQ ID NO: 1087 - DNA, 40 bp, Artificial Sequence
  Description of Artificial Sequence Synthetic probe
tgcctgggcctcaggtacatttttctcttggaaagaaagt

SEQ ID NO: 1088 - DNA, 39 bp, Artificial Sequence
  Description of Artificial Sequence Synthetic probe
agcaggcaactctcaggcaatttttgaagttaccgtttt

SEQ ID NO: 1089 - DNA, 40 bp, Artificial Sequence
  Description of Artificial Sequence Synthetic probe
cgaatcactgtgtccgtggtgtttttctgagtcaaagcat

SEQ ID NO: 1090 - DNA, 44 bp, Artificial Sequence
  Description of Artificial Sequence Synthetic probe
agtaatgtatgcccctaagtagctttttttgaagttaccgtttt

SEQ ID NO: 1091 - DNA, 40 bp, Artificial Sequence
  Description of Artificial Sequence Synthetic probe
agatggaggaggcttccactttttttctgagtcaaagcat

SEQ ID NO: 1092 - DNA, 44 bp, Artificial Sequence
  Description of Artificial Sequence Synthetic probe
catgcttttctgaaactaaaaaggttttttgaagttaccgtttt

SEQ ID NO: 1093 - DNA, 39 bp, Artificial Sequence
  Description of Artificial Sequence Synthetic probe
ccaaagagatcggtgcccagtttttctgagtcaaagcat

SEQ ID NO: 1094 - DNA, 39 bp, Artificial Sequence
  Description of Artificial Sequence Synthetic probe
cagggcaggtatacgccttgtttttgaagttaccgtttt

SEQ ID NO: 1095 - DNA, 39 bp, Artificial Sequence
  Description of Artificial Sequence Synthetic probe
cacttccggctccaaacaaatttttctgagtcaaagcat

SEQ ID NO: 1096 - DNA, 38 bp, Artificial Sequence
  Description of Artificial Sequence Synthetic probe
tgtcaacagcctccgcaaatttttgaagttaccgtttt

SEQ ID NO: 1097 - DNA, 40 bp, Artificial Sequence
  Description of Artificial Sequence Synthetic probe
gggcactgtagctgaagatgctttttctgagtcaaagcat

SEQ ID NO: 1098 - DNA, 37 bp, Artificial Sequence
  Description of Artificial Sequence Synthetic probe
gcagaaaggcattcccgctttttgaagttaccgtttt

SEQ ID NO: 1099 - DNA, 38 bp, Artificial Sequence
  Description of Artificial Sequence Synthetic probe
tgcacaggtgtccggctcttttttctgagtcaaagcat

SEQ ID NO: 1100 - DNA, 38 bp, Artificial Sequence
  Description of Artificial Sequence Synthetic probe
ggtccaggtacggagcgagtttttgaagttaccgtttt

SEQ ID NO: 1101 - DNA, 40 bp, Artificial Sequence
  Description of Artificial Sequence Synthetic probe
catgtgggctatgattcagcatttttctgagtcaaagcat

SEQ ID NO: 1102 - DNA, 40 bp, Artificial Sequence
  Description of Artificial Sequence Synthetic probe
taaacgctgcttttacctggatttttgaagttaccgtttt

SEQ ID NO: 1103 - DNA, 46 bp, Artificial Sequence
  Description of Artificial Sequence Synthetic probe
taatttcgtaagaatgagtttcttcattttttctgagtcaaagcat

SEQ ID NO: 1104 - DNA, 23 bp, Artificial Sequence
  Description of Artificial Sequence Synthetic probe
taactgggatggatattgacagg

SEQ ID NO: 1105 - DNA, 23 bp, Artificial Sequence
  Description of Artificial Sequence Synthetic probe
tgattttcaccgcaagacatgta

SEQ ID NO: 1106 - DNA, 20 bp, Artificial Sequence
  Description of Artificial Sequence Synthetic probe
ggtttgggggtctgacacca

SEQ ID NO: 1107 - DNA, 20 bp, Artificial Sequence
  Description of Artificial Sequence Synthetic probe
ggccagctgggacttaagct

SEQ ID NO: 1108 - DNA, 39 bp, Artificial Sequence
  Description of Artificial Sequence Synthetic probe
gcctgtgctgctcgaggatttttctcttggaaagaaagt

SEQ ID NO: 1109 - DNA, 43 bp, Artificial Sequence
  Description of Artificial Sequence Synthetic probe
tgaagaagaagaccgcttgctttttttctcttggaaagaaagt

SEQ ID NO: 1110 - DNA, 40 bp, Artificial Sequence
  Description of Artificial Sequence Synthetic probe
catgaggacctcgattgcgtttttctcttggaaagaaagt

SEQ ID NO: 1111 - DNA, 37 bp, Artificial Sequence
  Description of Artificial Sequence Synthetic probe
cgcggggatgatgggctttttctcttggaaagaaagt

SEQ ID NO: 1112 - DNA, 40 bp, Artificial Sequence
  Description of Artificial Sequence Synthetic probe
gcttggggaagaaggtgcatttttctcttggaaagaaagt

SEQ ID NO: 1113 - DNA, 41 bp, Artificial Sequence
  Description of Artificial Sequence Synthetic probe
ccttccagaacgcctcatgatttttctcttggaaagaaagt

SEQ ID NO: 1114 - DNA, 43 bp, Artificial Sequence
  Description of Artificial Sequence Synthetic probe
tccttcagcttggttttgaggttttttctcttggaaagaaagt

SEQ ID NO: 1115 - DNA, 42 bp, Artificial Sequence
  Description of Artificial Sequence Synthetic probe
gagtgccaactgggcctctactttttctcttggaaagaaagt

SEQ ID NO: 1116 - DNA, 41 bp, Artificial Sequence
  Description of Artificial Sequence Synthetic probe
tttcacaaggcctgactttgattttttgaagttaccgtttt

SEQ ID NO: 1117 - DNA, 43 bp, Artificial Sequence
  Description of Artificial Sequence Synthetic probe
gctaaaatcagcttcactacctggtttttctgagtcaaagcat

SEQ ID NO: 1118 - DNA, 38 bp, Artificial Sequence
  Description of Artificial Sequence Synthetic probe
cctcaggagcggaaatgcttttttgaagttaccgtttt

SEQ ID NO: 1119 - DNA, 38 bp, Artificial Sequence
  Description of Artificial Sequence Synthetic probe
cccgcagtcgacatatggatttttctgagtcaaagcat

SEQ ID NO: 1120 - DNA, 39 bp, Artificial Sequence
  Description of Artificial Sequence Synthetic probe
cgaagctccattactccgcctttttgaagttaccgtttt

SEQ ID NO: 1121 - DNA, 38 bp, Artificial Sequence
  Description of Artificial Sequence Synthetic probe
tgctgccaggcttgtgtgatttttctgagtcaaagcat

SEQ ID NO: 1122 - DNA, 41 bp, Artificial Sequence
  Description of Artificial Sequence Synthetic probe
catggacagaaggtccttttcctttttgaagttaccgtttt

SEQ ID NO: 1123 - DNA, 40 bp, Artificial Sequence
  Description of Artificial Sequence Synthetic probe
gctcagctgattcctgcacagtttttctgagtcaaagcat

SEQ ID NO: 1124 - DNA, 43 bp, Artificial Sequence
  Description of Artificial Sequence Synthetic probe
tttgtaggagcctgagttgctacttttttgaagttaccgtttt

SEQ ID NO: 1125 - DNA, 38 bp, Artificial Sequence
  Description of Artificial Sequence Synthetic probe
tggggctacagtcgcttcctttttctgagtcaaagcat

SEQ ID NO: 1126 - DNA, 43 bp, Artificial Sequence
  Description of Artificial Sequence Synthetic probe
gcagtggagtcaaatactgctctgtttttgaagttaccgtttt

SEQ ID NO: 1127 - DNA, 43 bp, Artificial Sequence
  Description of Artificial Sequence Synthetic probe
gtctcactgtcacctctttctgcttttttctgagtcaaagcat

SEQ ID NO: 1128 - DNA, 38 bp, Artificial Sequence
  Description of Artificial Sequence Synthetic probe
atggagtcggcgctcagattttttgaagttaccgtttt

SEQ ID NO: 1129 - DNA, 41 bp, Artificial Sequence
  Description of Artificial Sequence Synthetic probe
ccacgatttcactttccctttctttttctgagtcaaagcat

SEQ ID NO: 1130 - DNA, 37 bp, Artificial Sequence
  Description of Artificial Sequence Synthetic probe
ccagtcctctcgcatgcgtttttgaagttaccgtttt

SEQ ID NO: 1131 - DNA, 40 bp, Artificial Sequence
  Description of Artificial Sequence Synthetic probe
ccggtgacactcctcctcaattttttctgagtcaaagcat

SEQ ID NO: 1132 - DNA, 16 bp, Artificial Sequence
  Description of Artificial Sequence Synthetic probe
tggagcttgggggccg

SEQ ID NO: 1133 - DNA, 14 bp, Artificial Sequence
  Description of Artificial Sequence Synthetic probe
gcccgggccccctc

SEQ ID NO: 1134 - DNA, 13 bp, Artificial Sequence
  Description of Artificial Sequence Synthetic probe
cccggcgccggcc

SEQ ID NO: 1135 - DNA, 20 bp, Artificial Sequence
  Description of Artificial Sequence Synthetic probe
catgcttcagagccccacac

SEQ ID NO: 1136 - DNA, 21 bp, Artificial Sequence
  Description of Artificial Sequence Synthetic probe
ccacttgtacaacaccccctc

SEQ ID NO: 1137 - DNA, 22 bp, Artificial Sequence
  Description of Artificial Sequence Synthetic probe
gccatctttatgcttcctttgc

SEQ ID NO: 1138 - DNA, 25 bp, Artificial Sequence
  Description of Artificial Sequence Synthetic probe
agtccttgtatcagtcaaacatgct

SEQ ID NO: 1139 - DNA, 27 bp, Artificial Sequence
  Description of Artificial Sequence Synthetic probe
ggtttcacttatttctttttctttttt

SEQ ID NO: 1140 - DNA, 35 bp, Artificial Sequence
  Description of Artificial Sequence Synthetic probe
cagcgggcgcagcgtttttctcttggaaagaaagt

SEQ ID NO: 1141 - DNA, 39 bp, Artificial Sequence
  Description of Artificial Sequence Synthetic probe
ggcctttagaggccgggatttttctcttggaaagaaagt

SEQ ID NO: 1142 - DNA, 36 bp, Artificial Sequence
  Description of Artificial Sequence Synthetic probe
cgccggcacgaagcctttttctcttggaaagaaagt

SEQ ID NO: 1143 - DNA, 35 bp, Artificial Sequence
  Description of Artificial Sequence Synthetic probe
gcccctcccggccctttttctcttggaaagaaagt

SEQ ID NO: 1144 - DNA, 36 bp, Artificial Sequence
  Description of Artificial Sequence Synthetic probe
cgcgcatcctcccggtttttctcttggaaagaaagt

SEQ ID NO: 1145 - DNA, 48 bp, Artificial Sequence
  Description of Artificial Sequence Synthetic probe
gctgaatgaactttaatttcacaaacttttttctcttggaaagaaagt

SEQ ID NO: 1146 - DNA, 43 bp, Artificial Sequence
  Description of Artificial Sequence Synthetic probe
gctgcattcactgccttcatgttttttctcttggaaagaaagt

SEQ ID NO: 1147 - DNA, 43 bp, Artificial Sequence
  Description of Artificial Sequence Synthetic probe
ttttggttttcagcgattcacttttttctcttggaaagaaagt

SEQ ID NO: 1148 - DNA, 39 bp, Artificial Sequence
  Description of Artificial Sequence Synthetic probe
agacattcccgggtaagccttttttgaagttaccgtttt

SEQ ID NO: 1149 - DNA, 33 bp, Artificial Sequence
  Description of Artificial Sequence Synthetic probe
cgaggcgcgggccctttttctgagtcaaagcat

SEQ ID NO: 1150 - DNA, 35 bp, Artificial Sequence
  Description of Artificial Sequence Synthetic probe
gccgcagggacgtggctttttgaagttaccgtttt

SEQ ID NO: 1151 - DNA, 36 bp, Artificial Sequence
  Description of Artificial Sequence Synthetic probe
gctttctgcctgcgcgctttttctgagtcaaagcat

SEQ ID NO: 1152 - DNA, 34 bp, Artificial Sequence
  Description of Artificial Sequence Synthetic probe
gcagggcctggggcatttttgaagttaccgtttt

SEQ ID NO: 1153 - DNA, 36 bp, Artificial Sequence
  Description of Artificial Sequence Synthetic probe
gcggtgatggggtaggctttttctgagtcaaagcat

SEQ ID NO: 1154 - DNA, 41 bp, Artificial Sequence
  Description of Artificial Sequence Synthetic probe
gccagcctgtgagatagttggttttttgaagttaccgtttt

SEQ ID NO: 1155 - DNA, 41 bp, Artificial Sequence
  Description of Artificial Sequence Synthetic probe
tctaaaacaaaccaacgaggcttttttctgagtcaaagcat

SEQ ID NO: 1156 - DNA, 47 bp, Artificial Sequence
  Description of Artificial Sequence Synthetic probe
tgaatcatagtaggataagattccattatttttgaagttaccgtttt

SEQ ID NO: 1157 - DNA, 41 bp, Artificial Sequence
  Description of Artificial Sequence Synthetic probe
tccctttgcaaacatcatcttgtttttctgagtcaaagcat

SEQ ID NO: 1158 - DNA, 45 bp, Artificial Sequence
  Description of Artificial Sequence Synthetic probe
tgattaattccattcttgtgttgtcttttttgaagttaccgtttt

SEQ ID NO: 1159 - DNA, 40 bp, Artificial Sequence
  Description of Artificial Sequence Synthetic probe
agaaatgctgctctccaggaatttttctgagtcaaagcat

SEQ ID NO: 1160 - DNA, 40 bp, Artificial Sequence
  Description of Artificial Sequence Synthetic probe
accagccacctctgtctttcatttttgaagttaccgtttt

SEQ ID NO: 1161 - DNA, 37 bp, Artificial Sequence
  Description of Artificial Sequence Synthetic probe
ttggagctccccagagcgtttttctgagtcaaagcat

SEQ ID NO: 1162 - DNA, 43 bp, Artificial Sequence
  Description of Artificial Sequence Synthetic probe
cacagtagaggcgaagttcagacatttttgaagttaccgtttt

SEQ ID NO: 1163 - DNA, 43 bp, Artificial Sequence
  Description of Artificial Sequence Synthetic probe
tatgaacttgctgcattaagaggttttttctgagtcaaagcat

SEQ ID NO: 1164 - DNA, 29 bp, Artificial Sequence
  Description of Artificial Sequence Synthetic probe
cattcaaatttcttatgactgataattta

SEQ ID NO: 1165 - DNA, 24 bp, Artificial Sequence
  Description of Artificial Sequence Synthetic probe
cttggtcaatgaagagaacttggt

SEQ ID NO: 1166 - DNA, 42 bp, Artificial Sequence; identical to SEQ ID NO: 786
  Description of Artificial Sequence Synthetic probe

SEQ ID NO: 1167 - DNA, 41 bp, Artificial Sequence; identical to SEQ ID NO: 787
  Description of Artificial Sequence Synthetic probe

SEQ ID NO: 1168 - DNA, 44 bp, Artificial Sequence; identical to SEQ ID NO: 789
  Description of Artificial Sequence Synthetic probe

SEQ ID NO: 1169 - DNA, 44 bp, Artificial Sequence
  Description of Artificial Sequence Synthetic probe
atcttcaaatagaggccgatttctttttctcttggaaagaaagt

SEQ ID NO: 1170 - DNA, 48 bp, Artificial Sequence
  Description of Artificial Sequence Synthetic probe
cctctaggctggctatctttatacatatttttctcttggaaagaaagt

SEQ ID NO: 1171 - DNA, 48 bp, Artificial Sequence
  Description of Artificial Sequence Synthetic probe
cacttcacagagatagttacagccatatttttctcttggaaagaaagt

SEQ ID NO: 1172 - DNA, 40 bp, Artificial Sequence
  Description of Artificial Sequence Synthetic probe
tgcactgggagacaattcctttttttgaagttaccgtttt

SEQ ID NO: 1173 - DNA, 37 bp, Artificial Sequence
  Description of Artificial Sequence Synthetic probe
gcagccaggagggcaaaatttttctgagtcaaagcat

SEQ ID NO: 1174 - DNA, 39 bp, Artificial Sequence
  Description of Artificial Sequence Synthetic probe
ttcctcttcccgaagctgtgtttttgaagttaccgtttt

SEQ ID NO: 1175 - DNA, 42 bp, Artificial Sequence
  Description of Artificial Sequence Synthetic probe
gatctggaaggtctgaggttccttttttctgagtcaaagcat

SEQ ID NO: 1176 - DNA, 40 bp, Artificial Sequence
  Description of Artificial Sequence Synthetic probe
gccatctttattcctgcgacatttttgaagttaccgtttt

SEQ ID NO: 1177 - DNA, 43 bp, Artificial Sequence
  Description of Artificial Sequence Synthetic probe
caattgtcttctactggttcagcatttttctgagtcaaagcat

SEQ ID NO: 1178 - DNA, 42 bp, Artificial Sequence
  Description of Artificial Sequence Synthetic probe
atttcattgccacaaagttgatgtttttgaagttaccgtttt

SEQ ID NO: 1179 - DNA, 47 bp, Artificial Sequence
  Description of Artificial Sequence Synthetic probe
gctataaagtaaagcgtattgtcaataatttttctgagtcaaagcat

SEQ ID NO: 1180 - DNA, 43 bp, Artificial Sequence
  Description of Artificial Sequence Synthetic probe
gattccaggttttcatcatcttcatttttgaagttaccgtttt

SEQ ID NO: 1181 - DNA, 43 bp, Artificial Sequence
  Description of Artificial Sequence Synthetic probe
gattcaagcttgccaaagtaatcttttttctgagtcaaagcat

SEQ ID NO: 1182 - DNA, 48 bp, Artificial Sequence
  Description of Artificial Sequence Synthetic probe
gcattatctctacagtcagaatcagtcattttttgaagttaccgtttt

SEQ ID NO: 1183 - DNA, 43 bp, Artificial Sequence
  Description of Artificial Sequence Synthetic probe
cttataataaatatggtccggggttttttctgagtcaaagcat

SEQ ID NO: 1184 - DNA, 23 bp, Artificial Sequence
  Description of Artificial Sequence Synthetic probe
ggcaacaatatccactttaccag

SEQ ID NO: 1185 - DNA, 20 bp, Artificial Sequence
  Description of Artificial Sequence Synthetic probe
gacgtactcagcgccagcat

SEQ ID NO: 1186 - DNA, 39 bp, Artificial Sequence
  Description of Artificial Sequence Synthetic probe
gaggctgcgggctcaatttttttctcttggaaagaaagt

SEQ ID NO: 1187 - DNA, 41 bp, Artificial Sequence
  Description of Artificial Sequence Synthetic probe
tggcgacgcaaaagaagatgtttttctcttggaaagaaagt

SEQ ID NO: 1188 - DNA, 42 bp, Artificial Sequence
  Description of Artificial Sequence Synthetic probe
caaatccgttgactccgaccttttttctcttggaaagaaagt

SEQ ID NO: 1189 - DNA, 43 bp, Artificial Sequence
  Description of Artificial Sequence Synthetic probe
ggtcaatgaaggggtcattgattttttctcttggaaagaaagt

SEQ ID NO: 1190 - DNA, 41 bp, Artificial Sequence
  Description of Artificial Sequence Synthetic probe
ccttgacggtgccatggaattttttctcttggaaagaaagt

SEQ ID NO: 1191 - DNA, 41 bp, Artificial Sequence
  Description of Artificial Sequence Synthetic probe
aagacgccagtggactccactttttctcttggaaagaaagt

SEQ ID NO: 1192 - DNA, 38 bp, Artificial Sequence
  Description of Artificial Sequence Synthetic probe
ggagcagagagcgaagcggtttttgaagttaccgtttt

SEQ ID NO: 1193 - DNA, 39 bp, Artificial Sequence
  Description of Artificial Sequence Synthetic probe
cggctgactgtcgaacaggatttttctgagtcaaagcat

SEQ ID NO: 1194 - DNA, 36 bp, Artificial Sequence
  Description of Artificial Sequence Synthetic probe
gagcgatgtggctcggctttttgaagttaccgtttt

SEQ ID NO: 1195 - DNA, 39 bp, Artificial Sequence
  Description of Artificial Sequence Synthetic probe
tcaccttccccatggtgtcttttttctgagtcaaagcat

SEQ ID NO: 1196 - DNA, 37 bp, Artificial Sequence
  Description of Artificial Sequence Synthetic probe
ccaggcgcccaatacgactttttgaagttaccgtttt

SEQ ID NO: 1197 - DNA, 40 bp, Artificial Sequence
  Description of Artificial Sequence Synthetic probe
agttaaaagcagccctggtgatttttctgagtcaaagcat

SEQ ID NO: 1198 - DNA, 44 bp, Artificial Sequence
  Description of Artificial Sequence Synthetic probe
tggaacatgtaaaccatgtagttgatttttgaagttaccgtttt

SEQ ID NO: 1199 - DNA, 40 bp, Artificial Sequence
  Description of Artificial Sequence Synthetic probe
ttgccatgggtggaatcatattttttctgagtcaaagcat

SEQ ID NO: 1200 - DNA, 40 bp, Artificial Sequence
  Description of Artificial Sequence Synthetic probe
tgacaagcttcccgttctcagtttttgaagttaccgtttt

SEQ ID NO: 1201 - DNA, 40 bp, Artificial Sequence
  Description of Artificial Sequence Synthetic probe
tggtgatgggatttccattgatttttctgagtcaaagcat

SEQ ID NO: 1202 - DNA, 39 bp, Artificial Sequence
  Description of Artificial Sequence Synthetic probe
gggatctcgctcctggaagatttttgaagttaccgtttt

SEQ ID NO: 1203 - DNA, 38 bp, Artificial Sequence
  Description of Artificial Sequence Synthetic probe
cgccccacttgattttggatttttctgagtcaaagcat

SEQ ID NO: 1204 - DNA, 19 bp, Artificial Sequence
  Description of Artificial Sequence Synthetic oligonucleotide
tgaccaaggaaatcggcct

SEQ ID NO: 1205 - DNA, 19 bp, Artificial Sequence
  Description of Artificial Sequence Synthetic oligonucleotide
tcaccaagggcatcggcca

SEQ ID NO: 1206 - DNA, 19 bp, Artificial Sequence
  Description of Artificial Sequence Synthetic oligonucleotide
tgcccgaggaagtcggcct

SEQ ID NO: 1207 - DNA, 19 bp, Artificial Sequence
  Description of Artificial Sequence Synthetic oligonucleotide
agaccaagaacctcggcct

SEQ ID NO: 1208 - DNA, 19 bp, Artificial Sequence
  Description of Artificial Sequence Synthetic oligonucleotide
agaccaagggcctcggccg

SEQ ID NO: 1209 - DNA, 19 bp, Artificial Sequence
  Description of Artificial Sequence Synthetic oligonucleotide
agacaaaggaaatccgccg

SEQ ID NO: 1210 - DNA, 19 bp, Artificial Sequence
  Description of Artificial Sequence Synthetic oligonucleotide
agaccaaggacatgggcct

SEQ ID NO: 1211 - DNA, 19 bp, Artificial Sequence
  Description of Artificial Sequence Synthetic oligonucleotide
agaggaaggaaatcagcct

SEQ ID NO: 1212 - DNA, 19 bp, Artificial Sequence
  Description of Artificial Sequence Synthetic oligonucleotide
agacccagggaatgggccc

SEQ ID NO: 1213 - DNA, 19 bp, Artificial Sequence
  Description of Artificial Sequence Synthetic oligonucleotide
tgagcaagtaaattggcct

SEQ ID NO: 1214 - DNA, 19 bp, Artificial Sequence
  Description of Artificial Sequence Synthetic oligonucleotide
acaccaaggatatcggcac

SEQ ID NO: 1215 - DNA, 19 bp, Artificial Sequence
  Description of Artificial Sequence Synthetic oligonucleotide
ctaccaaggatatcagcct

SEQ ID NO: 1216 - DNA, 19 bp, Artificial Sequence
  Description of Artificial Sequence Synthetic oligonucleotide
agaccaaggaaatggcccg

SEQ ID NO: 1217 - DNA, 19 bp, Artificial Sequence
  Description of Artificial Sequence Synthetic oligonucleotide
aggccgacttccttggtgg

SEQ ID NO: 1218 - DNA, 19 bp, Artificial Sequence
  Description of Artificial Sequence Synthetic oligonucleotide
aggcctatttccttggtag

SEQ ID NO: 1219 - DNA, 19 bp, Artificial Sequence
  Description of Artificial Sequence Synthetic oligonucleotide
actctctcctgtgagaaca

SEQ ID NO: 1220 - DNA, 19 bp, Artificial Sequence
  Description of Artificial Sequence Synthetic oligonucleotide
tctggctactgtgagaaca

SEQ ID NO: 1221 - DNA, 19 bp, Artificial Sequence
  Description of Artificial Sequence Synthetic oligonucleotide
actcccacttgtgagaaca

SEQ ID NO: 1222 - DNA, 19 bp, Artificial Sequence
  Description of Artificial Sequence Synthetic oligonucleotide
cctttcttctgggagaact

SEQ ID NO: 1223 - DNA, 19 bp, Artificial Sequence
  Description of Artificial Sequence Synthetic oligonucleotide
actctatcctgtgagacca

SEQ ID NO: 1224 - DNA, 19 bp, Artificial Sequence
  Description of Artificial Sequence Synthetic oligonucleotide
actgtctcctgtgaaaaca

SEQ ID NO: 1225 - DNA, 19 bp, Artificial Sequence
  Description of Artificial Sequence Synthetic oligonucleotide
cctctctactgtgagaaat

SEQ ID NO: 1226 - DNA, 19 bp, Artificial Sequence
  Description of Artificial Sequence Synthetic oligonucleotide
cctgtctcctgtgagaaag

SEQ ID NO: 1227 - DNA, 19 bp, Artificial Sequence
  Description of Artificial Sequence Synthetic oligonucleotide
cctttctcctgtgtgaact

SEQ ID NO: 1228 - DNA, 19 bp, Artificial Sequence
  Description of Artificial Sequence Synthetic oligonucleotide
tctcgctcctgttagaaca

SEQ ID NO: 1229 - DNA, 19 bp, Artificial Sequence
  Description of Artificial Sequence Synthetic oligonucleotide
tctctctgctgtgaaaact

SEQ ID NO: 1230 - DNA, 19 bp, Artificial Sequence
  Description of Artificial Sequence Synthetic oligonucleotide
tctctgtcctgtgagaagc

SEQ ID NO: 1231 - DNA, 19 bp, Artificial Sequence
  Description of Artificial Sequence Synthetic oligonucleotide
cctctctccagtgagaatc

SEQ ID NO: 1232 - DNA, 19 bp, Artificial Sequence
  Description of Artificial Sequence Synthetic oligonucleotide
ggttctcacaggacagagc

SEQ ID NO: 1233 - DNA, 19 bp, Artificial Sequence
  Description of Artificial Sequence Synthetic oligonucleotide
agttctgagaggagagagg

SEQ ID NO: 1234 - DNA, 19 bp, Artificial Sequence
  Description of Artificial Sequence Synthetic oligonucleotide
tgttcactgttcaaaacga

SEQ ID NO: 1235 - DNA, 19 bp, Artificial Sequence
  Description of Artificial Sequence Synthetic oligonucleotide
tgatctgtgttcaaaacgt

SEQ ID NO: 1236 - DNA, 19 bp, Artificial Sequence
  Description of Artificial Sequence Synthetic oligonucleotide
ttttcacctttcaaaacgg

SEQ ID NO: 1237 - DNA, 19 bp, Artificial Sequence
  Description of Artificial Sequence Synthetic oligonucleotide
atttcattgtttaaaacgt

SEQ ID NO: 1238 - DNA, 19 bp, Artificial Sequence
  Description of Artificial Sequence Synthetic oligonucleotide
cgttcattatttaaaacgt

SEQ ID NO: 1239 - DNA, 19 bp, Artificial Sequence
  Description of Artificial Sequence Synthetic oligonucleotide
ggttcacccttgaaaacgt

SEQ ID NO: 1240 - DNA, 19 bp, Artificial Sequence
  Description of Artificial Sequence Synthetic oligonucleotide
agctcactgttcaaaaagt

SEQ ID NO: 1241 - DNA, 19 bp, Artificial Sequence
  Description of Artificial Sequence Synthetic oligonucleotide
tgttcactgtttaaaaccg

SEQ ID NO: 1242 - DNA, 19 bp, Artificial Sequence
  Description of Artificial Sequence Synthetic oligonucleotide
cattcattgttcaaaaggt

SEQ ID NO: 1243 - DNA, 19 bp, Artificial Sequence
  Description of Artificial Sequence Synthetic oligonucleotide
taatcactgttcaaaagga

SEQ ID NO: 1244 - DNA, 19 bp, Artificial Sequence
  Description of Artificial Sequence Synthetic oligonucleotide
tgctcagtgttcaaaacca

SEQ ID NO: 1245 - DNA, 19 bp, Artificial Sequence
  Description of Artificial Sequence Synthetic oligonucleotide
tgttcacagtttaaaacat

SEQ ID NO: 1246 - DNA, 19 bp, Artificial Sequence
  Description of Artificial Sequence Synthetic oligonucleotide
tgttcactgttcaaaaaat

SEQ ID NO: 1247 - DNA, 19 bp, Artificial Sequence
  Description of Artificial Sequence Synthetic oligonucleotide
aagtttataacagtgaact

SEQ ID NO: 1248 - DNA, 19 bp, Artificial Sequence
  Description of Artificial Sequence Synthetic oligonucleotide
ttattttgaaaagtgaaca

(19) United States
(12) Patent Application Publication
(10) Pub. No.: US 2011/0152349 A1
(43) Pub. Date: Jun. 23, 2011

(54) COMPOSITIONS AND METHODS FOR INHIBITING EXPRESSION OF IL-18 GENES

(76) Inventors: Anke Geick, Bayreuth (DE); Markus Hossbach, Kulmbach (DE); Grace Ju, Montclair, NJ (US); Ingo Roehl, Memmelsdorf (DE)

(21) Appl. No.: 12/967,432

(22) Filed: Dec. 14, 2010

(30) Foreign Application Priority Data
Dec. 18, 2009 (EP) 09179873.6

(51) Int. Cl. A61K 31/713; C07H 21/02; C12N 15/63; C12N 5/10; A61P 37/02

(52) U.S. Cl. 514/44 A; 536/23.1; 435/320.1; 435/325

(57) ABSTRACT

The invention relates to a double-stranded ribonucleic acid (dsRNA) for inhibiting the expression of a IL-18 gene. The invention also relates to a pharmaceutical composition comprising the dsRNA or nucleic acid molecules or vectors encoding the same together with a pharmaceutically acceptable carrier; methods for treating diseases caused by the expression of a IL-18 gene using said pharmaceutical composition; and methods for inhibiting the expression of IL-18 in a cell.

### PRIORITY TO RELATED APPLICATION(S)

This application claims the benefit of European Patent Application No. 09179873.6, filed Dec. 18, 2009, which is hereby incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Nov. 10, 2010, is named 26499.txt and is 351,354 bytes in size.

### BACKGROUND OF THE INVENTION

IL-18 is a proinflammatory cytokine which belongs to the IL-1 superfamily and is mainly produced by macrophages and dendritic cells, but also other cell types, as e.g. synovial fibroblasts, keratinocytes, and osteoblasts, and airway epithelial cells. IL-18 is produced intracellularly from a biologically inactivated precursor, pro-IL-18. Mature IL-18 is secreted after cleavage of pro-IL-18 by caspase-1, originally identified as IL-1β converting enzyme. Activated macrophages produce large amounts of mature IL-18 after cleavage of pro-IL-18 by caspase-1.

IL-18 was first identified as an inducer of interferon gamma (IFN-γ). It acts synergistically with IL-12 to induce IFN-γ from several cell types. IL-18 plays a role in both T cell and NK cell maturation, and has been implicated in Th17 cell responses. IL-18 can induce TNF-α, IL-1β, chemokines such as IL-8, CXCL5, and CCL20, and other inflammatory mediators associated with the pathogenesis of several autoimmune diseases. In addition, IL-18 enhances the production of the angiogenic factor VEGF in rheumatoid synovial tissue. Thus, the biological effects of IL-18 on a variety of cell types has implicated this cytokine in the pathology of several inflammatory diseases.

The role of IL-18 in respiratory disorders has been highlighted by numerous studies in mice and in humans. It has been demonstrated that IL-18 is induced in lungs of mice and humans exposed to cigarette smoke and that its expression level is elevated in COPD lungs and sera as well as sputum macrophages. Transgenic mice expressing IL-18 specifically in airway type II-cells under the control of the SP-C promoter develop lung fibrosis and emphysema and a knock-out mouse deficient for the Interleukin-18 receptor shows a strongly reduced production of chemokines, inflammation and emphysema in response to cigarette smoke as compared to normal mice.

IL-18 can act as a co-factor for both Th1 and Th2 cell development, and several studies have subsequently reported that IL-18 may be involved in the development of Th2-type-diseases, such as asthma. In an ovalbumin-induced murine model of asthma, increased expression of IL-18 was observed in lung macrophages and bronchial epithelial cells, and treatment which resulted in a reduction in IL-18 expression correlated with decreased airway inflammation and bronchial hyperresponsiveness. Enhanced expression of IL-18 has been observed in patients with bleomycin-induced lung injury as well as in the lungs of mice treated with bleomycin. These results show IL-18 is involved in the pathogenesis of pulmonary inflammatory diseases, such as lung fibrosis, lung injury, asthma and COPD.

IL-18 also plays a role in autoimmune diseases. IL-18 is expressed in synovial tissue and sera of rheumatoid arthritis patients. Administration of IL-18 to mice with collagen-induced arthritis (CIA) significantly enhanced the development and severity of disease. Patients with Crohn's disease have elevated levels of circulating IL-18. IL-18-deficient mice have been shown to be resistant to experimental colitis, in contrast to IL-18 transgenic mice which develop intestinal inflammation.

IL-18 has been implicated in other diseases associated with or exacerbated by an inflammatory component. IL-18 is overexpressed in macrophages found in artherosclerotic plaques in patients. Higher levels of IL-18 were associated with symptomatic (unstable) plaques, suggesting a role for IL-18 in plaque destabilization. In a rodent model of artherosclerosis, inhibition of IL-18 by IL-18 binding protein attenuated the development of atherosclerotic lesions. Elevated levels of IL-18 are found in various tumors.

Data from patients and from rodent models clearly indicate that IL-18 is an important mediator of the inflammatory response in respiratory diseases like COPD and asthma, in acute lung injury, and in autoimmune diseases such as rheumatoid arthritis and inflammatory bowel disease. IL-18 may also contribute to the pathology of atherosclerosis. These afore-named diseases represent diseases of significant unmet medical need. COPD is responsible for about 100,000 cases of death per year in the US with increasing prevalence. Statistical extrapolations predict that COPD will be the third leading cause of death worldwide by 2020. Current therapies do not treat underlying inflammation and tissue damage, which is considered to be steroid resistant. About 46 million patients worldwide suffer from asthma. The symptoms of the disease are transiently reversible by treatment with inhaled glucocorticoids. Treatment of severe, steroid resistant asthma and exacerbations is still an unmet medical need. Despite medical advances in the treatment of atherosclerosis, it remains one of the leading causes of mortality and morbidity in the world.

Double-stranded RNA molecules (dsRNA) have been shown to block gene expression in a highly conserved regulatory mechanism known as RNA interference (RNAi). Downregulation of IL-18 by an IL-18 specific siRNA is expected to reduce the inflammatory effects of IL-18 and thus ameliorate diseases in which this proinflammatory cytokine plays an important role.

### SUMMARY OF THE INVENTION

The present invention relates to double-stranded ribonucleic acid molecules (dsRNAs), as well as compositions and methods for inhibiting the expression of the IL-18 gene, in particular the expression of the IL-18 gene, in a cell, tissue or mammal using such dsRNA. The invention also provides compositions and methods for treating pathological conditions and diseases caused by the expression of the IL-18 gene such as autoimmune and inflammatory diseases like atherosclerosis, rheumatoid arthritis and inflammatory bowel disease, as well as respiratory diseases/disorders like asthma and COPD, acute lung injury, and pulmonary fibrosis.

In one preferred embodiment the described dsRNA molecule is capable of inhibiting the expression of a IL-18 gene by at least 60%, preferably by at least 70%, most preferably by at least 80%. The invention also provides compositions and methods for specifically targeting the lung with IL-18 dsRNA, for treating pathological conditions and diseases caused by the expression of the IL-18 gene including those described above.

### DETAILED DESCRIPTION OF THE INVENTION

The invention provides double-stranded ribonucleic acid (dsRNA) molecules able to selectively and efficiently decrease the expression of IL-18. The use of IL-18 RNAi provides a method for the therapeutic and/or prophylactic treatment of diseases/disorders which are associated with autoimmune and inflammatory diseases. Such diseases/disorders include atherosclerosis, rheumatoid arthritis and inflammatory bowel disease, as well as respiratory diseases/disorders like asthma and COPD, acute lung injury, and pulmonary fibrosis, which method comprises administration of dsRNA targeting IL-18 to a human being or animal.

In one embodiment, the invention provides double-stranded ribonucleic acid (dsRNA) molecules for inhibiting the expression of a IL-18 gene, in particular the expression of the mammalian or human IL-18 gene. The dsRNA comprises at least two sequences that are complementary to each other. The dsRNA comprises a sense strand comprising a first sequence and an antisense strand may comprise a second sequence, see sequences provided in the sequence listing and also provision of specific dsRNA pairs in the appended tables 1 and 2. In one embodiment the sense strand comprises a sequence which has an identity of at least 90% to at least a portion of an mRNA encoding IL-18. Said sequence is located in a region of complementarity of the sense strand to the antisense strand, preferably within nucleotides 2-7 of the 5′ terminus of the antisense strand. In one preferred embodiment the dsRNA targets particularly the human IL-18 gene. In another embodiment the dsRNA targets the mouse (Mus musculus) and rat (Rattus norvegicus) IL-18 gene.

In one embodiment, the antisense strand comprises a nucleotide sequence which is substantially complementary to at least part of an mRNA encoding said IL-18 gene, and the region of complementarity is most preferably less than 30 nucleotides in length. Furthermore, it is preferred that the length of the herein described inventive dsRNA molecules (duplex length) is in the range of about 16 to 30 nucleotides, in particular in the range of about 18 to 28 nucleotides. Particularly useful in context of this invention are duplex lengths of about 19, 20, 21, 22, 23 or 24 nucleotides. Most preferred are duplex stretches of 19, 21 or 23 nucleotides. The dsRNA, upon contacting with a cell expressing a IL-18 gene, inhibits the expression of a IL-18 gene in vitro by at least 60%, preferably by at least 70%, most preferred by 80%.

Appended Table 1 relates to preferred molecules to be used as dsRNA in accordance with this invention. Also modified dsRNA molecules are provided herein and are in particular disclosed in appended table 2, providing illustrative examples of modified dsRNA molecules of the present invention. As pointed out herein above, Table 2 provides for illustrative examples of modified dsRNAs of this invention (whereby the corresponding sense strand and antisense strand is provided in this table). The relation of the unmodified preferred molecules shown in Table 1 to the modified dsRNAs of Table 2 is illustrated in Table 12. Yet, the illustrative modifications of these constituents of the inventive dsRNAs are provided herein as examples of modifications.

Tables 3 and 4 provide for selective biological, clinically and pharmaceutical relevant parameters of certain dsRNA molecules of this invention.

Particularly useful with respect to the assessment of therapeutic dsRNAs is the set of dsRNAs targeting mouse and rat IL-18 which can be used to estimate toxicity, therapeutic efficacy, and effective dosages and in vivo half-lifes for the individual dsRNAs in an animal or cell culture model. Appended Tables 4 and 5 relate to preferred molecules targeting murine IL-18. Table 5 provides illustrative examples of modified dsRNAs targeting murine IL-18 (whereby the corresponding sense strand and antisense strand is provided in this table). Table 6 provides for selective biological, clinically and pharmaceutical relevant parameters of certain dsRNA molecules of this invention. The relation of the unmodified preferred molecules shown in Table 4 to the modified dsRNAs of Table 5 is illustrated in Table 13.

Most preferred dsRNA molecules are provided in the appended table 1 and, inter alia and preferably, wherein the sense strand is selected from the group consisting of the nucleic acid sequences depicted in SEQ ID NOs: 1, 3, 7, 9, 13, 15, 17, 19, 27 and 31 and the antisense strand is selected from the group consisting of the nucleic acid sequences depicted in SEQ ID NOs: 2, 4, 8, 10, 14, 16, 18, 20, 28 and 32. Accordingly, the inventive dsRNA molecule may, inter alia, comprise the sequence pairs selected from the group consisting of SEQ ID NOs: 1/2, 3/4, 7/8, 9/10, 13/14, 15/16, 17/18, 19/20, 27/28, and 31/32. In context of specific dsRNA molecules provided herein, pairs of SEQ ID NOs relate to corresponding sense and antisense strands sequences (5′ to 3′) as also shown in appended and included tables.

In one embodiment said dsRNA molecules comprise an antisense strand with a 3′ overhang of 1-5 nucleotides length, preferably of 1-2 nucleotides length. Preferably said overhang of the antisense strand comprises uracil or nucleotides which are complementary to the mRNA encoding IL-18.

In another preferred embodiment, said dsRNA molecules comprise a sense strand with a 3′ overhang of 1-5 nucleotides length, preferably of 1-2 nucleotides length. Preferably said overhang of the sense strand comprises uracil or nucleotides which are identical to the mRNA encoding IL-18.

In another preferred embodiment, said dsRNA molecules comprise a sense strand with a 3′ overhang of 1-5 nucleotides length, preferably of 1-2 nucleotides length, and an antisense strand with a 3′ overhang of 1-5 nucleotides length, preferably of 1-2 nucleotides length. Preferably said overhang of the sense strand comprises uracil or nucleotides which are at least 90% identical to the mRNA encoding IL-18 and said overhang of the antisense strand comprises uracil or nucleotides which are at least 90% complementary to the mRNA encoding IL-18.

The dsRNA molecules of the invention may be comprised of naturally occurring nucleotides or may be comprised of at least one modified nucleotide, such as a 2′-O-methyl modified nucleotide, a 5′ O-methyl modified nucleotide, a nucleotide comprising a 5′-phosphorothioate group, and a terminal nucleotide linked to a cholesteryl derivative or dodecanoic acid bisdecylamide group. 2′ modified nucleotides may have the additional advantage that certain immunostimulatory factors or cytokines are suppressed when the inventive dsRNA molecules are employed in vivo, for example in a medical setting.

Alternatively and non-limiting, the modified nucleotide may be chosen from the group of: a 2′-a 2′ fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. In one preferred embodiment the dsRNA molecules comprises at least one of the following modified nucleotides: a 2′-β-methyl modified nucleotide, a 5′ O-methyl modified nucleotide, a nucleotide comprising a 5′-phosphorothioate group, a 2′ fluoro modification a deoxythymidine and a 5′ phosphate group at the 5′ end of the antisense strand. Preferred dsRNA molecules comprising modified nucleotides are given in table 2.

In a preferred embodiment the inventive dsRNA molecules comprise modified nucleotides as detailed in the sequences given in table 2. In one preferred embodiment the inventive dsRNA molecule comprises sequence pairs selected from the group consisting of SEQ ID NOs: 1/2, 3/4, 7/8, 9/10, 13/14, 15/16, 17/18, 19/20, 27/28, and 31/32, and comprises overhangs at the antisense and/or sense strand of 1-2 deoxythymidines. In one preferred embodiment the inventive dsRNA molecule comprises sequence pairs selected from the group consisting of SEQ ID NOs: 1/2, 3/4, 7/8, 9/10, 13/14, 15/16, 17/18, 19/20, 27/28, and 31/32, and comprise modifications as detailed in table 2. Preferred dsRNA molecules comprising modified nucleotides are listed in table 4, with particularly preferred dsRNA molecules depicted in SEQ ID NOs: 149/150, 163/164, 165/166, 179/180, 183/184, 185/186, 187/188, 213/214, 215/216, 217/218, 239/240, 253/254, 255/256, 293/294, 435/436, 461/462, 463/464, 471/472, 475/476, 479/480 and 487/488. Most preferred dsRNA molecules are depicted in SEQ ID NOs 165/166, 239/240 and 255/256.

In another embodiment the inventive dsRNAs comprise modified nucleotides on positions different from those disclosed in tables 2. In one preferred embodiment two deoxythymidine nucleotides are found at the 3′ of both strands of the dsRNA molecule.

In one embodiment the dsRNA molecules of the invention comprise of a sense and an antisense strand wherein both strands have a half-life of at least 7 hours. In one preferred embodiment the dsRNA molecules of the invention comprise of a sense and an antisense strand wherein both strands have a half-life of at least 1.9 hours in human sputum (ARDS). In another embodiment the dsRNA molecules of the invention are non-immunostimulatory, e.g. do not stimulate IFN-alpha and TNF-alpha in vitro. In another embodiment the dsRNA molecules of the invention do stimulate IFN-alpha and TNF-alpha in vitro to a very minor degree.

The invention also provides for cells comprising at least one of the dsRNAs of the invention. The cell is preferably a mammalian cell, such as a human cell. Furthermore, also tissues and/or non-human organisms comprising the herein defined dsRNA molecules are comprised in this invention, whereby said non-human organism is particularly useful for research purposes or as a research tool, for example in drug testing.

Furthermore, the invention relates to a method for inhibiting the expression of a IL-18 gene, in particular a mammalian or human IL-18 gene, in a cell, tissue or organism comprising the following steps:

(a) introducing into the cell, tissue or organism a double-stranded ribonucleic acid (dsRNA) as defined herein;
(b) maintaining said cell, tissue or organism produced in step (a) for a time sufficient to obtain degradation of the mRNA transcript of a IL-18 gene, thereby inhibiting expression of a IL-18 gene in a given cell.

The invention also relates to pharmaceutical compositions comprising the inventive dsRNAs of this invention. These pharmaceutical compositions are particularly useful in the inhibition of the expression of a IL-18 gene in a cell, a tissue or an organism. The pharmaceutical composition comprising one or more of the dsRNA of the invention may also comprise (a) pharmaceutically acceptable carrier(s), diluent(s) and/or excipient(s).

In another embodiment, the invention provides methods for treating, preventing or managing autoimmune and inflammatory diseases like atherosclerosis, rheumatoid arthritis and inflammatory bowel disease, as well as respiratory diseases/disorders like asthma and COPD, acute lung injury, and pulmonary fibrosis, which are associated with IL-18, said method comprising administering to a subject in need of such treatment, prevention or management a therapeutically or prophylactically effective amount of one or more of the dsRNAs of the invention. Preferably, said subject is a mammal, most preferably a human patient.

In one embodiment, the invention provides a method for treating a subject having a pathological condition mediated by the expression of an IL-18 gene. Such conditions comprise disorders associated with autoimmune and inflammatory diseases like atherosclerosis, rheumatoid arthritis and inflammatory bowel disease, as well as respiratory diseases/disorders like asthma and COPD, acute lung injury, and pulmonary fibrosis.

In this embodiment, the dsRNA acts as a therapeutic agent for controlling the expression of a IL-18 gene. The method comprises administering a pharmaceutical composition of the invention to the patient (e.g., human), such that expression of a IL-18 gene is silenced. Because of their high specificity, the dsRNAs of the invention specifically target mRNAs of a IL-18 gene. In one preferred embodiment the described dsRNAs specifically decrease IL-18 mRNA levels and do not directly affect the expression and/or mRNA levels of off-target genes in the cell.

In one preferred embodiment the described dsRNA decrease IL-18 mRNA levels in the lung by at least 60%, preferably by at least 70%, most preferably by at least 80% in vivo. In another embodiment the described dsRNAs decrease IL-18 mRNA levels in vivo for at least 4 days.

In another embodiment, the invention provides vectors for inhibiting the expression of a IL-18 gene in a cell, in particular a IL-18 gene comprising a regulatory sequence operable linked to a nucleotide sequence that encodes at least one strand of one of the dsRNA of the invention.

In another embodiment, the invention provides a cell comprising a vector for inhibiting the expression of a IL-18 gene in a cell. Said vector comprises a regulatory sequence operable linked to a nucleotide sequence that encodes at least one strand of one of the dsRNA of the invention. Yet, it is preferred that said vector comprises, besides said regulatory sequence a sequence that encodes at least one “sense strand” of the inventive dsRNA and at least one “anti sense strand” of said dsRNA. It is also envisaged that the claimed cell comprises two or more vectors comprising, besides said regulatory sequences, the herein defined sequence(s) that encode(s) at least one strand of one of the dsRNA of the invention.

In one embodiment, the method comprises administering a composition comprising a dsRNA, wherein the dsRNA comprises a nucleotide sequence which is complementary to at least a part of an RNA transcript of a IL-18 gene of the mammal to be treated. As pointed out above, also vectors and cells comprising nucleic acid molecules that encode for at least one strand of the herein defined dsRNA molecules can be used as pharmaceutical compositions and may, therefore, also be employed in the herein disclosed methods of treating a subject in need of medical intervention. It is also of note that these embodiments relating to pharmaceutical compositions and to corresponding methods of treating a (human) subject also relate to approaches like gene therapy approaches. IL-18 specific dsRNA molecules as provided herein or nucleic acid molecules encoding individual strands of these inventive dsRNA molecules may also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In another aspect of the invention, IL-18 specific dsRNA molecules that modulate IL-18 gene expression activity are expressed from transcription units inserted into DNA or RNA vectors (see, e.g., Skillern, A., et al., International PCT Publication No. WO 00/22113). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant dsRNA expression vectors are preferably DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Danos and Mulligan, Proc. NatI. Acad. Sci. USA (1998) 85:6460-6464). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or U1 snRNA promoter) or preferably RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g. the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Preferably, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single IL-18 gene or multiple IL-18 genes over a period of a week or more are also contemplated by the invention. Successful introduction of the vectors of the invention into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The following detailed description discloses how to make and use the dsRNA and compositions containing dsRNA to inhibit the expression of a target IL-18 gene, as well as compositions and methods for treating diseases and disorders caused by the expression of said IL-18 gene.

### DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A”, “U” and “T” or “dT” respectively, each generally stand for a nucleotide that contains guanine, cytosine, adenine, uracil and deoxythymidine as a base, respectively. However, the term “ribonucleotide” or “nucleotide” can also refer to a modified nucleotide, as further detailed below, or a surrogate replacement moiety. Sequences comprising such replacement moieties are embodiments of the invention. As detailed below, the herein described dsRNA molecules may also comprise “overhangs”, i.e. unpaired, overhanging nucleotides which are not directly involved in the RNA double helical structure normally formed by the herein defined pair of “sense strand” and “anti sense strand”. Often, such an overhanging stretch comprises the deoxythymidine nucleotide, in most embodiments, 2 deoxythymidines in the 3′ end. Such overhangs will be described and illustrated below.

The term IL-18″ as used herein relates to interleukin-18, also known as IL18 or IL-18 and said term relates to the corresponding gene, encoded mRNA, encoded protein/polypeptide as well as functional fragments of the same. The protein encoded by this gene is a proinflammatory cytokine Preferred is the human IL-18 gene. In other preferred embodiments the dsRNAs of the invention target the IL-18 gene of human (H. sapiens) and cynomolgous monkey (Macaca fascicularis) IL-18 gene. Also dsRNAs targeting the rat (Rattus norvegicus) and mouse (Mus musculus) IL-18 gene are part of this invention. The term “IL-18 gene/sequence” does not only relate to (the) wild-type sequence(s) but also to mutations and alterations which may be comprised in said gene/sequence. Accordingly, the present invention is not limited to the specific dsRNA molecules provided herein. The invention also relates to dsRNA molecules that comprise an antisense strand that is at least 85% complementary to the corresponding nucleotide stretch of an RNA transcript of a IL-18 gene that comprises such mutations/alterations.

As used herein, “target sequence” refers to a contiguous portion of the nucleotide sequence of an mRNA molecule formed during the transcription of a IL-18 gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature. However, as detailed herein, such a “strand comprising a sequence” may also comprise modifications, like modified nucleotides.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

Sequences referred to as “fully complementary” comprise base-pairing of the oligonucleotide or polynucleotide comprising the first nucleotide sequence to the oligonucleotide or polynucleotide comprising the second nucleotide sequence over the entire length of the first and second nucleotide sequence.

However, where a first sequence is referred to as “substantially complementary” with respect to a second sequence herein, the two sequences can be fully complementary, or they may form one or more, but preferably not more than 13 mismatched base pairs upon hybridization.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

The term “double-stranded RNA”, “dsRNA molecule”, or “dsRNA”, as used herein, refers to a ribonucleic acid molecule, or complex of ribonucleic acid molecules, having a duplex structure comprising two anti-parallel and substantially complementary nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′ end of one strand and the 5′ end of the respective other strand forming the duplex structure, the connecting RNA chain is referred to as a “hairpin loop”. Where the two strands are connected covalently by means other than an uninterrupted chain of nucleotides between the 3′ end of one strand and the 5′ end of the respective other strand forming the duplex structure, the connecting structure is referred to as a “linker”. The RNA strands may have the same or a different number of nucleotides. In addition to the duplex structure, a dsRNA may comprise one or more nucleotide overhangs. The nucleotides in said “overhangs” may comprise between 0 and 5 nucleotides, whereby “0” means no additional nucleotide(s) that form(s) an “overhang” and whereas “5” means five additional nucleotides on the individual strands of the dsRNA duplex. These optional “overhangs” are located in the 3′ end of the individual strands. As will be detailed below, also dsRNA molecules which comprise only an “overhang” in one the two strands may be useful and even advantageous in context of this invention. The “overhang” comprises preferably between 0 and 2 nucleotides. Most preferably 2 “dT” (deoxythymidine) nucleotides are found at the 3′ end of both strands of the dsRNA. Also 2 “U”(uracil) nucleotides can be used as overhangs at the 3′ end of both strands of the dsRNA. Accordingly, a “nucleotide overhang” refers to the unpaired nucleotide or nucleotides that protrude from the duplex structure of a dsRNA when a 3′ end of one strand of the dsRNA extends beyond the 5′ end of the other strand, or vice versa. For example the antisense strand comprises 23 nucleotides and the sense strand comprises 21 nucleotides, forming a 2 nucleotide overhang at the 3′ end of the antisense strand. Preferably, the 2 nucleotide overhang is fully complementary to the mRNA of the target gene. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence. Where the region of complementarity is not fully complementary to the target sequence, the mismatches are most tolerated outside nucleotides 2-7 of the 5′ terminus of the antisense strand

The term “sense strand,” as used herein, refers to the strand of a dsRNA that includes a region that is substantially complementary to a region of the antisense strand. “Substantially complementary” means preferably at least 85% of the overlapping nucleotides in sense and antisense strand are complementary.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. It is, for example envisaged that the dsRNA molecules of this invention be administered to a subject in need of medical intervention. Such an administration may comprise the injection of the dsRNA, the vector or a cell of this invention into a diseased side in said subject, for example into lung tissue/cells. However, also the injection in close proximity of the diseased tissue is envisaged. In vitro introduction into a cell includes methods known in the art such as electroporation and lipofection.

The term “inflammation” as used herein refers to the biologic response of body tissue to injury, irritation, or disease which can be caused by harmful stimuli, for example, pathogens, damaged cells, or irritants. Inflammation is typically characterized by pain and swelling. Inflammation is intended to encompass both acute responses, in which inflammatory processes are active (e.g., neutrophils and leukocytes), and chronic responses, which are marked by slow progress, a shift in the type of cell present at the site of inflammation, and the formation of connective tissue.

The terms “silence”, “inhibit the expression of” and “knock down”, in as far as they refer to a IL-18 gene, herein refer to the at least partial suppression of the expression of a IL-18 gene, as manifested by a reduction of the amount of mRNA transcribed from a IL-18 gene which may be isolated from a first cell or group of cells in which a IL-18 gene is transcribed and which has or have been treated such that the expression of a IL-18 gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the IL-18 gene transcription, e.g. the amount of protein encoded by a IL-18 gene which is secreted by a cell, or the number of cells displaying a certain phenotype.

As illustrated in the appended examples and in the appended tables provided herein, the inventive dsRNA molecules are capable of inhibiting the expression of a human IL-18 by at least about 60%, preferably by at least 70%, most preferably by at least 80% in vitro assays, i.e. in vitro. The term “in vitro” as used herein includes but is not limited to cell culture assays. In another embodiment the inventive dsRNA molecules are capable of inhibiting the expression of a mouse or rat IL-18 by at least 60%. preferably by at least 70%, most preferably by at least 80%. The person skilled in the art can readily determine such an inhibition rate and related effects, in particular in light of the assays provided herein.

The term “off target” as used herein refers to all non-target mRNAs of the transcriptome that are predicted by in silico methods to hybridize to the described dsRNAs based on sequence complementarity. The dsRNAs of the present invention preferably do specifically inhibit the expression of IL-18, i.e. do not inhibit the expression of any off-target.

The term “half-life” as used herein is a measure of stability of a compound or molecule and can be assessed by methods known to a person skilled in the art, especially in light of the assays provided herein.

The term “non-immunostimulatory” as used herein refers to the absence of any induction of a immune response by the invented dsRNA molecules. Methods to determine immune responses are well know to a person skilled in the art, for example by assessing the release of cytokines, as described in the examples section.

The terms “treat”, “treatment”, and the like, mean in context of this invention to relief from or alleviation of a disorder related to IL-18 expression, like inflammation and proliferative disorders, like cancers.

As used herein, a “pharmaceutical composition” comprises a pharmacologically effective amount of a dsRNA and a pharmaceutically acceptable carrier. However, such a “pharmaceutical composition” may also comprise individual strands of such a dsRNA molecule or the herein described vector(s) comprising a regulatory sequence operably linked to a nucleotide sequence that encodes at least one strand of a sense or an antisense strand comprised in the dsRNAs of this invention. It is also envisaged that cells, tissues or isolated organs that express or comprise the herein defined dsRNAs may be used as “pharmaceutical compositions”. As used herein, “pharmacologically effective amount,” “therapeutically effective amount” or simply “effective amount” refers to that amount of an RNA effective to produce the intended pharmacological, therapeutic or preventive result.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives as known to persons skilled in the art.

It is in particular envisaged that the pharmaceutically acceptable carrier allows for the systemic administration of the dsRNAs, vectors or cells of this invention. Whereas also the enteric administration is envisaged the parenteral administration and also transdermal or transmucosal (e.g. insufflation, buccal, vaginal, anal) administration as well was inhalation of the drug are feasible ways of administering to a patient in need of medical intervention the compounds of this invention. When parenteral administration is employed, this can comprise the direct injection of the compounds of this invention into the diseased tissue or at least in close proximity. However, also intravenous, intraarterial, subcutaneous, intramuscular, intraperitoneal, intradermal, intrathecal and other administrations of the compounds of this invention are within the skill of the artisan, for example the attending physician.

For intramuscular, subcutaneous and intravenous use, the pharmaceutical compositions of the invention will generally be provided in sterile aqueous solutions or suspensions, buffered to an appropriate pH and isotonicity. In a preferred embodiment, the carrier consists exclusively of an aqueous buffer. In this context, “exclusively” means no auxiliary agents or encapsulating substances are present which might affect or mediate uptake of dsRNA in the cells that express a IL-18 gene. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate. The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in PCT publication WO 91/06309 which is incorporated by reference herein.

As used herein, a “transformed cell” is a cell into which at least one vector has been introduced from which a dsRNA molecule or at least one strand of such a dsRNA molecule may be expressed. Such a vector is preferably a vector comprising a regulatory sequence operably linked to nucleotide sequence that encodes at least one of a sense strand or an antisense strand comprised in the dsRNAs of this invention.

It can be reasonably expected that shorter dsRNAs comprising one of the sequences of Table 1 and 4 minus only a few nucleotides on one or both ends may be similarly effective as compared to the dsRNAs described above.

As pointed out above, in most embodiments of this invention, the dsRNA molecules provided herein comprise a duplex length (i.e. without “overhangs”) of about 16 to about 30 nucleotides. Particular useful dsRNA duplex lengths are about 19 to about 25 nucleotides. Most preferred are duplex structures with a length of 19 nucleotides. In the inventive dsRNA molecules, the antisense strand is at least partially complementary to the sense strand.

The dsRNA of the invention can contain one or more mismatches to the target sequence. In a preferred embodiment, the dsRNA of the invention contains no more than 13 mismatches. If the antisense strand of the dsRNA contains mismatches to a target sequence, it is preferable that the area of mismatch not be located within nucleotides 2-7 of the 5′ terminus of the antisense strand. In another embodiment it is preferable that the area of mismatch not to be located within nucleotides 2-9 of the 5′ terminus of the antisense strand.

As mentioned above, at least one end/strand of the dsRNA may have a single-stranded nucleotide overhang of 1 to 5, preferably 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Preferably, the single-stranded overhang is located at the 3′-terminal end of the antisense strand or, alternatively, at the 3′-terminal end of the sense strand. The dsRNA may also have a blunt end, preferably located at the 5′ end of the antisense strand. Preferably, the antisense strand of the dsRNA has a nucleotide overhang at the 3′ end, and the 5′ end is blunt. In another embodiment, one or more of the nucleotides in the overhang is replaced with a nucleoside thiophosphate.

The dsRNA of the present invention may also be chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, N.Y., USA, which is hereby incorporated herein by reference. Chemical modifications may include, but are not limited to 2′ modifications, introduction of non-natural bases, covalent attachment to a ligand, and replacement of phosphate linkages with thiophosphate linkages. In this embodiment, the integrity of the duplex structure is strengthened by at least one, and preferably two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues. Preferably, the chemical groups that can be used to modify the dsRNA include, without limitation, methylene blue; bifunctional groups, preferably bis-(2-chloroethyl)amine; N-acetyl-N′-(p-glyoxylbenzoyl)cystamine; 4-thiouracil; and psoralen. In one preferred embodiment, the linker is a hexa-ethylene glycol linker In this case, the dsRNA are produced by solid phase synthesis and the hexa-ethylene glycol linker is incorporated according to standard methods (e.g., Williams, D. J., and K. B. Hall, Biochem. (1996) 35:14665-14670). In a particular embodiment, the 5′ end of the antisense strand and the 3′ end of the sense strand are chemically linked via a hexaethylene glycol linker. In another embodiment, at least one nucleotide of the dsRNA comprises a phosphorothioate or phosphorodithioate groups. The chemical bond at the ends of the dsRNA is preferably formed by triple-helix bonds.

In certain embodiments, a chemical bond may be formed by means of one or several bonding groups, wherein such bonding groups are preferably poly-(oxyphosphinicooxy-1,3-propandiol)- and/or polyethylene glycol chains. In other embodiments, a chemical bond may also be formed by means of purine analogs introduced into the double-stranded structure instead of purines. In further embodiments, a chemical bond may be formed by azabenzene units introduced into the double-stranded structure. In still further embodiments, a chemical bond may be formed by branched nucleotide analogs instead of nucleotides introduced into the double-stranded structure. In certain embodiments, a chemical bond may be induced by ultraviolet light.

In yet another embodiment, the nucleotides at one or both of the two single strands may be modified to prevent or inhibit the activation of cellular enzymes, for example certain nucleases. Techniques for inhibiting the activation of cellular enzymes are known in the art including, but not limited to, 2′-amino modifications, 2′-amino sugar modifications, 2′-F sugar modifications, 2′-F modifications, 2′-alkyl sugar modifications, uncharged backbone modifications, morpholino modifications, 2′-O-methyl modifications, and phosphoramidate (see, e.g., Wagner, Nat. Med. (1995) 1:1116-8). Thus, at least one 2′-hydroxyl group of the nucleotides on a dsRNA is replaced by a chemical group, preferably by a 2′-amino or a 2′-methyl group. Also, at least one nucleotide may be modified to form a locked nucleotide. Such locked nucleotide contains a methylene bridge that connects the 2′-oxygen of ribose with the 4′-carbon of ribose. Introduction of a locked nucleotide into an oligonucleotide improves the affinity for complementary sequences and increases the melting temperature by several degrees.

Modifications of dsRNA molecules provided herein may positively influence their stability in vivo as well as in vitro and also improve their delivery to the (diseased) target side. Furthermore, such structural and chemical modifications may positively influence physiological reactions towards the dsRNA molecules upon administration, e.g. the cytokine release which is preferably suppressed. Such chemical and structural modifications are known in the art and are, inter alia, illustrated in Nawrot (2006) Current Topics in Med Chem, 6, 913-925.

Conjugating a ligand to a dsRNA can enhance its cellular absorption as well as targeting to a particular tissue. In certain instances, a hydrophobic ligand is conjugated to the dsRNA to facilitate direct permeation of the cellular membrane. Alternatively, the ligand conjugated to the dsRNA is a substrate for receptor-mediated endocytosis. These approaches have been used to facilitate cell permeation of antisense oligonucleotides. For example, cholesterol has been conjugated to various antisense oligonucleotides resulting in compounds that are substantially more active compared to their non-conjugated analogs. See M. Manoharan Antisense & Nucleic Acid Drug Development 2002, 12, 103. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. dsRNA compounds bearing folic acid would be efficiently transported into the cell via the folate-receptor-mediated endocytosis. Attachment of folic acid to the 3′-terminus of an oligonucleotide results in increased cellular uptake of the oligonucleotide (Li, S.; Deshmukh, H. M.; Huang, L. Pharm. Res. 1998, 15, 1540). Other ligands that have been conjugated to oligonucleotides include polyethylene glycols, carbohydrate clusters, cross-linking agents, porphyrin conjugates, and delivery peptides.

In certain instances, conjugation of a cationic ligand to oligonucleotides often results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed throughout the oligonucleotide. See M. Manoharan Antisense & Nucleic Acid Drug Development 2002, 12, 103 and references therein.

The ligand-conjugated dsRNA of the invention may be synthesized by the use of a dsRNA that bears a pendant reactive functionality, such as that derived from the attachment of a linking molecule onto the dsRNA. This reactive oligonucleotide may be reacted directly with commercially-available ligands, ligands that are synthesized bearing any of a variety of protecting groups, or ligands that have a linking moiety attached thereto. The methods of the invention facilitate the synthesis of ligand-conjugated dsRNA by the use of, in some preferred embodiments, nucleoside monomers that have been appropriately conjugated with ligands and that may further be attached to a solid-support material. Such ligand-nucleoside conjugates, optionally attached to a solid-support material, are prepared according to some preferred embodiments of the methods of the invention via reaction of a selected serum-binding ligand with a linking moiety located on the 5′ position of a nucleoside or oligonucleotide. In certain instances, an dsRNA bearing an aralkyl ligand attached to the 3′-terminus of the dsRNA is prepared by first covalently attaching a monomer building block to a controlled-pore-glass support via a long-chain aminoalkyl group. Then, nucleotides are bonded via standard solid-phase synthesis techniques to the monomer building-block bound to the solid support. The monomer building block may be a nucleoside or other organic compound that is compatible with solid-phase synthesis.

The dsRNA used in the conjugates of the invention may be conveniently and routinely made through the well-known technique of solid-phase synthesis. It is also known to use similar techniques to prepare other oligonucleotides, such as the phosphorothioates and alkylated derivatives.

Teachings regarding the synthesis of particular modified oligonucleotides may be found in the following U.S. Pat. No. 5,218,105, drawn to polyamine conjugated oligonucleotides; U.S. Pat. Nos. 5,541,307, drawn to oligonucleotides having modified backbones; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. No. 5,587,361 drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,608,046, both drawn to conjugated 4′-desmethyl nucleoside analogs; U.S. Pat. No. 5,610,289, drawn to backbone-modified oligonucleotide analogs; U.S. Pat. No. 6,262,241 drawn to, inter alia, methods of synthesizing 2′-fluoro-oligonucleotides.

In the ligand-conjugated dsRNA and ligand-molecule bearing sequence-specific linked nucleosides of the invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. Oligonucleotide conjugates bearing a variety of molecules such as steroids, vitamins, lipids and reporter molecules, has previously been described (see Manoharan et al., PCT Application WO 93/07883). In a preferred embodiment, the oligonucleotides or linked nucleosides of the invention are synthesized by an automated synthesizer using phosphoramidites derived from ligand-nucleoside conjugates in addition to commercially available phosphoramidites.

The incorporation of a 2′-O-methyl, 2′-O-ethyl, 2′-O-propyl, 2′-O-allyl, 2′-O-aminoalkyl or 2′-deoxy-2′-fluoro group in nucleosides of an oligonucleotide confers enhanced hybridization properties to the oligonucleotide. Further, oligonucleotides containing phosphorothioate backbones have enhanced nuclease stability. Thus, functionalized, linked nucleosides of the invention can be augmented to include either or both a phosphorothioate backbone or a 2′-β-methyl, 2′-O-ethyl, 2′-O-propyl, 2′-O-aminoalkyl, 2′-O-allyl or 2′-deoxy-2′-fluoro group.

In some preferred embodiments, functionalized nucleoside sequences of the invention possessing an amino group at the 5′-terminus are prepared using a DNA synthesizer, and then reacted with an active ester derivative of a selected ligand. Active ester derivatives are well known to those skilled in the art. Representative active esters include N-hydrosuccinimide esters, tetrafluorophenolic esters, pentafluorophenolic esters and pentachlorophenolic esters. The reaction of the amino group and the active ester produces an oligonucleotide in which the selected ligand is attached to the 5′-position through a linking group. The amino group at the 5′-terminus can be prepared utilizing a 5′-Amino-Modifier C6 reagent. In a preferred embodiment, ligand molecules may be conjugated to oligonucleotides at the 5′-position by the use of a ligand-nucleoside phosphoramidite wherein the ligand is linked to the 5′-hydroxy group directly or indirectly via a linker Such ligand-nucleoside phosphoramidites are typically used at the end of an automated synthesis procedure to provide a ligand-conjugated oligonucleotide bearing the ligand at the 5′-terminus.

In one preferred embodiment of the methods of the invention, the preparation of ligand conjugated oligonucleotides commences with the selection of appropriate precursor molecules upon which to construct the ligand molecule. Typically, the precursor is an appropriately-protected derivative of the commonly-used nucleosides. For example, the synthetic precursors for the synthesis of the ligand-conjugated oligonucleotides of the invention include, but are not limited to, 2′-aminoalkoxy-5′-ODMT-nucleosides, 2′-6-aminoalkylamino-5′-ODMT-nucleosides, 5′-6-aminoalkoxy-2′-deoxy-nucleosides, 5′-6-aminoalkoxy-2-protected-nucleosides, 3′-6-aminoalkoxy-5′-ODMT-nucleosides, and 3′-aminoalkylamino-5′-ODMT-nucleosides that may be protected in the nucleobase portion of the molecule. Methods for the synthesis of such amino-linked protected nucleoside precursors are known to those of ordinary skill in the art.

In many cases, protecting groups are used during the preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y., 1991.

Amino-protecting groups stable to acid treatment are selectively removed with base treatment, and are used to make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p. 1) and various substituted sulfonylethyl carbamates exemplified by the Nsc group (Samukov et al., Tetrahedron Lett., 1994, 35:7821.

Additional amino-protecting groups include, but are not limited to, carbamate protecting groups, such as 2-trimethylsilylethoxycarbonyl (Teoc), 1-methyl-144-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl. Equivalents of these amino-protecting groups are also encompassed by the compounds and methods of the invention.

Many solid supports are commercially available and one of ordinary skill in the art can readily select a solid support to be used in the solid-phase synthesis steps. In certain embodiments, a universal support is used. A universal support allows for preparation of oligonucleotides having unusual or modified nucleotides located at the 3′-terminus of the oligonucleotide. For further details about universal supports see Scott et al., Innovations and Perspectives in solid-phase Synthesis, 3rd International Symposium, 1994, Ed. Roger Epton, Mayflower Worldwide, 115-124]. In addition, it has been reported that the oligonucleotide can be cleaved from the universal support under milder reaction conditions when oligonucleotide is bonded to the solid support via a syn-1,2-acetoxyphosphate group which more readily undergoes basic hydrolysis. See Guzaev, A. I.; Manoharan, M. J. Am. Chem. Soc. 2003, 125, 2380.

The nucleosides are linked by phosphorus-containing or non-phosphorus-containing covalent internucleoside linkages. For the purposes of identification, such conjugated nucleosides can be characterized as ligand-bearing nucleosides or ligand-nucleoside conjugates. The linked nucleosides having an aralkyl ligand conjugated to a nucleoside within their sequence will demonstrate enhanced dsRNA activity when compared to like dsRNA compounds that are not conjugated.

The aralkyl-ligand-conjugated oligonucleotides of the invention also include conjugates of oligonucleotides and linked nucleosides wherein the ligand is attached directly to the nucleoside or nucleotide without the intermediacy of a linker group. The ligand may preferably be attached, via linking groups, at a carboxyl, amino or oxo group of the ligand. Typical linking groups may be ester, amide or carbamate groups.

Specific examples of preferred modified oligonucleotides envisioned for use in the ligand-conjugated oligonucleotides of the invention include oligonucleotides containing modified backbones or non-natural internucleoside linkages. As defined here, oligonucleotides having modified backbones or internucleoside linkages include those that retain a phosphorus atom in the backbone and those that do not have a phosphorus atom in the backbone. For the purposes of the invention, modified oligonucleotides that do not have a phosphorus atom in their intersugar backbone can also be considered to be oligonucleosides.

Specific oligonucleotide chemical modifications are described below. It is not necessary for all positions in a given compound to be uniformly modified. Conversely, more than one modifications may be incorporated in a single dsRNA compound or even in a single nucleotide thereof.

Preferred modified internucleoside linkages or backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free-acid forms are also included. Teachings relating to the preparation of the above phosphorus-atom-containing linkages are well known in the art.

Preferred modified internucleoside linkages or backbones that do not include a phosphorus atom therein (i.e., oligonucleosides) have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents relating to the preparation of the above oligonucleosides include, but are not limited to, U.S. Pat. Nos. 5,034,506; 5,214,134; 5,216,141; 5,264,562; 5,466,677; 5,470,967; 5,489,677; 5,602,240 and 5,663,312, each of which is herein incorporated by reference.

In other preferred oligonucleotide mimetics, both the sugar and the internucleoside linkage, i.e., the backbone, of the nucleoside units are replaced with novel groups. The nucleobase units are maintained for hybridization with an appropriate nucleic acid target compound. One such oligonucleotide, an oligonucleotide mimetic, that has been shown to have excellent hybridization properties, is referred to as a peptide nucleic acid (PNA). In PNA compounds, the sugar-backbone of an oligonucleotide is replaced with an amide-containing backbone, in particular an aminoethylglycine backbone. The nucleobases are retained and are bound directly or indirectly to atoms of the amide portion of the backbone. Teaching of PNA compounds can be found for example in U.S. Pat. No. 5,539,082.

Some preferred embodiments of the invention employ oligonucleotides with phosphorothioate linkages and oligonucleosides with heteroatom backbones, and in particular —CH₂—NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

The oligonucleotides employed in the ligand-conjugated oligonucleotides of the invention may additionally or alternatively comprise nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligonucleotides of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2° C. (Id., pages 276-278) and are presently preferred base substitutions, even more particularly when combined with 2′-methoxyethyl sugar modifications.

Representative United States patents relating to the preparation of certain of the above-noted modified nucleobases as well as other modified nucleobases include, but are not limited to, the above noted U.S. Pat. No. 3,687,808, as well as U.S. Pat. Nos. 5,134,066; 5,459,255; 5,552,540; 5,594,121 and 5,596,091 all of which are hereby incorporated by reference.

In certain embodiments, the oligonucleotides employed in the ligand-conjugated oligonucleotides of the invention may additionally or alternatively comprise one or more substituted sugar moieties. Preferred oligonucleotides comprise one of the following at the 2′ position: OH; F; O-, S-, or N-alkyl, O-, S-, or N-alkenyl, or O, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy [2′-O—CH₂CH₂OCH₃, also known as 2′-O-(2-methoxyethyl) or 2′-MOE], i.e., an alkoxyalkoxy group. A further preferred modification includes 2′-dimethylaminooxyethoxy, i.e., a O(CH₂)₂ON(CH₃)₂ group, also known as 2′-DMAOE, as described in U.S. Pat. No. 6,127,533, filed on Jan. 30, 1998, the contents of which are incorporated by reference.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides.

As used herein, the term “sugar substituent group” or “2′-substituent group” includes groups attached to the 2′-position of the ribofuranosyl moiety with or without an oxygen atom. Sugar substituent groups include, but are not limited to, fluoro, O-alkyl, O-alkylamino, O-alkylalkoxy, protected O-alkylamino, O-alkylaminoalkyl, O-alkyl imidazole and polyethers of the formula (O-alkyl)ₘ, wherein m is 1 to about 10. Preferred among these polyethers are linear and cyclic polyethylene glycols (PEGs), and (PEG)-containing groups, such as crown ethers and, inter alia, those which are disclosed by Delgardo et. al. (Critical Reviews in Therapeutic Drug Carrier Systems 1992, 9:249), which is hereby incorporated by reference in its entirety. Further sugar modifications are disclosed by Cook (Anti-fibrosis Drug Design, 1991, 6:585-607). Fluoro, O-alkyl, O-alkylamino, O-alkyl imidazole, O-alkylaminoalkyl, and alkyl amino substitution is described in U.S. Pat. No. 6,166,197, entitled “Oligomeric Compounds having Pyrimidine Nucleotide(s) with 2′ and 5′ Substitutions,” hereby incorporated by reference in its entirety.

Additional sugar substituent groups amenable to the invention include 2′-SR and 2′-NR₂ groups, wherein each R is, independently, hydrogen, a protecting group or substituted or unsubstituted alkyl, alkenyl, or alkynyl. 2′-SR Nucleosides are disclosed in U.S. Pat. No. 5,670,633, hereby incorporated by reference in its entirety. The incorporation of 2′-SR monomer synthons is disclosed by Hamm et al. (J. Org. Chem., 1997, 62:3415-3420). 2′-NR nucleosides are disclosed by Goettingen, M., J. Org. Chem., 1996, 61, 6273-6281; and Polushin et al., Tetrahedron Lett., 1996, 37, 3227-3230. Further representative 2′-substituent groups amenable to the invention include those having one of formula I or II:

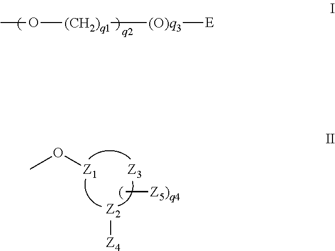

wherein,
E is C₁-C₁₀ alkyl, N(Q₃)(Q₄) or N═C (Q₃)(Q₄); each Q₃ and Q₄ is, independently, H, C₁-C₁₀ alkyl, dialkylaminoalkyl, a nitrogen protecting group, a tethered or untethered conjugate group, a linker to a solid support; or Q₃ and Q₄, together, form a nitrogen protecting group or a ring structure optionally including at least one additional heteroatom selected from N and O;
q₁ is an integer from 1 to 10;
q₂ is an integer from 1 to 10;
q₃ is 0 or 1;
q₄ is 0, 1 or 2;
each Z₁, Z₂ and Z₃ is, independently, C₄-C₇ cycloalkyl, C₅-C₁₄ aryl or C₃-C₁₅ heterocyclyl, wherein the heteroatom in said heterocyclyl group is selected from oxygen, nitrogen and sulfur;
Z₄ is OM₁, SM₁, or N(M₁)₂; each M₁ is, independently, H, C₁-C₈ alkyl, C₁-C₈ haloalkyl, C(═NH)N(H)M₂, C(═O)N(H)M₂ or OC(═O)N(H)M₂; M₂ is H or C₁-C₈ alkyl; and
Z₅ is C₁-C₁₀ alkyl, C₁-C₁₀ haloalkyl, C₂-C₁₀ alkenyl, C₂-C₁₀ alkynyl, C₆-C₁₄ aryl, N(Q₃)(Q₄), OQ₃, halo, SQ₃ or CN.

Representative 2′-O-sugar substituent groups of formula I are disclosed in U.S. Pat. No. 6,172,209, entitled “Capped 2′-Oxyethoxy Oligonucleotides,” hereby incorporated by reference in its entirety. Representative cyclic 2′-O-sugar substituent groups of formula II are disclosed in U.S. Pat. No. 6,271,358, entitled “RNA Targeted 2′-Modified Oligonucleotides that are Conformationally Preorganized,” hereby incorporated by reference in its entirety.

Sugars having O-substitutions on the ribosyl ring are also amenable to the invention. Representative substitutions for ring O include, but are not limited to, S, CH₂, CHF, and CF₂. Oligonucleotides may also have sugar mimetics, such as cyclobutyl moieties, in place of the pentofuranosyl sugar. Representative United States patents relating to the preparation of such modified sugars include, but are not limited to, U.S. Pat. Nos. 5,359,044; 5,466,786; 5,519,134; 5,591,722; 5,597,909; 5,646,265 and 5,700,920, all of which are hereby incorporated by reference.

Additional modifications may also be made at other positions on the oligonucleotide, particularly the 3′ position of the sugar on the 3′ terminal nucleotide. For example, one additional modification of the ligand-conjugated oligonucleotides of the invention involves chemically linking to the oligonucleotide one or more additional non-ligand moieties or conjugates which enhance the activity, cellular distribution or cellular uptake of the oligonucleotide. Such moieties include but are not limited to lipid moieties, such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-5-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

The invention also includes compositions employing oligonucleotides that are substantially chirally pure with regard to particular positions within the oligonucleotides. Examples of substantially chirally pure oligonucleotides include, but are not limited to, those having phosphorothioate linkages that are at least 75% Sp or Rp (Cook et al., U.S. Pat. No. 5,587,361) and those having substantially chirally pure (Sp or Rp) alkylphosphonate, phosphoramidate or phosphotriester linkages (Cook, U.S. Pat. Nos. 5,212,295 and 5,521,302).

In certain instances, the oligonucleotide may be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Typical conjugation protocols involve the synthesis of oligonucleotides bearing an aminolinker at one or more positions of the sequence. The amino group is then reacted with the molecule being conjugated using appropriate coupling or activating reagents. The conjugation reaction may be performed either with the oligonucleotide still bound to the solid support or following cleavage of the oligonucleotide in solution phase. Purification of the oligonucleotide conjugate by HPLC typically affords the pure conjugate.

Alternatively, the molecule being conjugated may be converted into a building block, such as a phosphoramidite, via an alcohol group present in the molecule or by attachment of a linker bearing an alcohol group that may be phosphorylated.

Importantly, each of these approaches may be used for the synthesis of ligand conjugated oligonucleotides. Amino linked oligonucleotides may be coupled directly with ligand via the use of coupling reagents or following activation of the ligand as an NHS or pentfluorophenolate ester. Ligand phosphoramidites may be synthesized via the attachment of an aminohexanol linker to one of the carboxyl groups followed by phosphitylation of the terminal alcohol functionality. Other linkers, such as cysteamine, may also be utilized for conjugation to a chloroacetyl linker present on a synthesized oligonucleotide.

The person skilled in the art is readily aware of methods to introduce the molecules of this invention into cells, tissues or organisms. Corresponding examples have also been provided in the detailed description of the invention above. For example, the nucleic acid molecules or the vectors of this invention, encoding for at least one strand of the inventive dsRNAs may be introduced into cells or tissues by methods known in the art, like transfections etc.

Also for the introduction of dsRNA molecules, means and methods have been provided For example, targeted delivery by glycosylated and folate-modified molecules, including the use of polymeric carriers with ligands, such as galactose and lactose or the attachment of folic acid to various macromolecules allows the binding of molecules to be delivered to folate receptors. Targeted delivery by peptides and proteins other than antibodies, for example, including RGD-modified nanoparticles to deliver siRNA in vivo or multicomponent (nonviral) delivery systems including short cyclodextrins, adamantine-PEG are known. Yet, also the targeted delivery using antibodies or antibody fragments, including (monovalent) Fab-fragments of an antibody (or other fragments of such an antibody) or single-chain antibodies are envisaged. Injection approaches for target directed delivery comprise, inter alia, hydrodynamic i.v. injection. Also cholesterol conjugates of dsRNA may be used for targeted delivery, whereby the conjugation to lipohilic groups enhances cell uptake and improve pharmacokinetics and tissue biodistribution of oligonucleotides. Also cationic delivery systems are known, whereby synthetic vectors with net positive (cationic) charge to facilitate the complex formation with the polyanionic nucleic acid and interaction with the negatively charged cell membrane. Such cationic delivery systems comprise also cationic liposomal delivery systems, cationic polymer and peptide delivery systems. Other delivery systems for the cellular uptake of dsRNA/siRNA are aptamer-ds/siRNA. Also gene therapy approaches can be used to deliver the inventive dsRNA molecules or nucleic acid molecules encoding the same. Such systems comprise the use of non-pathogenic virus, modified viral vectors, as well as deliveries with nanoparticles or liposomes. Other delivery methods for the cellular uptake of dsRNA are extracorporeal, for example ex vivo treatments of cells, organs or tissues. Certain of these technologies are described and summarized in publications, like Akhtar (2007), Journal of Clinical Investigation 117, 3623-3632, Nguyen et al. (2008), Current Opinion in Moleculare Therapeutics 10, 158-167, Zamboni (2005), Clin Cancer Res 11, 8230-8234 or Ikeda et al. (2006), Pharmaceutical Research 23, 1631-1640

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The above provided embodiments and items of the present invention are now illustrated with the following, non-limiting examples.

### DESCRIPTION OF FIGURES AND APPENDED TABLES

Table 1—dsRNA targeting human IL-18 gene without modifications. Letters in capitals represent RNA nucleotides.

Table 2—dsRNA targeting human IL-18 gene with modifications. Letters in capitals represent RNA nucleotides, lower case letters “c”, “g”, “a” and “u” represent 2′ O-methyl-modified nucleotides, “s” represents phosphorothioate, “dT” deoxythymidine, “OMedT” 5′-O-methyl-deoxythymidine, “p” 5′-phosphate group at 5′ end of respective dsRNA strand, and “f” 2′-fluoro modification of nucleotide represented by preceding symbol.

Table 3—Characterization of dsRNAs targeting human IL-18: Activity testing for dose response in HCT-116 cells. IC 50: 50% inhibitory concentration, IC 80: 80% inhibitory concentration, IC 20: 20% inhibitory concentration.

Table 4—Characterization of dsRNAs targeting human IL-18: Stability and Cytokine Induction. t ½: half-life of a strand as defined in examples, PBMC: Human peripheral blood mononuclear cells.

Table 5—dsRNA targeting murine IL-18 gene without modifications. Letters in capitals represent RNA nucleotides.

Table 6—dsRNA targeting murine IL-18 gene with modifications. Letters in capitals represent RNA nucleotides, lower case letters “c”, “g”, “a” and “u” represent 2′ O-methyl-modified nucleotides, “s” represents phosphorothioate and “dT” deoxythymidine.

Table 7—Characterization of dsRNAs targeting murine IL-18: Activity testing for dose response in RAW264.7 cells. IC 50: 50% inhibitory concentration, IC 80: 80% inhibitory concentration, IC 20: 20% inhibitory concentration. Stability and Cytokine Induction. t ½: half-life of a strand as defined in examples, PBMC: Human peripheral blood mononuclear cells.

Table 8—Sequences of bDNA probes for determination of human IL-18. LE=label extender, CE=capture extender, BL=blocking probe.

Table 9—Sequences of bDNA probes for determination of human GAPDH. LE=label extender, CE=capture extender, BL=blocking probe.

Table 10—Sequences of bDNA probes for determination of murine IL-18. LE=label extender, CE=capture extender, BL=blocking probe.

Table 11—Sequences of bDNA probes for determination of murine GAPDH. LE=label extender, CE=capture extender, BL=blocking probe.

Table 12—dsRNA targeting human IL-18 gene without modifications and their modified counterparts. Letters in capitals represent RNA nucleotides, lower case letters “c”, “g”, “a” and “u” represent 2′ O-methyl-modified nucleotides, “s” represents phosphorothioate “dT” deoxythymidine, “5′-OMedT” 5′-O-methyl-deoxythymidine, “p” 5′-phosphate group at 5′ end of respective dsRNA strand, and “f” 2′-fluoro modification of nucleotide represented by preceding symbol.

Table 13—dsRNA targeting murine IL-18 gene without modifications and their modified counterparts. Letters in capitals represent RNA nucleotides, lower case letters “c”, “g”, “a” and “u” represent 2′ O-methyl-modified nucleotides, “s” represents phosphorothioate “dT” deoxythymidine.

Table 14—Bioinformatic Selection of Potential Off Targets

Table 15—Perfect Matching siRNAs for Predicted Off Target Sites

Table 16—Potential off-target target sites, mismatch locations and (on)off-target activity

Table 17—Dose Response Analysis for Selected Off Targets in A431 Cells

Table 18—bDNA Probeset for endogeneous Analysis of dsRNA comprising sequence pair 239/240 OFF-1

Table 19—bDNA Probeset for endogeneous Analysis of dsRNA comprising sequence pair 239/240 OFF-7

Table 20—bDNA Probeset for endogeneous Analysis of dsRNA comprising sequence pair 255/256 OFF-1

Table 21—bDNA Probeset for endogeneous Analysis of dsRNA comprising sequence pair 255/256 OFF-7

Table 22—bDNA Probeset for endogeneous Analysis of dsRNA comprising sequence pair 255/256 OFF-13

Table 23—bDNA Probeset for endogeneous Analysis of dsRNA comprising sequence pair 165/166 OFF-2

Table 24—bDNA Probeset for endogeneous Analysis of dsRNA comprising sequence pair 165/166 OFF-7

Table 25—bDNA Probeset for endogeneous Analysis of dsRNA comprising sequence pair 165/166 OFF-12

Table 26—bDNA Probeset for endogeneous Analysis of human IL-18

Table 27—bDNA Probeset for endogeneous Analysis of human GAPDH

FIG. 1—dsRNAs targeting human IL-18 gene (“IL-18 siRNA”) inhibit LPS-induced IL-18 protein secretion and mRNA expression in THP-1 cells.

### EXAMPLES

### Identification of dsRNAs for Therapeutic Use

dsRNA design was carried out to identify dsRNAs specifically targeting human IL-18 for therapeutic use. First, the known mRNA sequence of human (Homo sapiens) IL-18 (NM_{—}001562.2 listed as SEQ ID NO. 783) was downloaded from NCBI Genbank.

In identifying RNAi agents, the selection was limited to 19 mer sequences having at least 2 mismatches to any other sequence in the human RefSeq database (release 28), which we assumed to represent the comprehensive human transcriptome, by using a proprietary algorithm.

The coding sequence (CDS) of the cynomolgous monkey (Macaca fascicularis) IL-18 gene was sequenced (see SEQ ID NO. 784) and examined by computer analysis for target regions of RNAi agents.

All sequences containing 4 or more consecutive G's (poly-G sequences) were excluded from the synthesis.

The sequences thus identified formed the basis for the synthesis of the RNAi agents in appended Tables 1 and 2. The relation between unmodified sequences shown in table 1 and its modified counterparts of table 2 is shown in table 12. dsRNAs cross-reactive to human as well as cynomolgous monkey IL-18 were defined as most preferable for therapeutic use.

Identification of Murine dsRNAs targeting IL-18

dsRNA design was carried out to identify dsRNAs specifically targeting murine IL-18 for prove-of-concept. The known mRNA sequence of mouse (Mus musculus) IL-18 (NM 008360.1 listed as SEQ ID NO. 785) was downloaded from NCBI Genbank.

In identifying RNAi agents, the selection was limited to 19 mer sequences having at least 2 mismatches to any other sequence in the mouse RefSeq database (release 28), which we assumed to represent the comprehensive mouse transcriptome, by using a proprietary algorithm.

All sequences containing 4 or more consecutive G's (poly-G sequences) were excluded from the synthesis.

The sequences thus identified formed the basis for the synthesis of the RNAi agents in appended tables 5 and 6 dsRNAs cross-reactive to mouse IL-18. The relation between unmodified sequences shown in table 5 and its modified counterparts of table 6 is shown in table 13.

dsRNA Synthesis

Where the source of a reagent is not specifically given herein, such reagent may be obtained from any supplier of reagents for molecular biology at a quality/purity standard for application in molecular biology.

Single-stranded RNAs were produced by solid phase synthesis on a scale of 1 μmole using an Expedite 8909 synthesizer (Applied Biosystems, Applera Deutschland GmbH, Darmstadt, Germany) and controlled pore glass (CPG, 500 Å, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleiβheim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20° C. until use.

Activity Testing of Therapeutic dsRNAs Targeting IL-18

The activity of the dsRNAs directed to IL-18 was tested in HCT-116 cells.

HCT-116 cells in culture were used for quantitation of IL-18 mRNA by branched DNA in total mRNA isolated from cells incubated with IL-18 specific dsRNAs assay.

HCT-116 cells were obtained from American Type Culture Collection (Rockville, Md., cat. No. CCL-247) and cultured in McCoy's 5a medium (Biochrom AG, Berlin, Germany, cat. No. F 1015) supplemented to contain 10% fetal calf serum (FCS) (Biochrom AG, Berlin, Germany, cat. No. S0115), 2 mM L-Glutamin (Biochrom AG, Berlin, Germany, cat. No. K0283) and Penicillin 100 U/ml, Streptomycin 100 mg/ml (Biochrom AG, Berlin, Germany, cat. No. A2213) at 37° C. in an atmosphere with 5% CO₂ in a humidified incubator (Heraeus HERAcell, Kendro Laboratory Products, Langenselbold, Germany).

Transfection of dsRNA was performed directly after seeding 20,000 cells/well on a 96-well plate, and was carried out with Lipofectamine 2000 (Invitrogen GmbH, Karlsruhe, Germany, cat. No. 11668-019) as described by the manufacturer. In two independent single dose experiments performed in quadruplicates dsRNAs were transfected at a concentration of 50 nM. Most effective dsRNAs against IL-18 from the single dose screens were further characterized by dose response curves. For dose response curves, transfections were performed as for the single dose screen above, but with concentrations starting with 100 nM and decreasing in 6-fold dilutions down to 10 fM. After transfection cells were incubated for 24 h at 37° C. and 5% CO₂ in a humidified incubator (Heraeus GmbH, Hanau, Germany). For measurement of IL-18 mRNA cells were harvested and lysed at 53° C. following procedures recommended by the manufacturer of the Quantigene Explore Kit (Panomics, Fremont, Calif., USA, cat. No. QG0004) for bDNA. Afterwards, 50 μl of the lysates were incubated with probesets specific to human IL-18 and 10 μl of the lysates for human GAPDH (sequences of probesets see appended tables 8 and 9) and processed according to the manufacturer's protocol for QuantiGene. Chemoluminescence was measured in a Victor2-Light (Perkin Elmer, Wiesbaden, Germany) as RLUs (relative light units) and values obtained with the human IL-18 probeset were normalized to the respective human GAPDH values for each well and in then related to the mean of three unrelated control dsRNAs in the single dose experiments, whereas in the dose response experiments the values obtained with the specific siRNAs where related to the mock transfection (=Lipofectamine-2000 without dsRNA). Inhibition data are given in appended tables 2 and 3.

The activity of the siRNAs directed to murine IL-18 was tested in RAW264.7 cells. Inhibition data are given in appended table 6.

Activity Testing of dsRNAs Targeting Murine IL-18

RAW264.7 cells in culture were used for quantitation of murine IL-18 mRNA by branched DNA in total mRNA isolated from cells incubated with murine IL-18 specific siRNAs assay.

RAW264.7 cells were obtained from American Type Culture Collection (Rockville, Md., cat. No. TIB-71) and cultured in DMEM (Biochrom AG, Berlin, Germany, cat. No. F0435) supplemented to contain 10% fetal calf serum (FCS) (Biochrom AG, Berlin, Germany, cat. No. S0115), 2 mM L-Glutamin (Biochrom AG, Berlin, Germany, cat. No. K0283), 1 mM Sodiumpyruvate (Biochrom AG, Berlin, Germany, cat. No. L0473) and Penicillin 100 U/ml, Streptomycin 100 mg/ml (Biochrom AG, Berlin, Germany, cat. No. A2213) at 37° C. in an atmosphere with 5% CO₂ in a humidified incubator (Heraeus HERAcell, Kendro Laboratory Products, Langenselbold, Germany).

About 16 hours before transfection, IL-18 expression was induced by adding Interferon-gamma (Sigma-Aldrich, Taufkirchen, Germany, cat. No. 14777) to the cell culture medium at a final concentration of 10 ng/ml. Transfection of siRNA was performed directly after seeding 20,000 cells/well on a 96-well plate, and was carried out with HiPerFect (Qiagen, Hilden, Germany, cat. No. 301705) as described by the manufacturer. In two independent single dose experiments performed in quadruplicates siRNAs were transfected at a concentration of 50 nM. Most effective siRNAs against murine IL-18 from the single dose screens were further characterized by dose response curves. For dose response curves, transfections were performed as for the single dose screen above, but with concentrations starting with 100 nM and decreasing in 6-fold dilutions down to 10 fM. After transfection cells were incubated for 24 h at 37° C. and 5% CO₂ in a humidified incubator (Heraeus GmbH, Hanau, Germany). For measurement of murine IL-18 mRNA cells were harvested and lysed at 53° C. following procedures recommended by the manufacturer of the Quantigene II Explore Kit (Panomics, Fremont, Calif., USA, cat. No. QS9900) for bDNA. Afterwards, 50 μA of the lysates were incubated with probesets specific to murine IL-18 and 10 μA of the lysates for murine GAPDH (sequences of probesets see appended tables 10 and 11) and processed according to the manufacturer's protocol for QuantiGene. Chemoluminescence was measured in a Victor2-Light (Perkin Elmer, Wiesbaden, Germany) as RLUs (relative light units) and values obtained with the human murine IL-18 probeset were normalized to the respective human GAPDH values for each well and then related to the mean of three unrelated control siRNAs in the single dose experiments, whereas in the dose response experiments the values obtained with the specific siRNAs where related to the mock transfection (=HiPerFect without siRNA).

Stability of dsRNAs

Stability of dsRNAs targeting human IL-18 was determined in in vitro assays with either human or mouse serum by measuring the half-life of each single strand.

Measurements were carried out in triplicates for each time point, using 3 μl 50 μM dsRNA sample mixed with 30 μl human serum (Sigma) or mouse serum (Sigma). Mixtures were incubated for either 0 min, 30 min, 1 h, 3 h, 6 h, 24 h, or 48 h at 37° C. As control for unspecific degradation dsRNA was incubated with 30 μl 1×PBS pH 6.8 for 48 h. Reactions were stopped by the addition of 4 μl proteinase K (20 mg/ml), 25 μl of “Tissue and Cell Lysis Solution” (Epicentre) and 38 μl Millipore water for 30 min at 65° C. Samples were afterwards spin filtered through a 0.2 μm 96 well filter plate at 1400 rpm for 8 min, washed with 55 μl Millipore water twice and spin filtered again.

For separation of single strands and analysis of remaining full length product (FLP), samples were run through an ion exchange Dionex Summit HPLC under denaturing conditions using as eluent A 20 mM Na3PO4 in 10% ACN pH=11 and for eluent B 1 M NaBr in eluent A.

The following gradient was applied:

| Time | % A | % B |
|---|---|---|
| −1.0 min | 75 | 25 |
| 1.00 min | 75 | 25 |
| 19.0 min | 38 | 62 |
| 19.5 min | 0 | 100 |
| 21.5 min | 0 | 100 |
| 22.0 min | 75 | 25 |
| 24.0 min | 75 | 25 |

For every injection, the chromatograms were integrated automatically by the Dionex Chromeleon 6.60 HPLC software, and were adjusted manually if necessary. All peak areas were corrected to the internal standard (IS) peak and normalized to the incubation at t=0 min. The area under the peak and resulting remaining FLP was calculated for each single strand and triplicate separately. Half-life (t½) of a strand was defined by the average time point [h] for triplicates at which half of the FLP was degraded. Results are given in appended table 4.

### Cytokine Induction

Potential cytokine induction of dsRNAs was determined by measuring the release of IFN-a and TNF-a in an in vitro PBMC assay.

Human peripheral blood mononuclear cells (PBMC) were isolated from buffy coat blood of two donors by Ficoll centrifugation at the day of transfection. Cells were transfected in quadruplicates with dsRNA and cultured for 24 h at 37° C. at a final concentration of 130 nM in Opti-MEM, using either Gene Porter 2 (GP2) or DOTAP. dsRNA sequences that were known to induce IFN-a and TNF-a in this assay, as well as a CpG oligo, were used as positive controls. Chemical conjugated dsRNA or CpG oligonucleotides that did not need a transfection reagent for cytokine induction, were incubated at a concentration of 500 nM in culture medium. At the end of incubation, the quadruplicate culture supernatant were pooled.

IFN-a and TNF-a was then measured in these pooled supernatants by standard sandwich ELISA with two data points per pool. The degree of cytokine induction was expressed relative to positive controls using a score from 0 to 5, with 5 indicating maximum induction. Results are given in appended table 4.

Functional Inhibition by dsRNA

The ability of dsRNAs targeting human IL-18 to mediate a functional response was determined in a cell-based in vitro assay. The functional readout was siRNA-induced inhibition of LPS-stimulated human IL-18 protein secretion from a human macrophage cell line (THP-1).

### Transfection

Briefly, siRNAs targeting human IL-18 and a control siRNA targeting AHSA-1 were transfected into THP-1 cells (ATCC, cat. No. TIB-202) using DharmaFECT 1 (Dharmacon, Inc., cat. No. T-2001-02). The siRNAs targeting human IL-18 and control siRNA were complexed at 10 nM with DharmaFECT 1 for 20 minutes at room temperature. THP-1 cells were plated at a cell density of 100,000 cells per well in a 96-well plate in RPMI 1640 (Invitrogen, cat. No. 118750-93)+0.5% fetal bovine serum (Gemini, cat. No. 100-106). The transfection mix was added to the cells at 0.15 l/well and incubated at 37° C. for 48 hours (for mRNA analysis) or for 66 hours (for IL-18 protein analysis). Complete media was added 3 hours after transfection.

mRNA Analysis

For measurement of mRNAs, transfected THP-1 cells were harvested and lysed at 65° C. following procedures recommended by the manufacturer of the Quantigene II Explore Kit (Panomics, cat. No. QS9900) for bDNA. Afterwards, lysates were incubated with probesets specific for human IL-18 and actin, and processed according to the manufacturer's protocol for Quantigene II. Values obtained with the human IL-18 probeset were normalized to the respective human actin values obtained for each well and then related to the mock transfection (=DharmFECT 1 without siRNA).

### IL-18 Protein Analysis

At 48 hours after transfection, cells were treated with 1 μg/ml LPS (Sigma, cat. No. L-6529) to induce IL-18 expression and secretion (Zeisel et al. (2004) Cell. Microbiol. 6: 593-598). At 18 hours after LPS treatment, culture supernatants were collected and human IL-18 was measured in the supernatants by standard sandwich ELISA (R&D Systems, cat. No. 7620). The concentration of human IL-18 protein was calibrated from a dose response curve based on reference standards provided by the manufacturer.

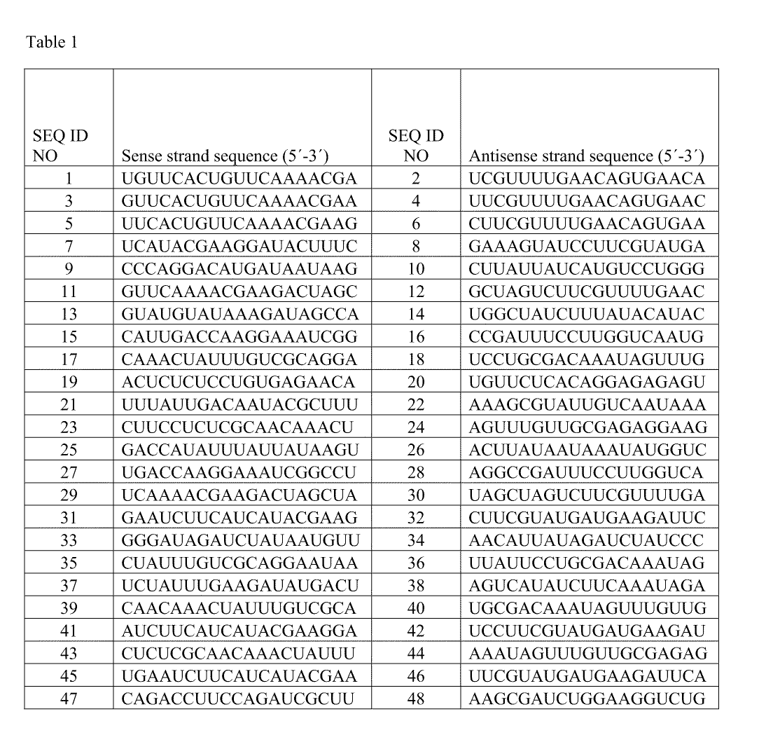

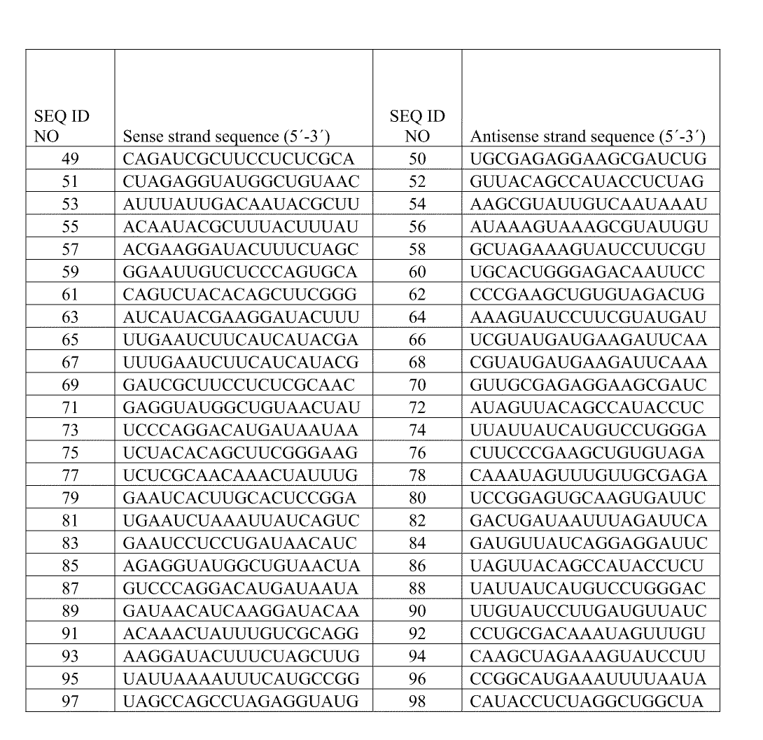

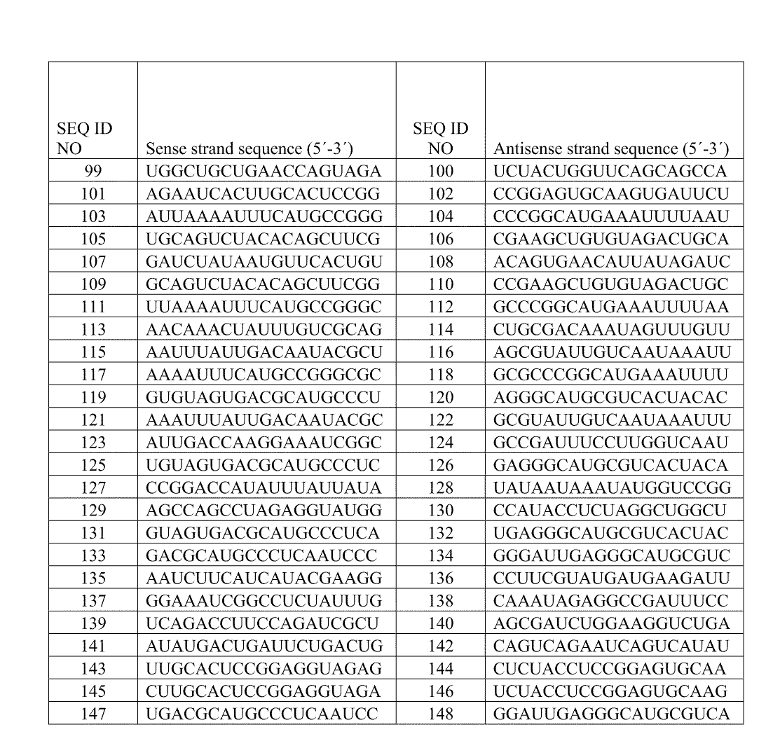

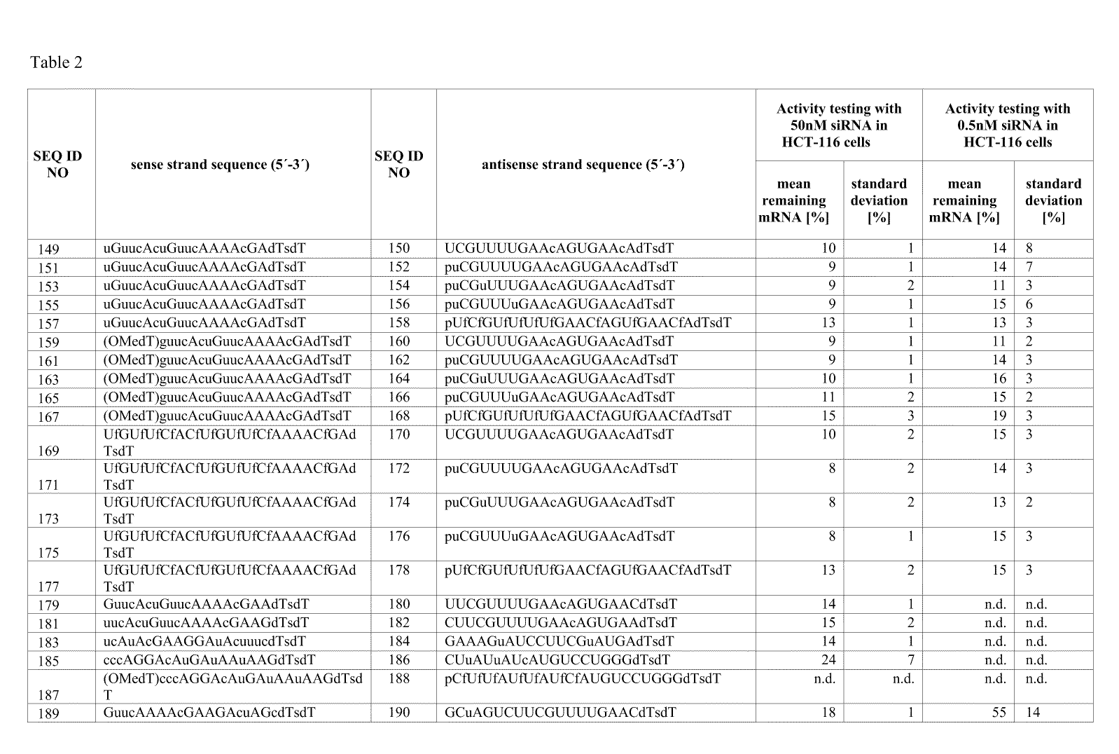

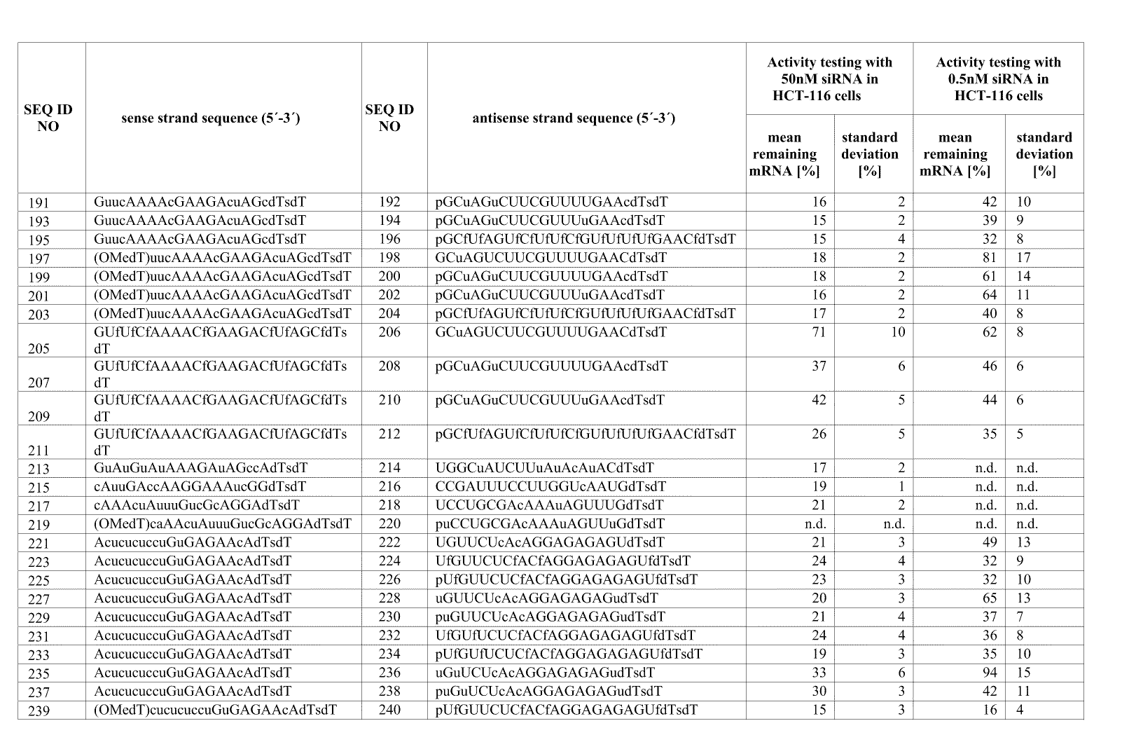

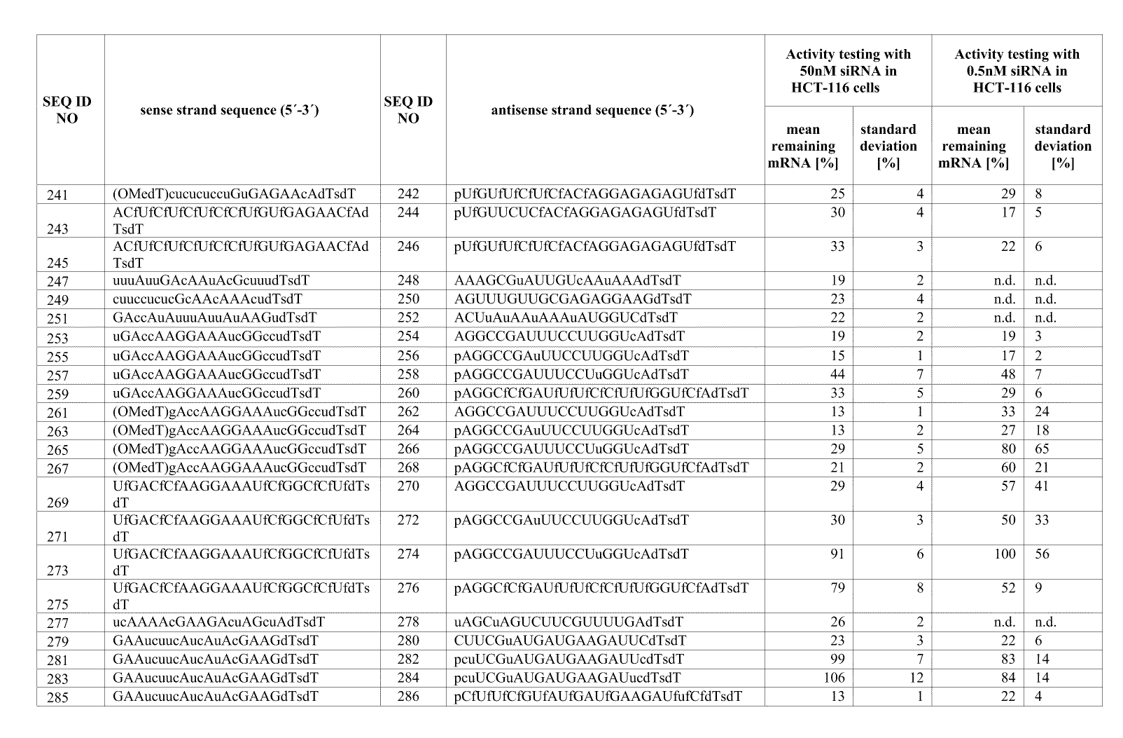

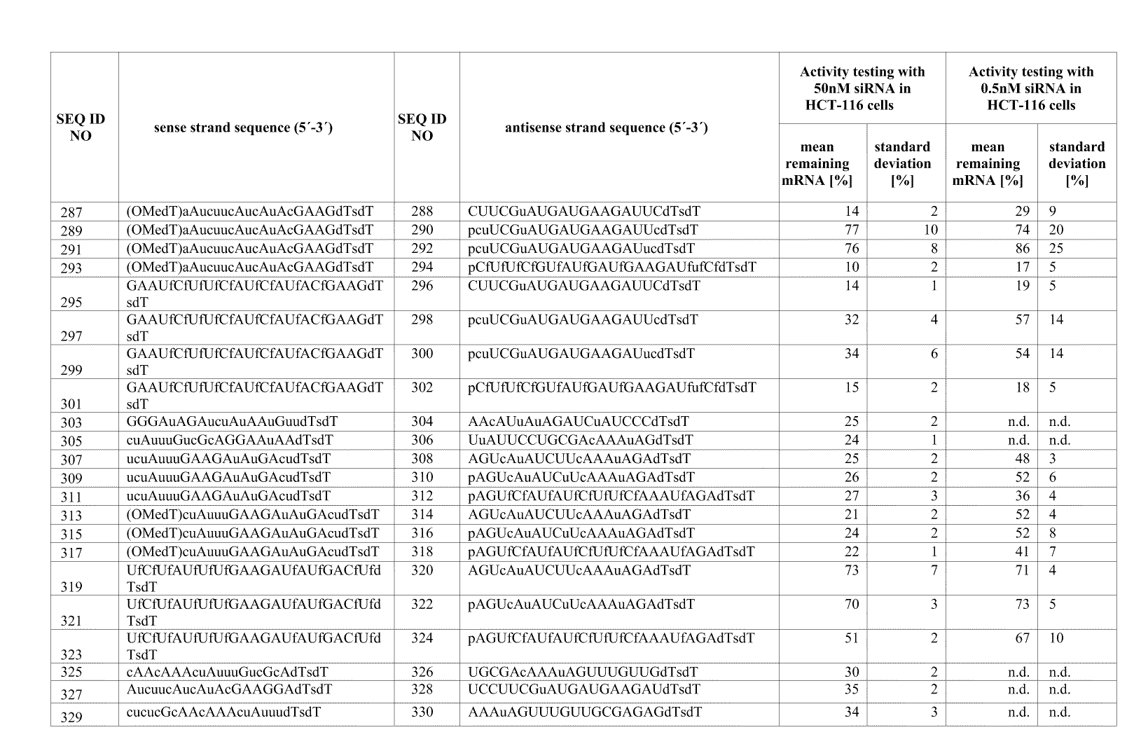

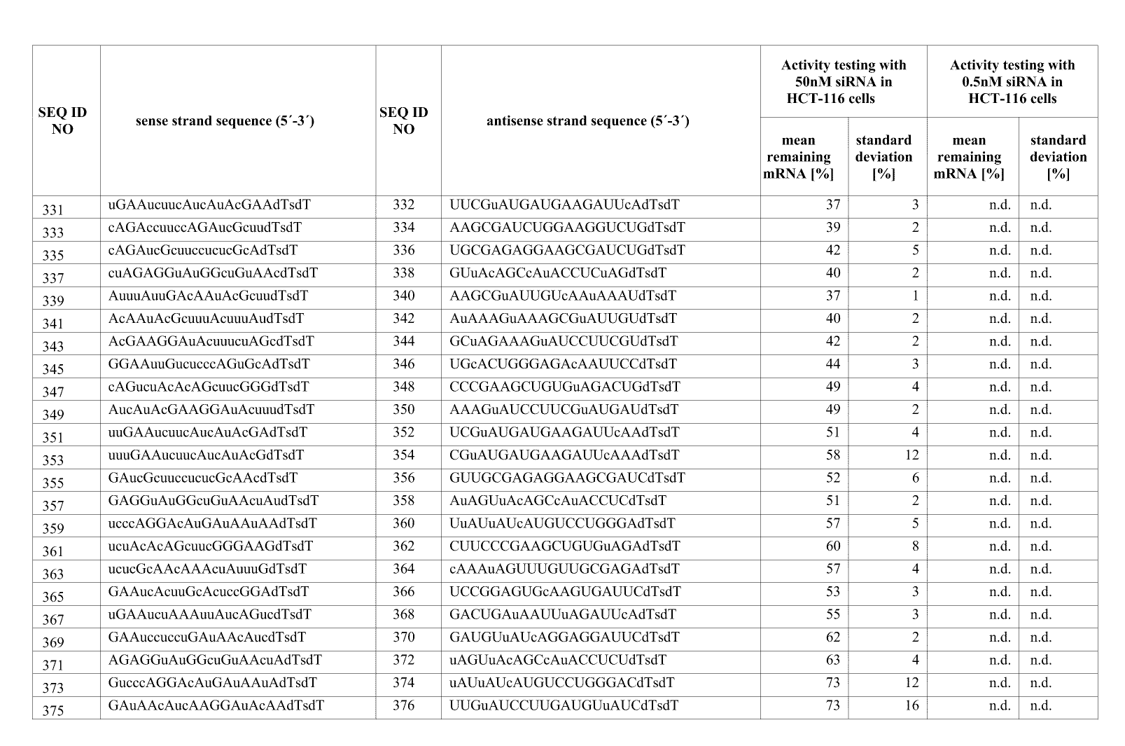

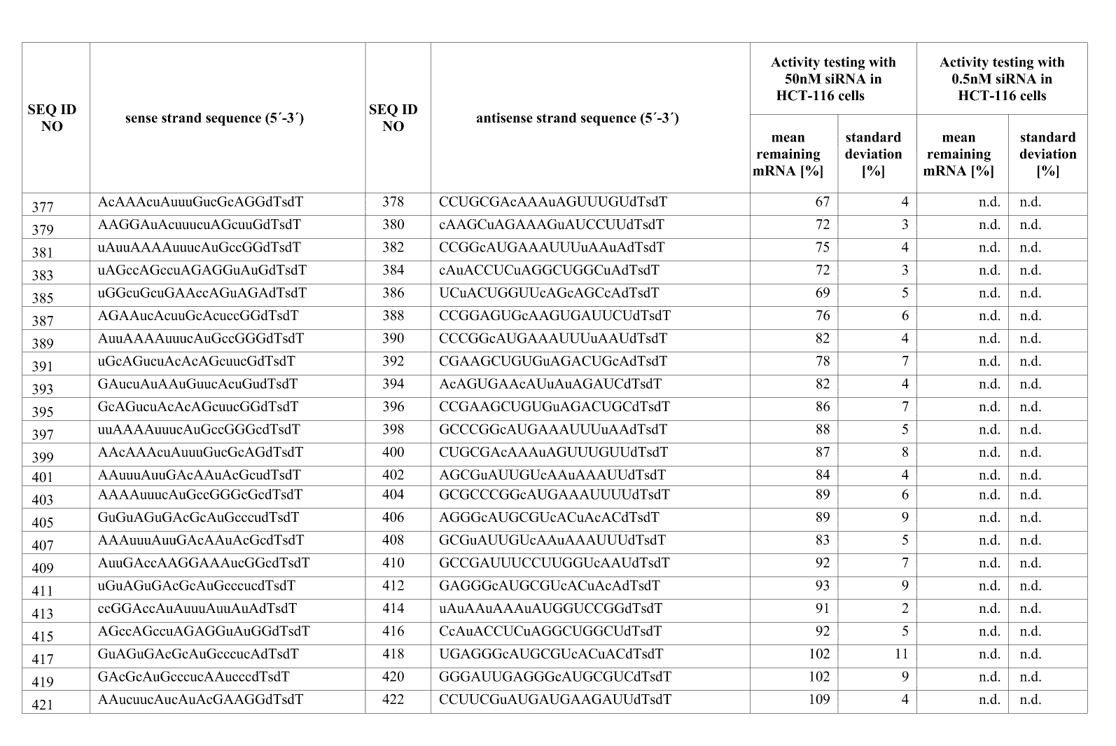

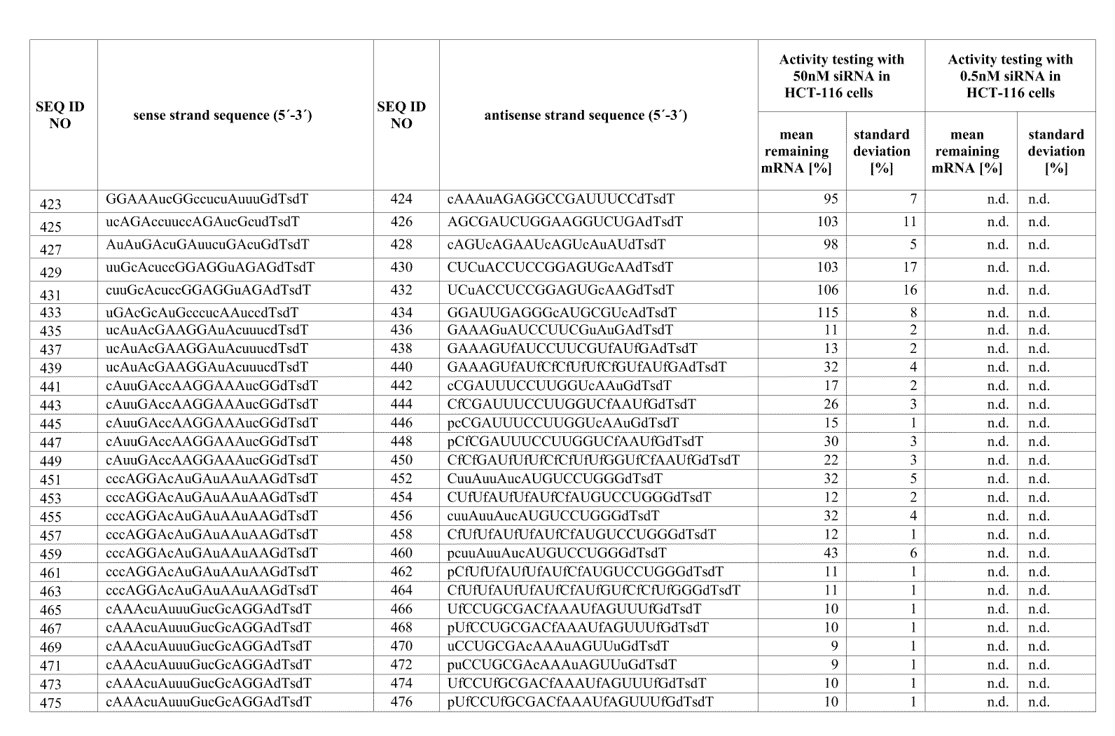

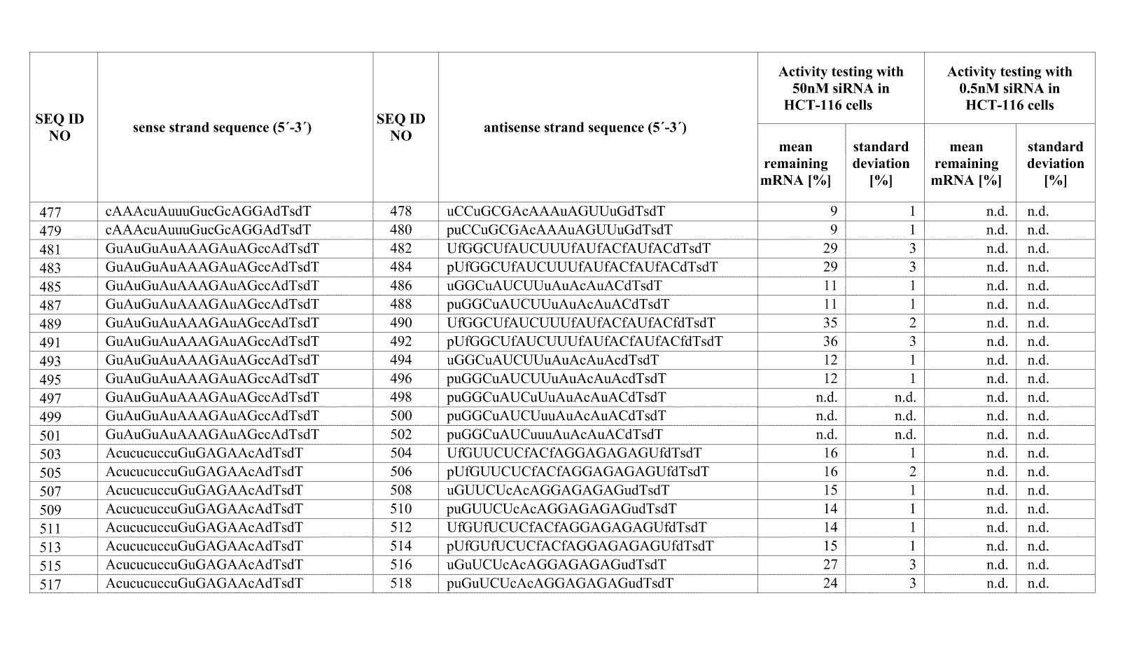

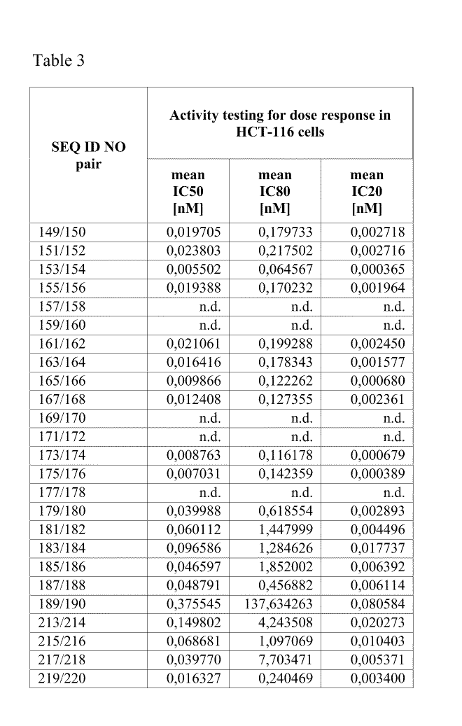

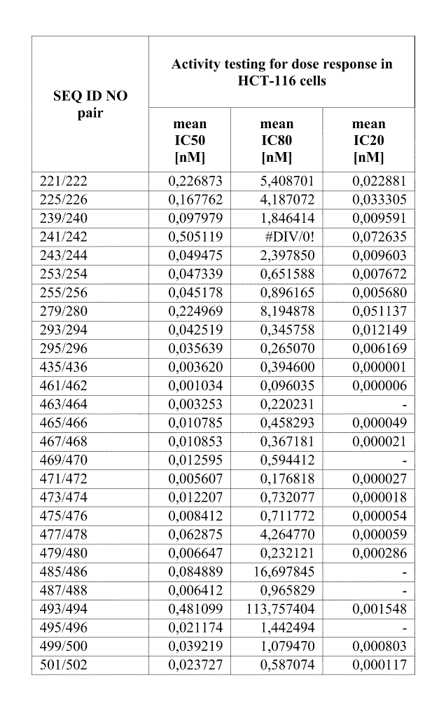

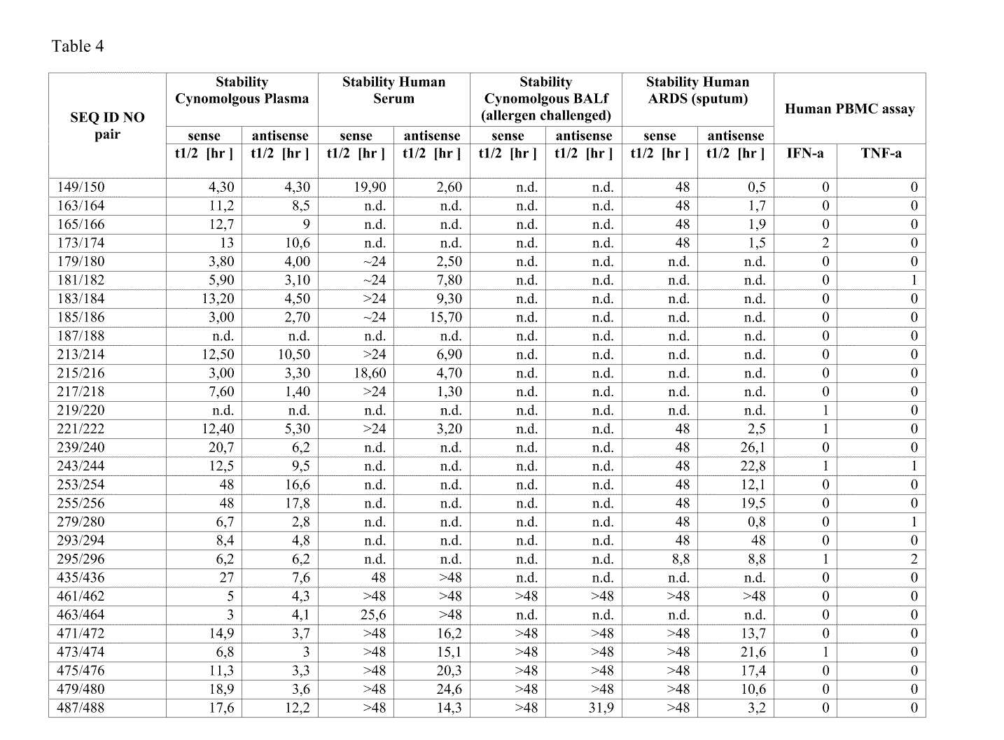

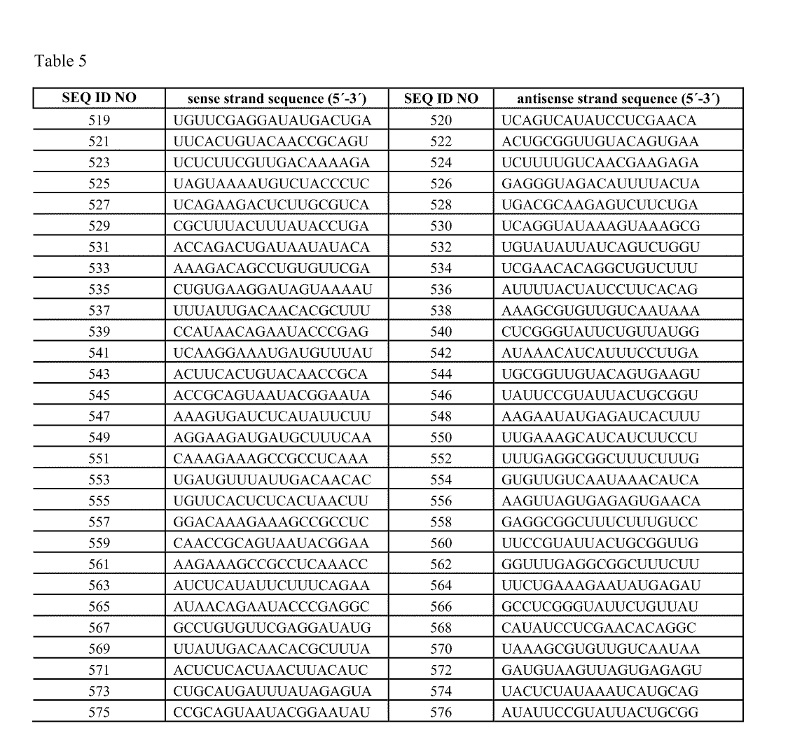

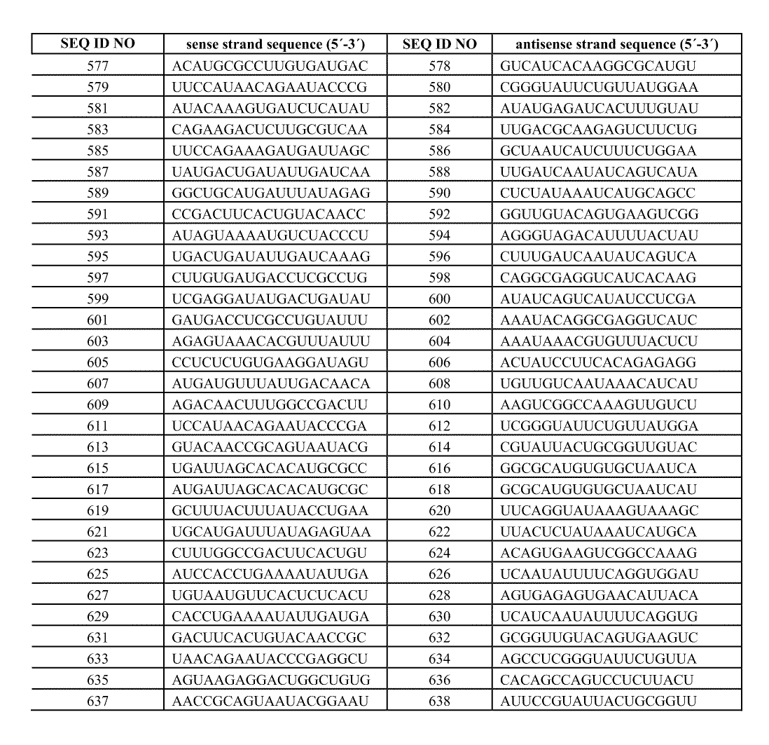

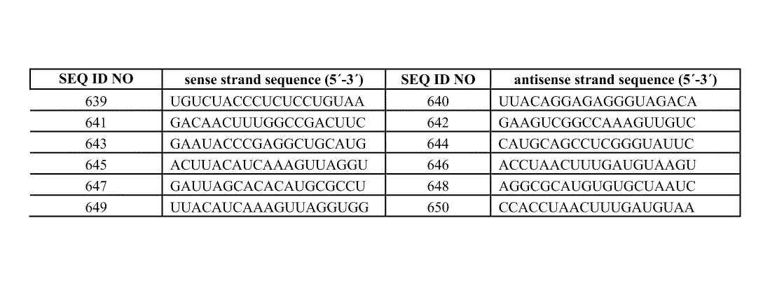

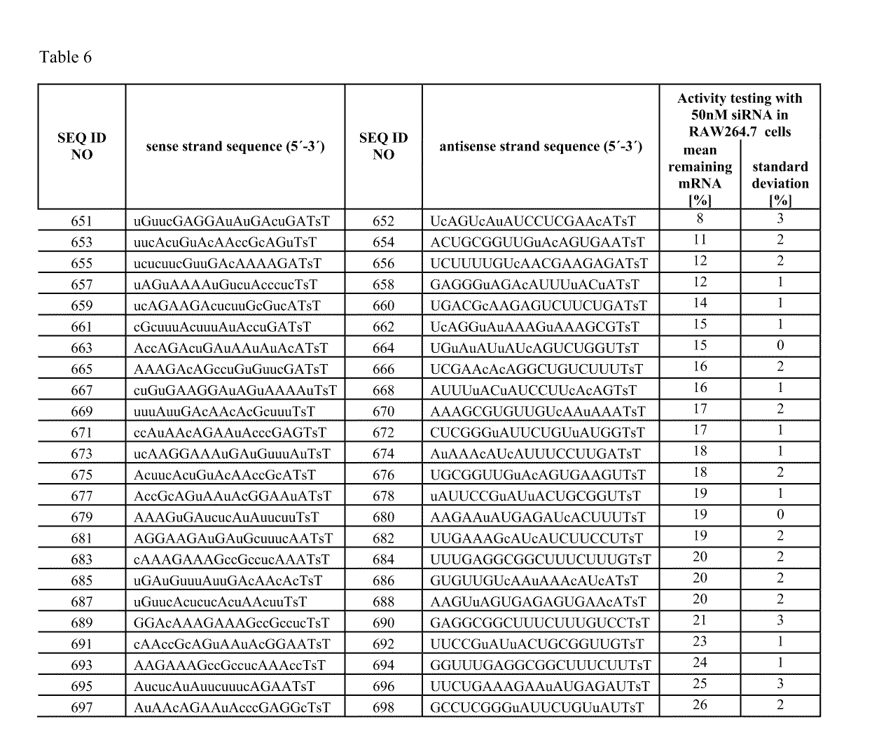

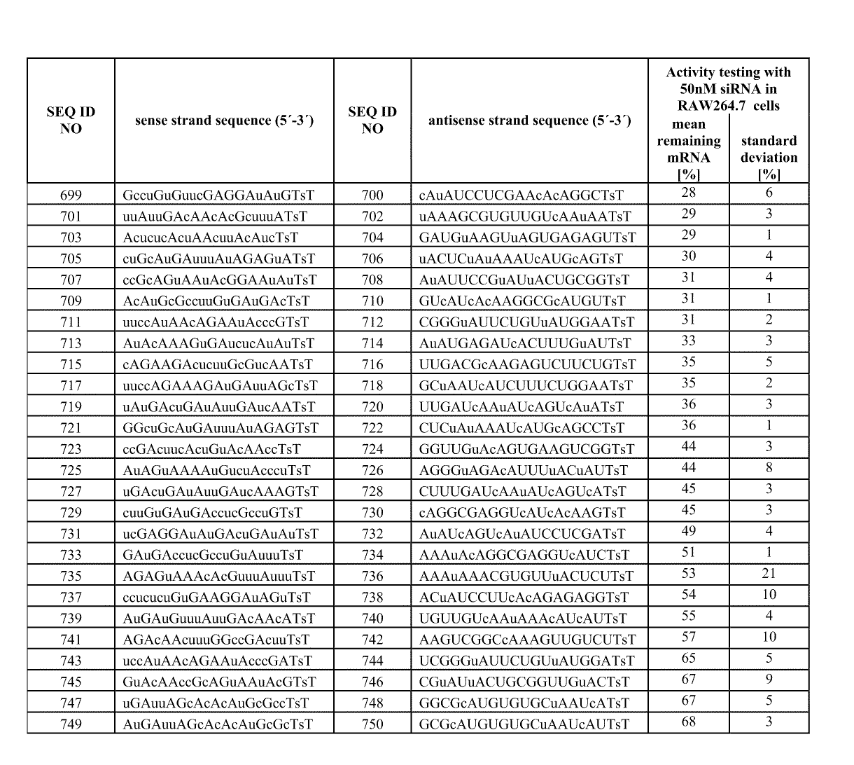

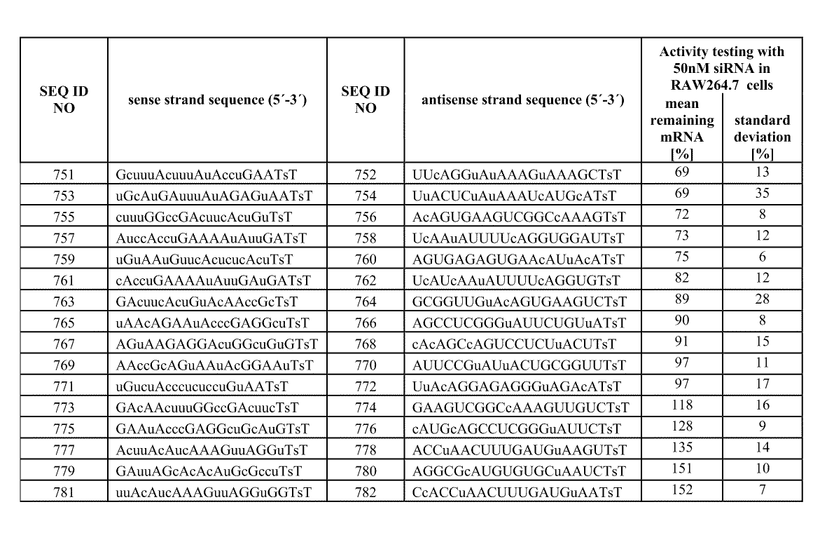

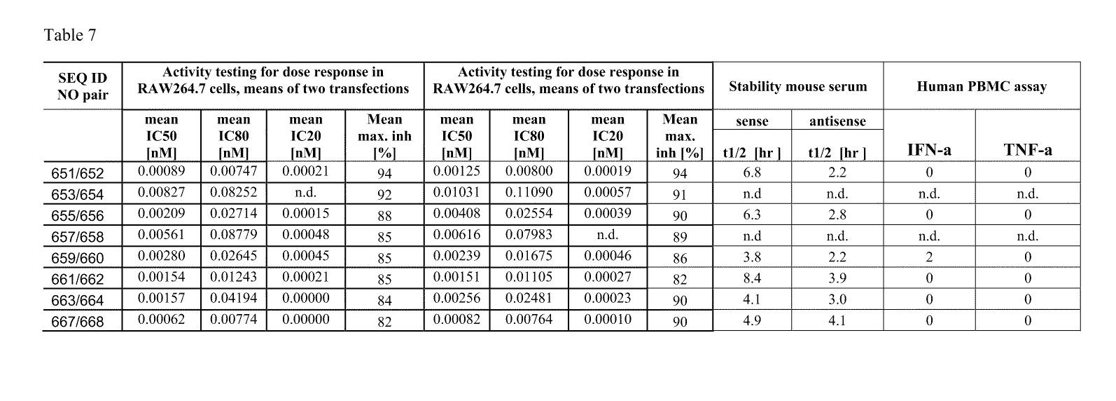

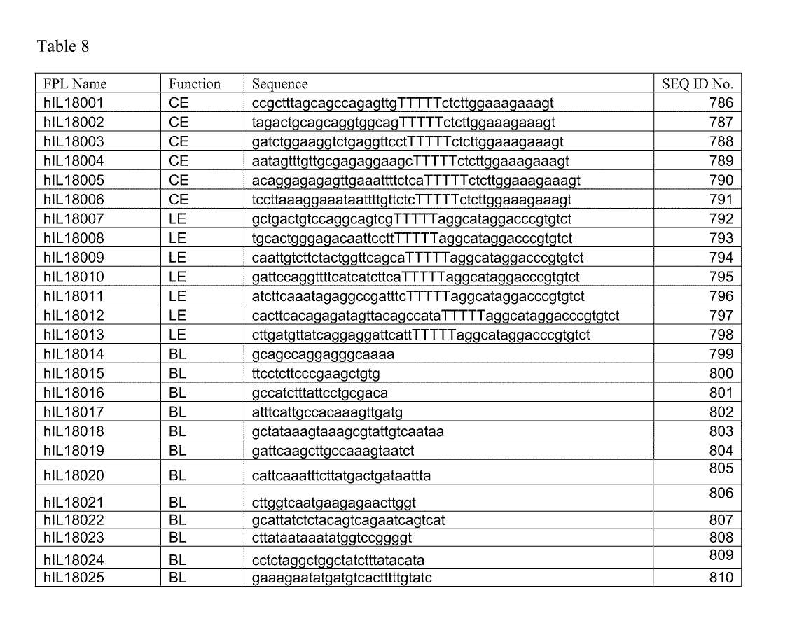

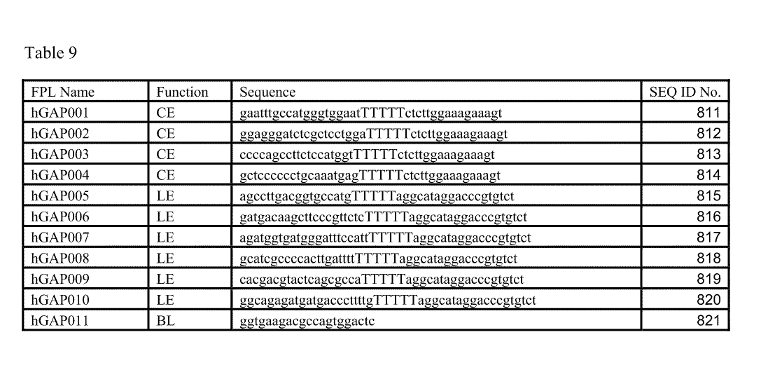

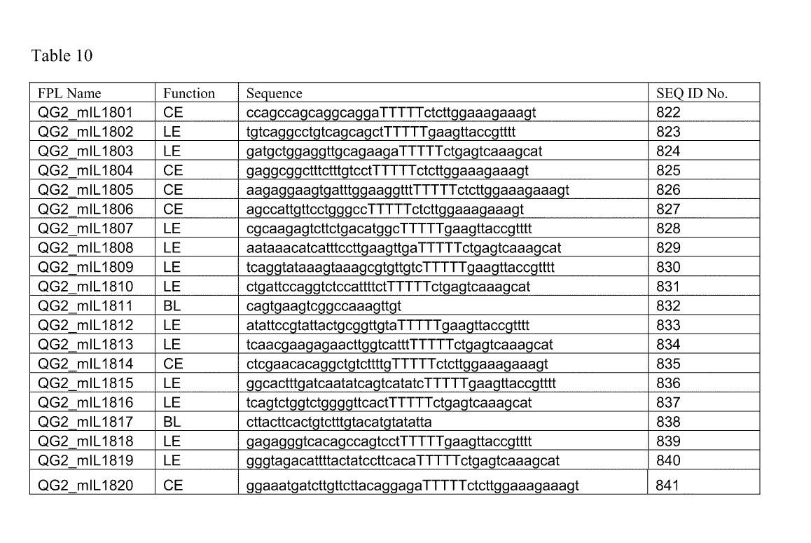

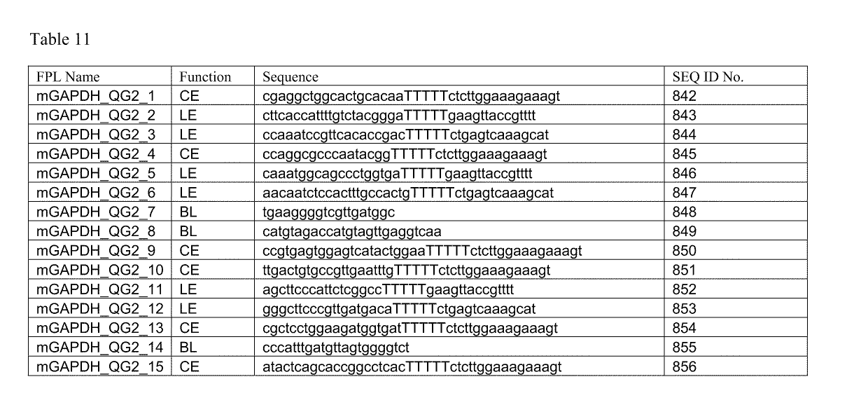

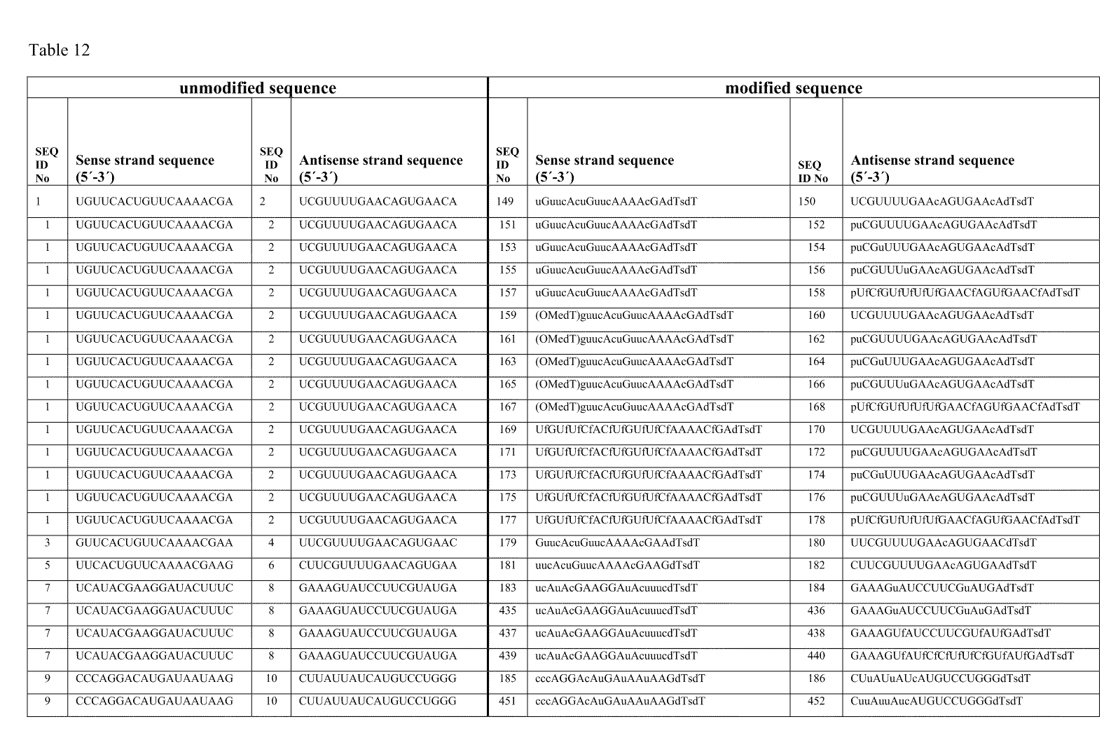

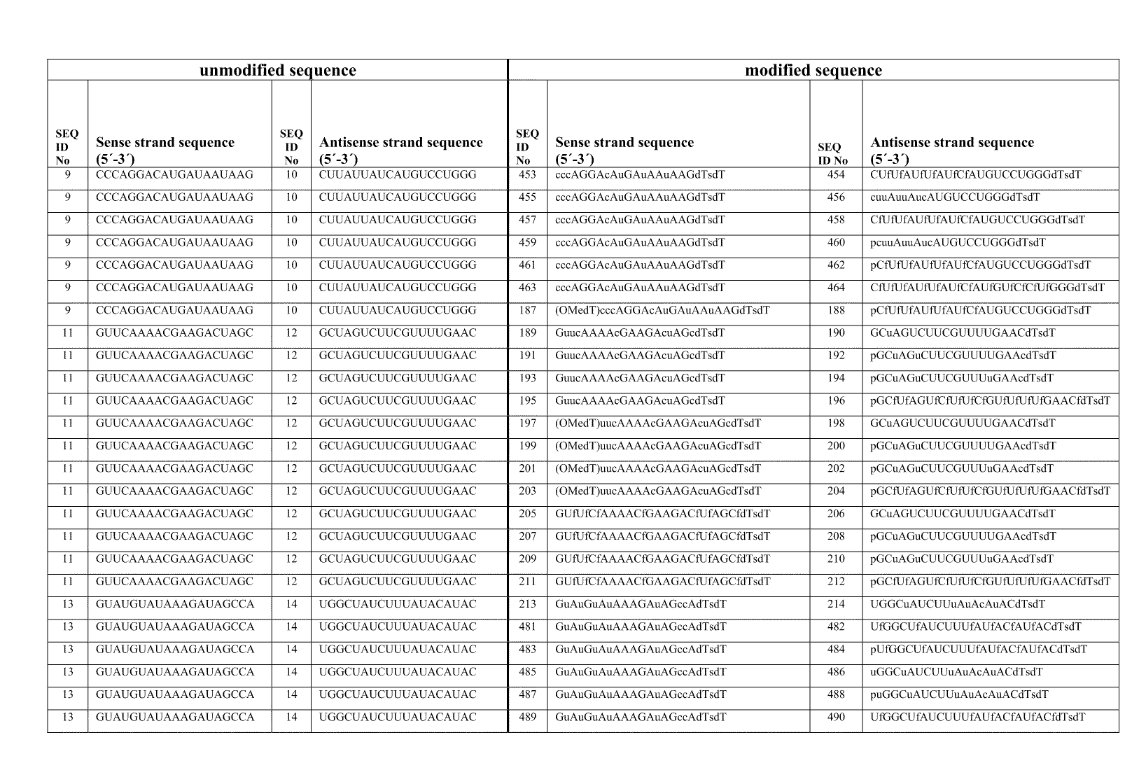

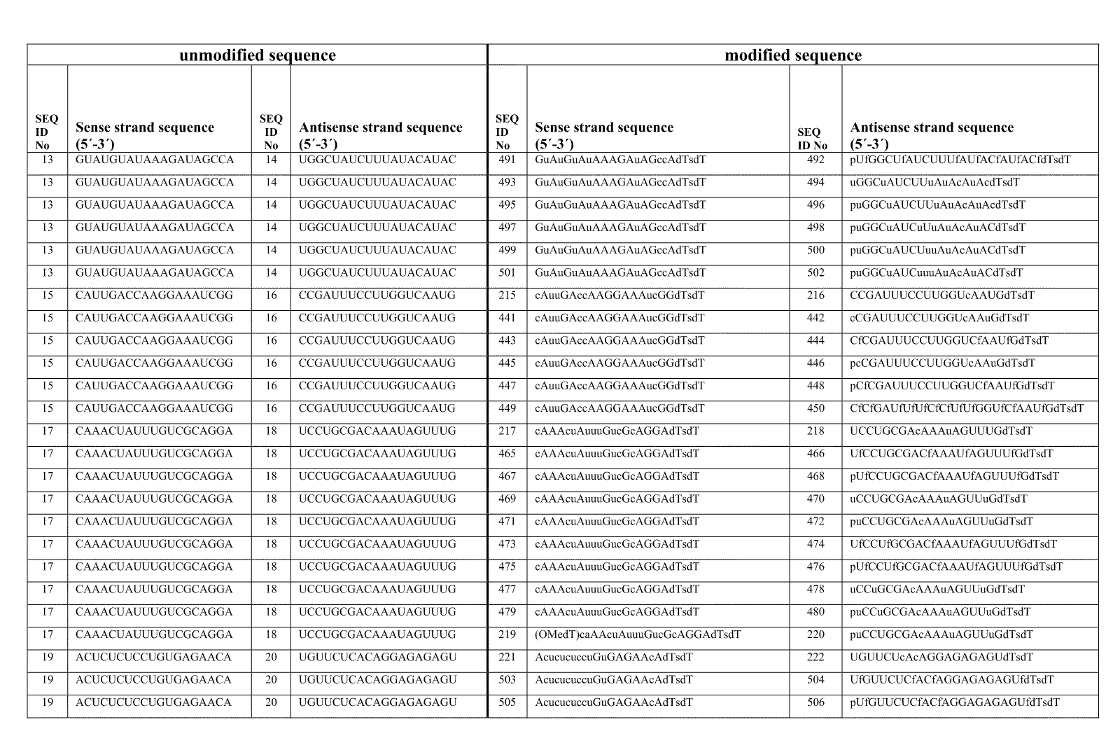

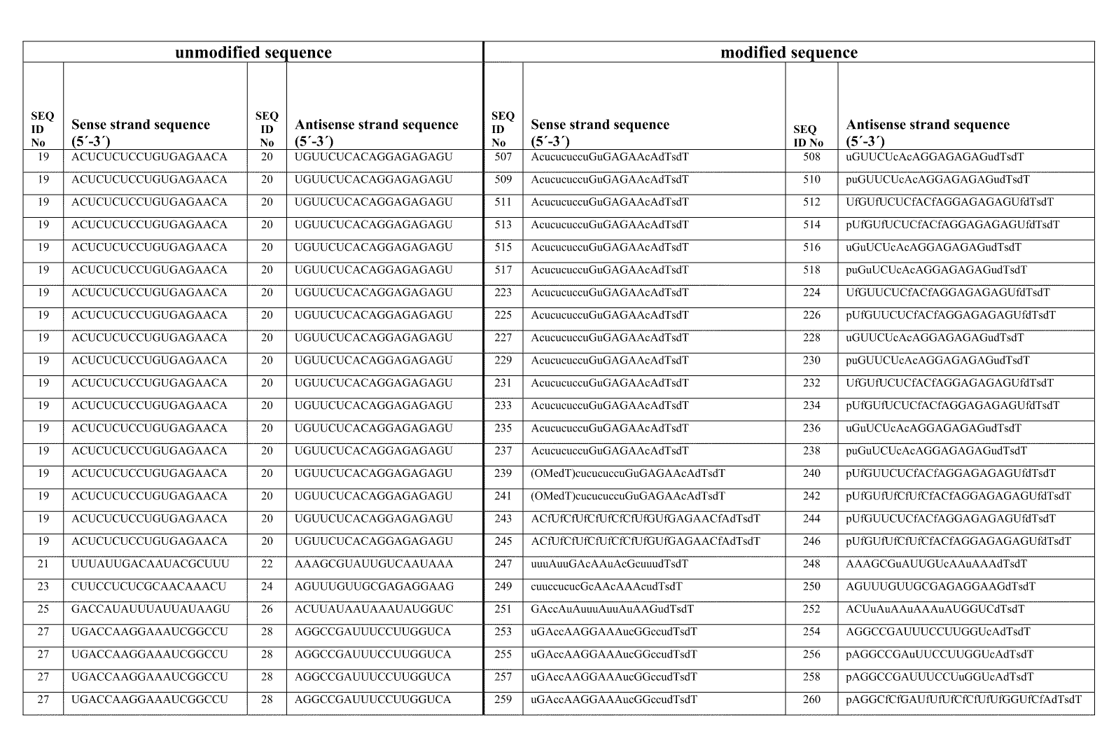

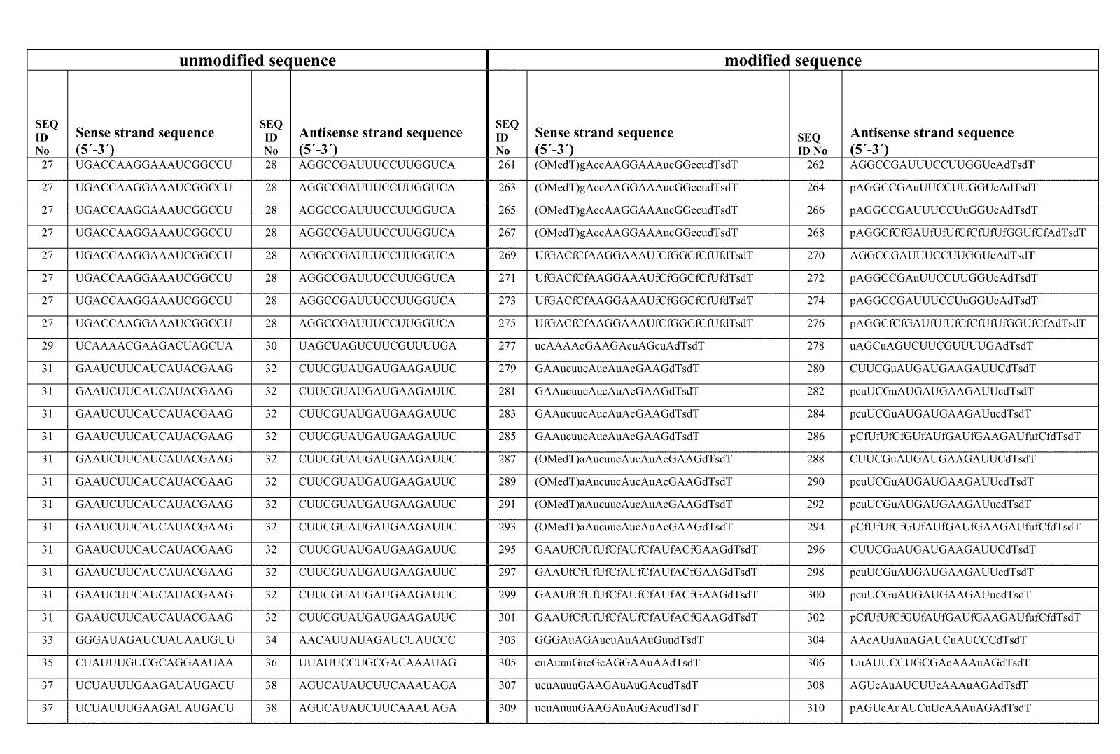

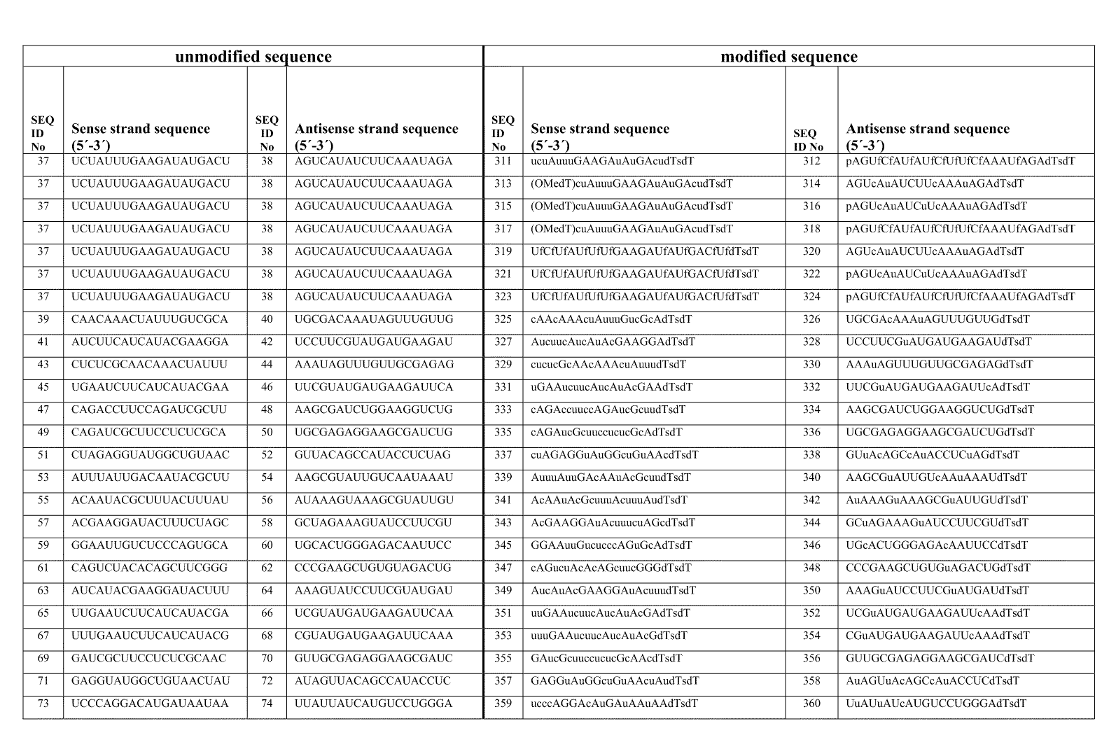

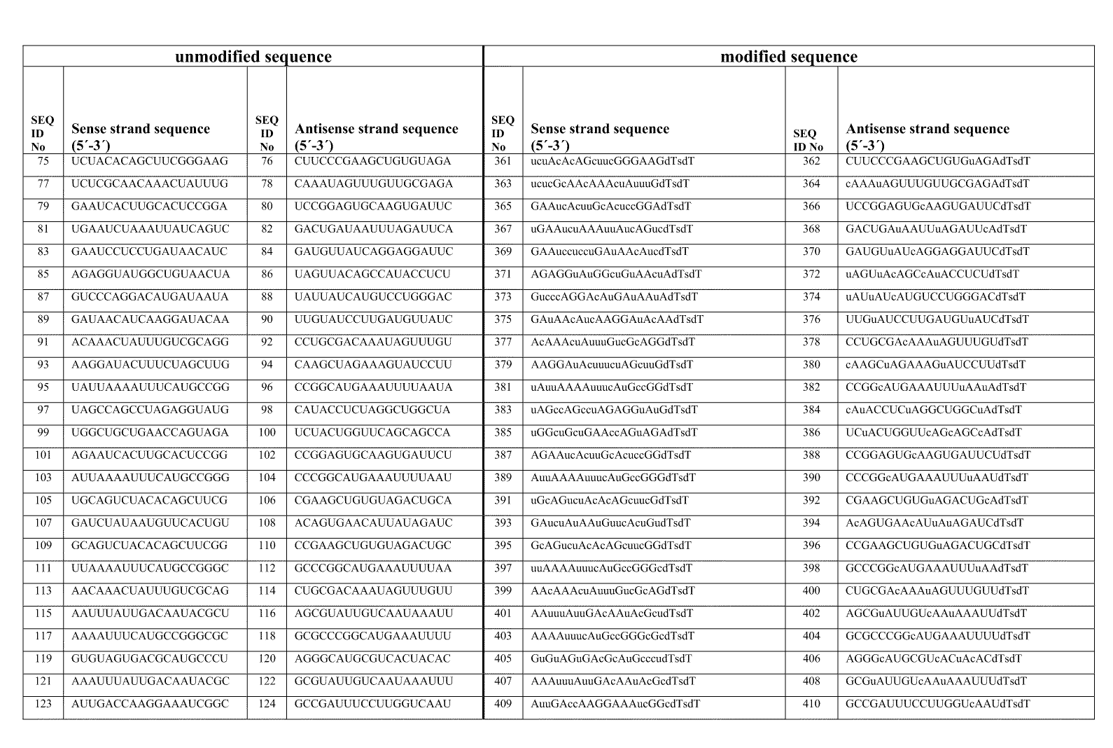

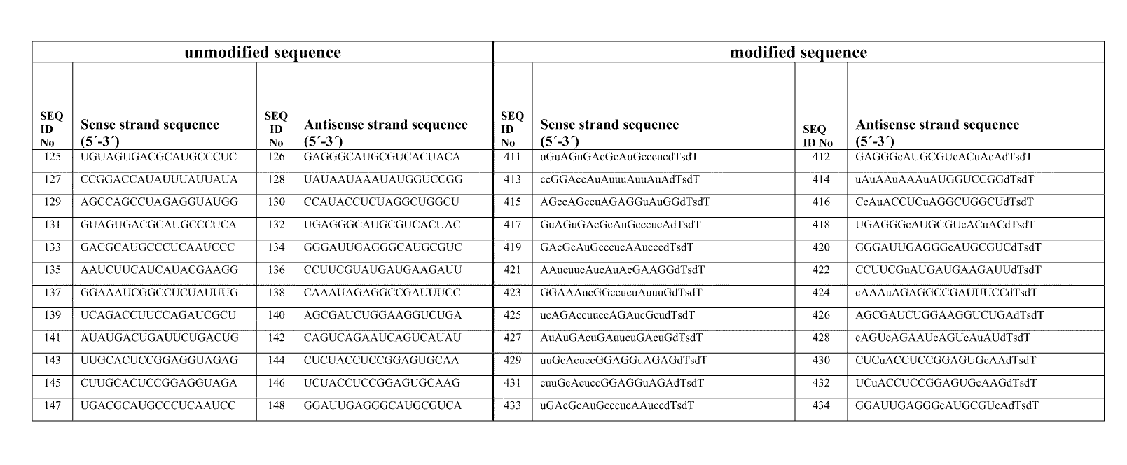

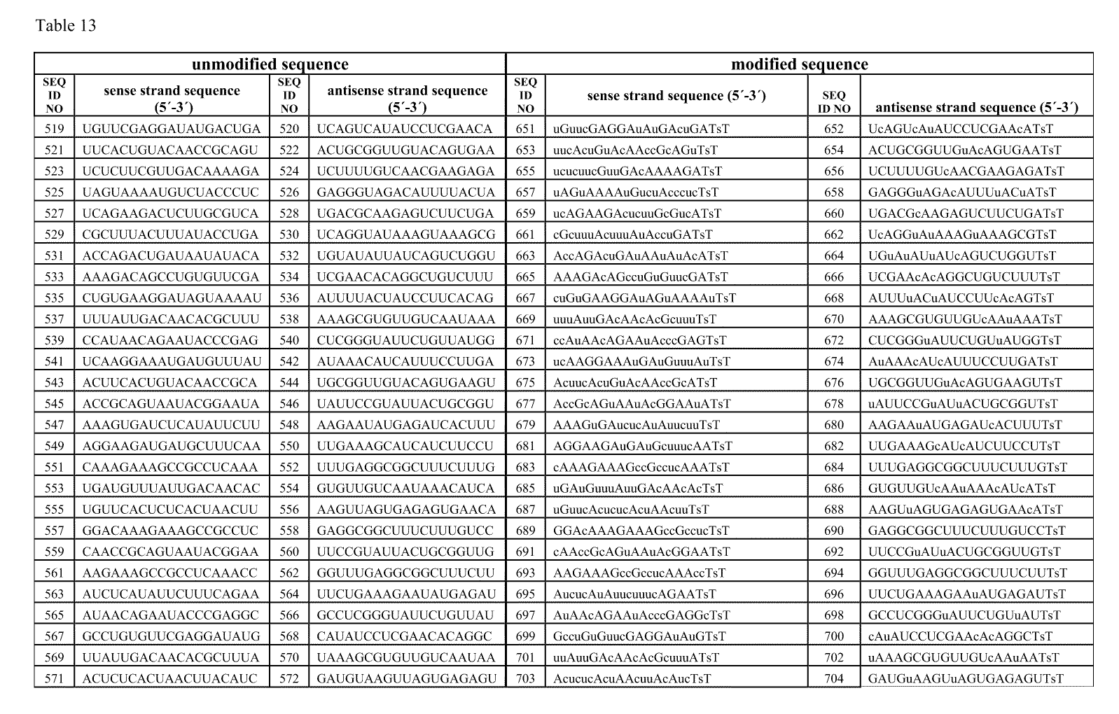

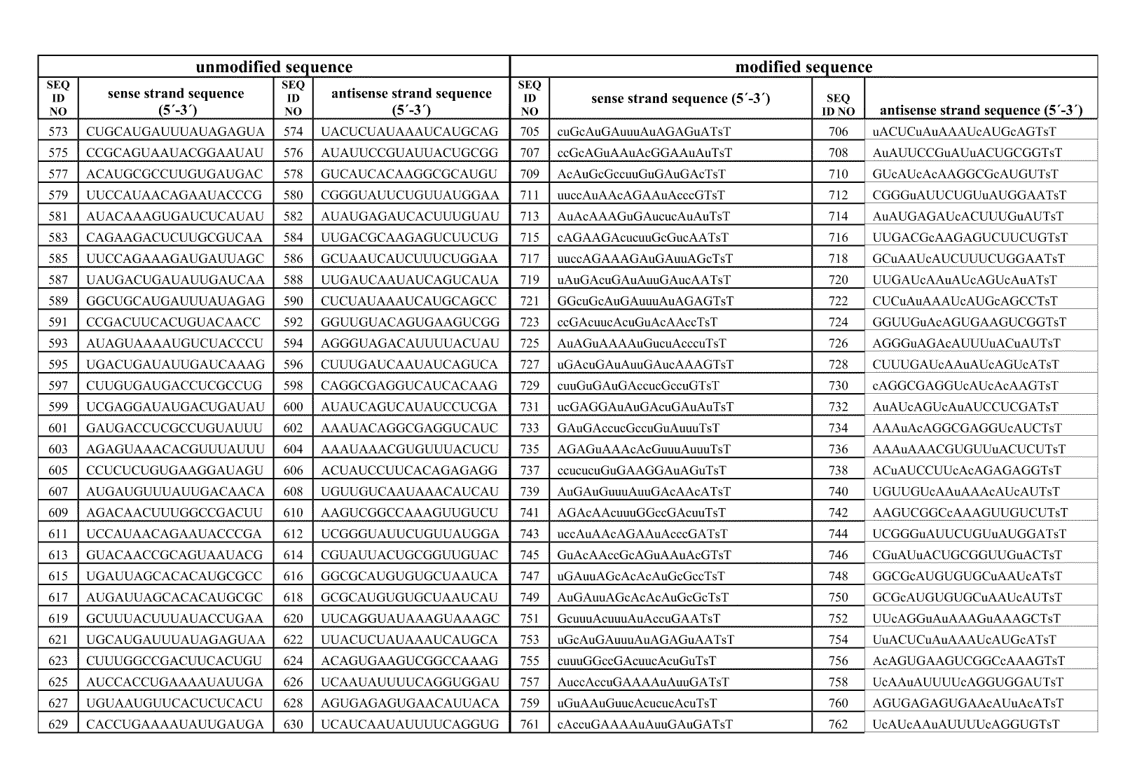

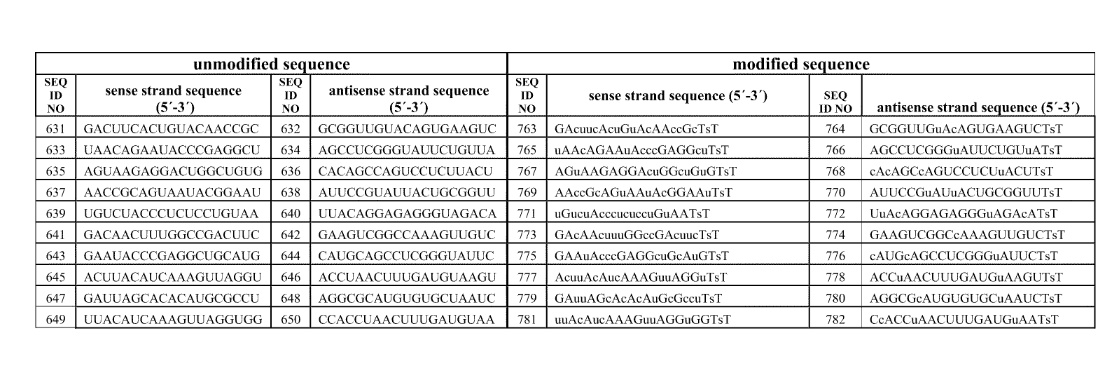

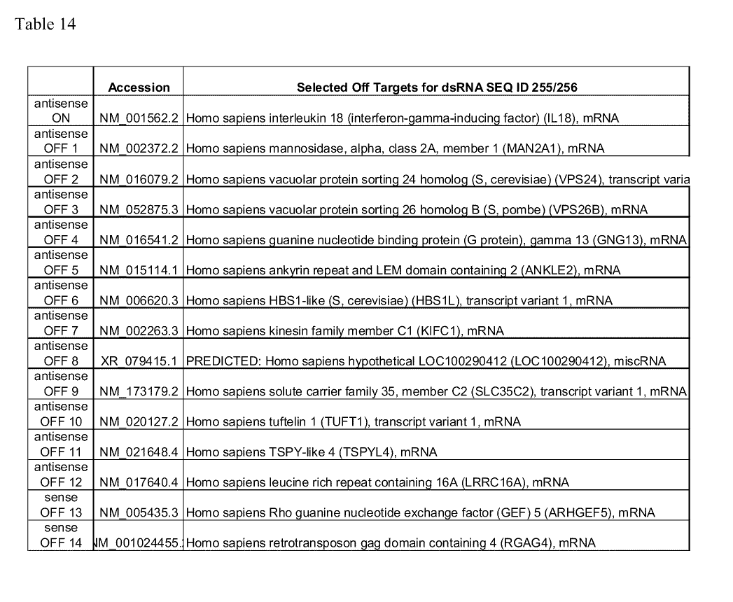

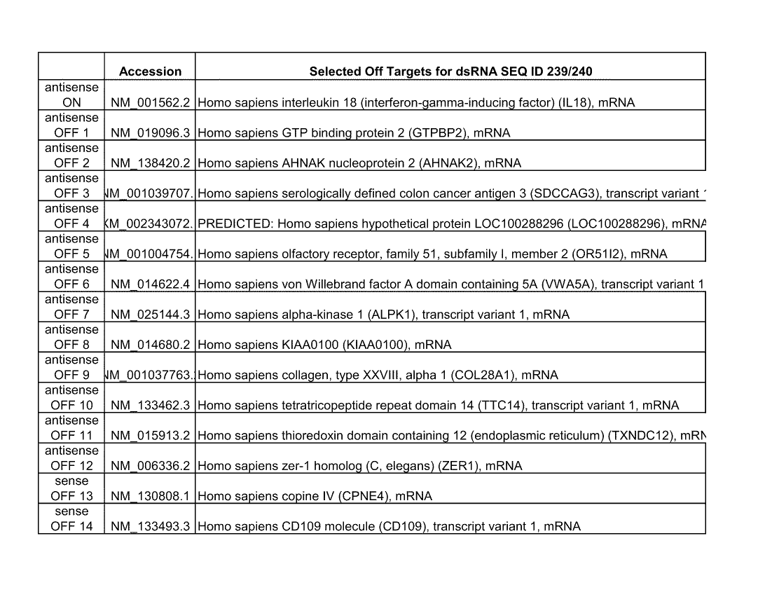

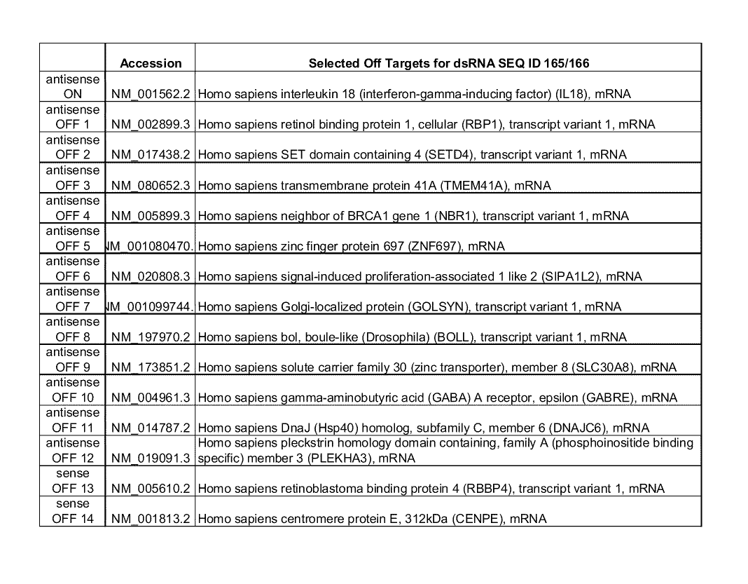

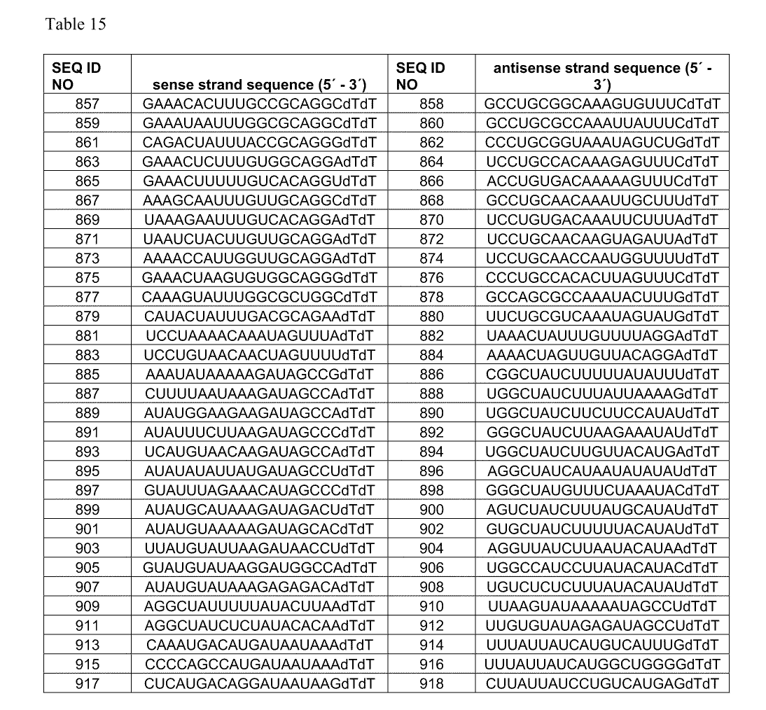

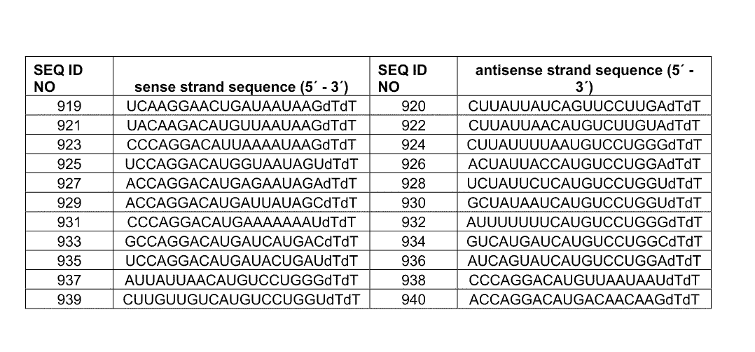

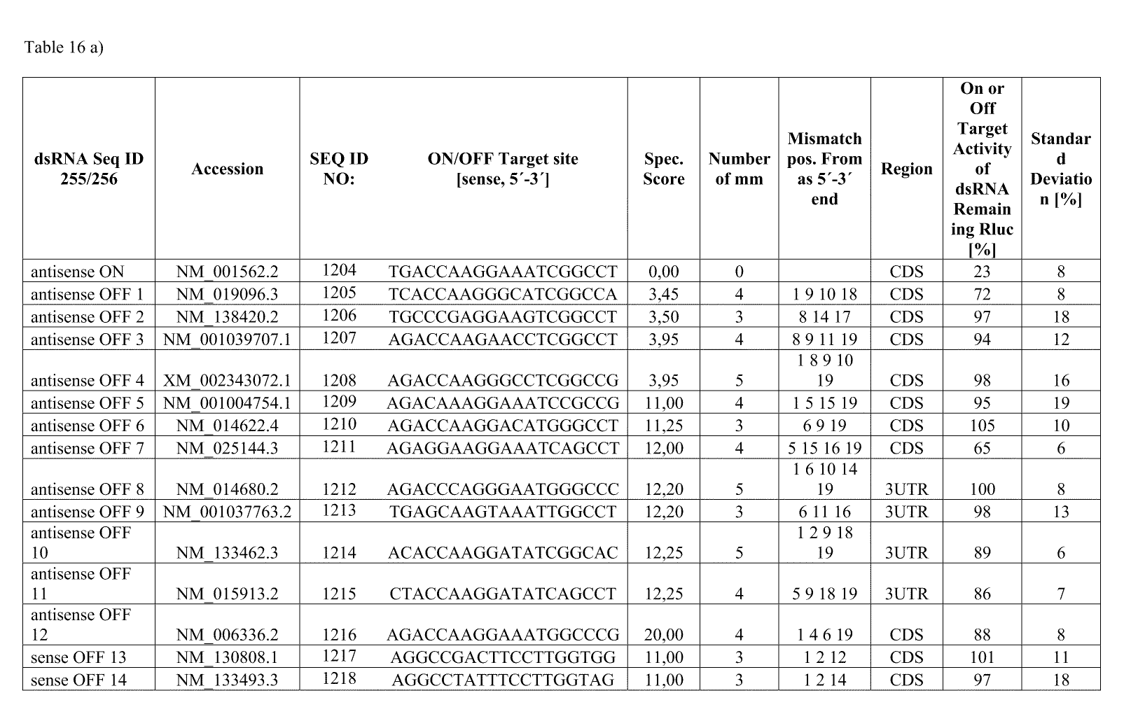

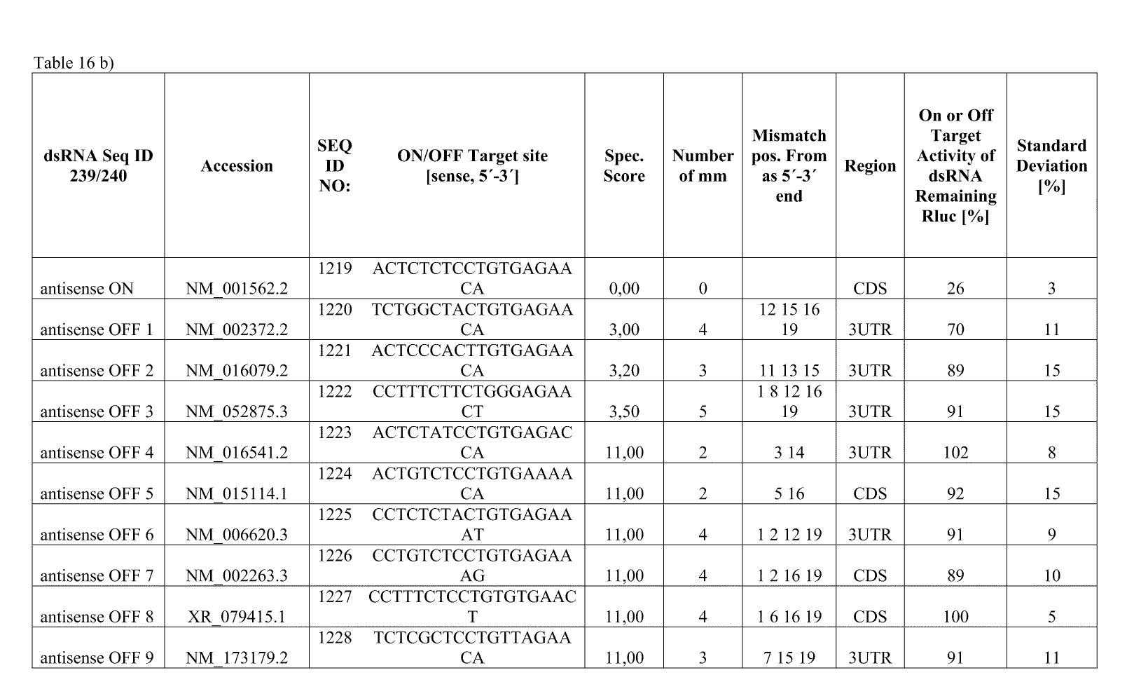

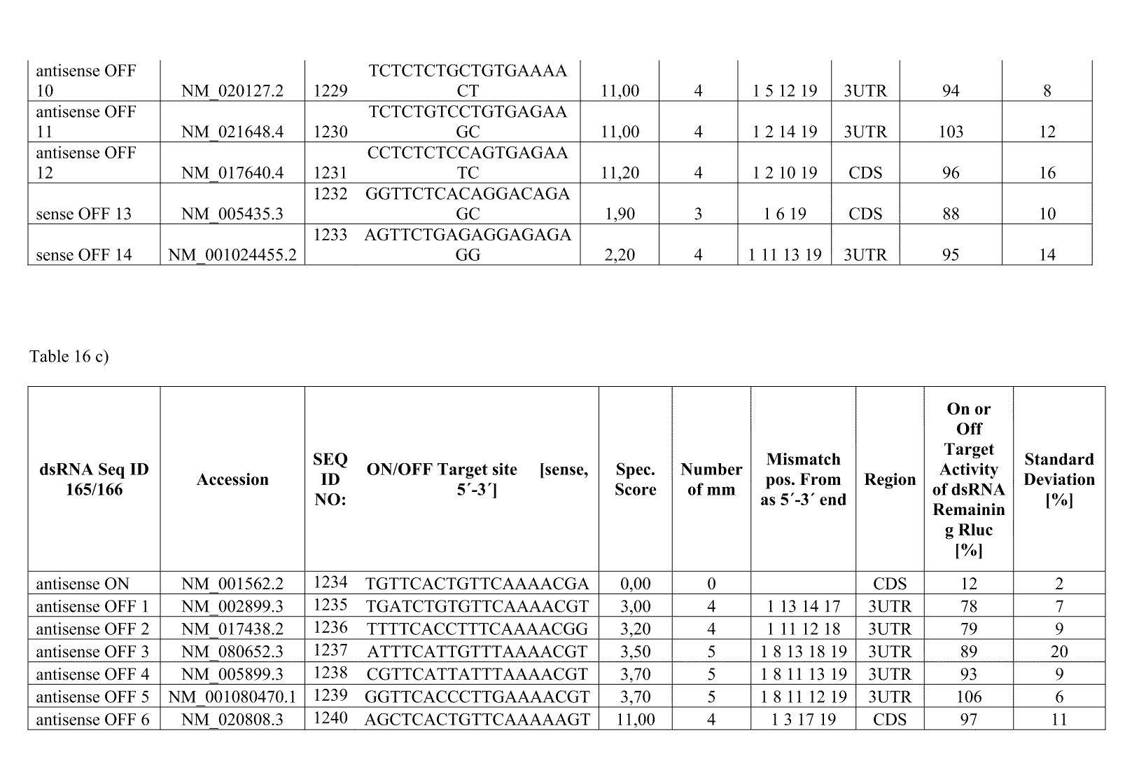

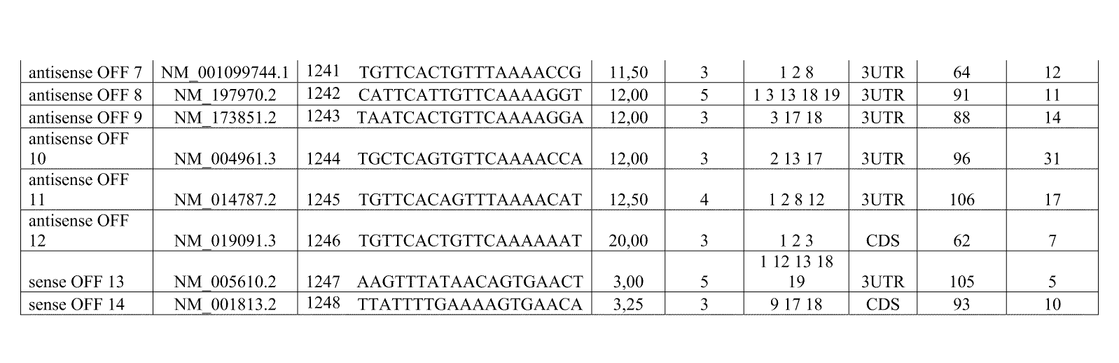

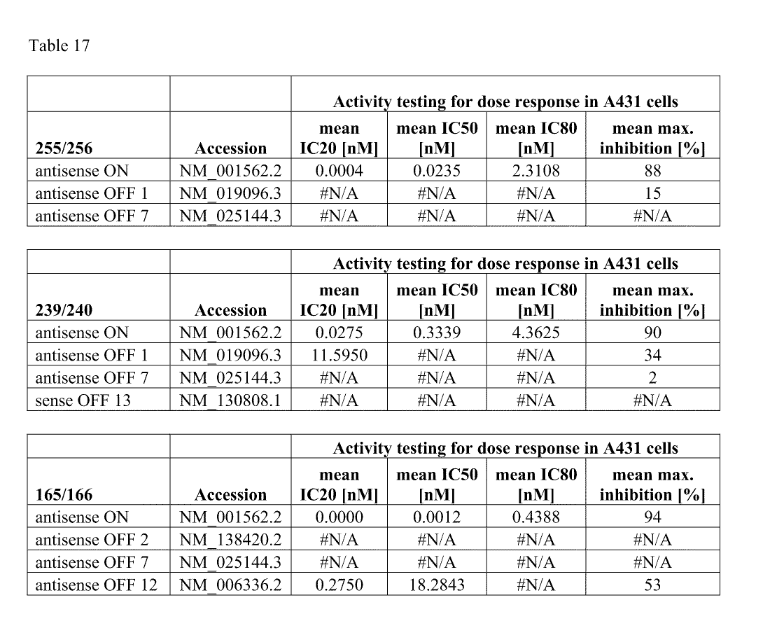

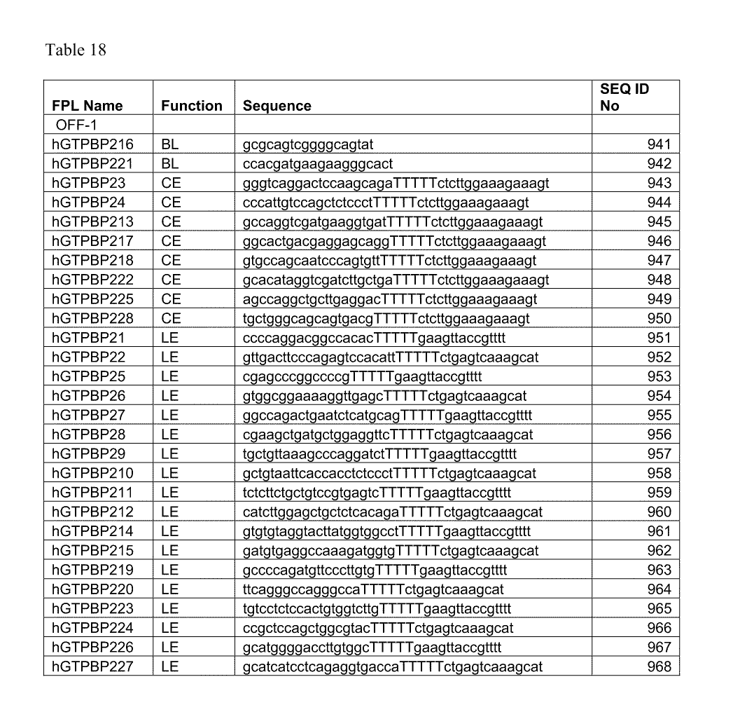

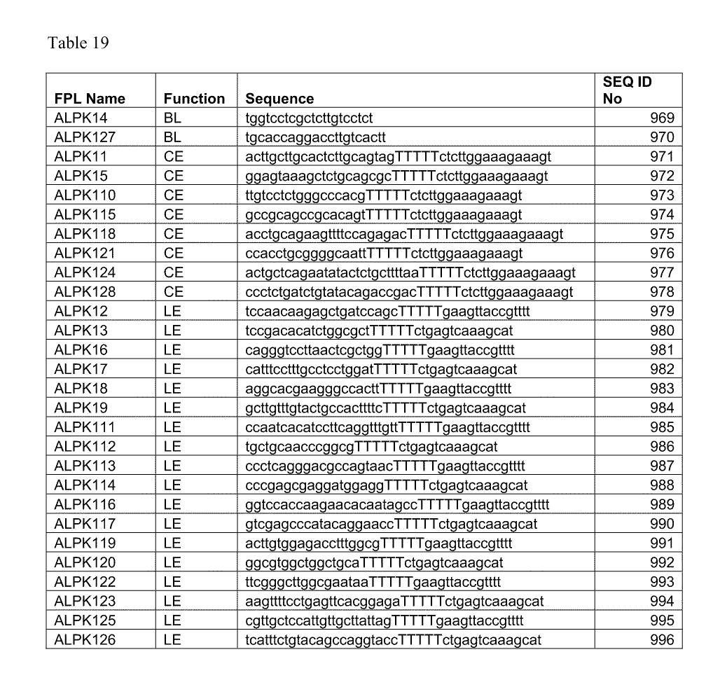

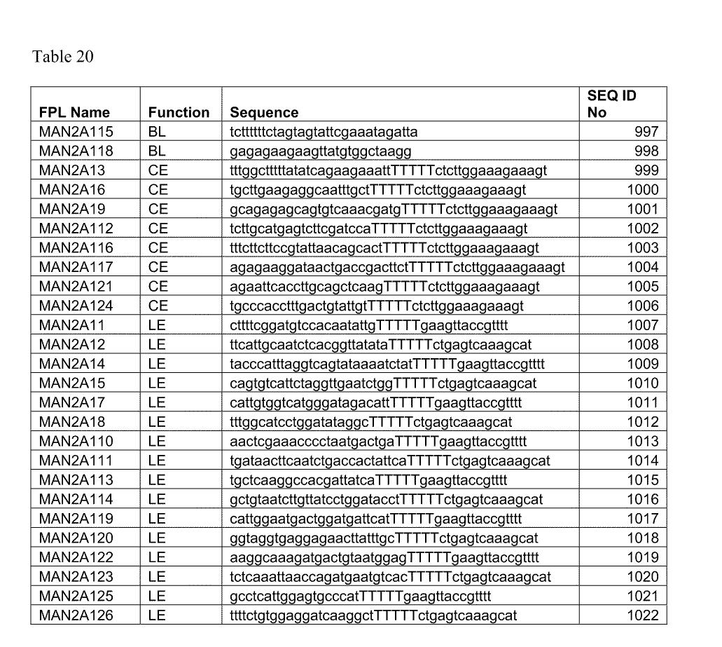

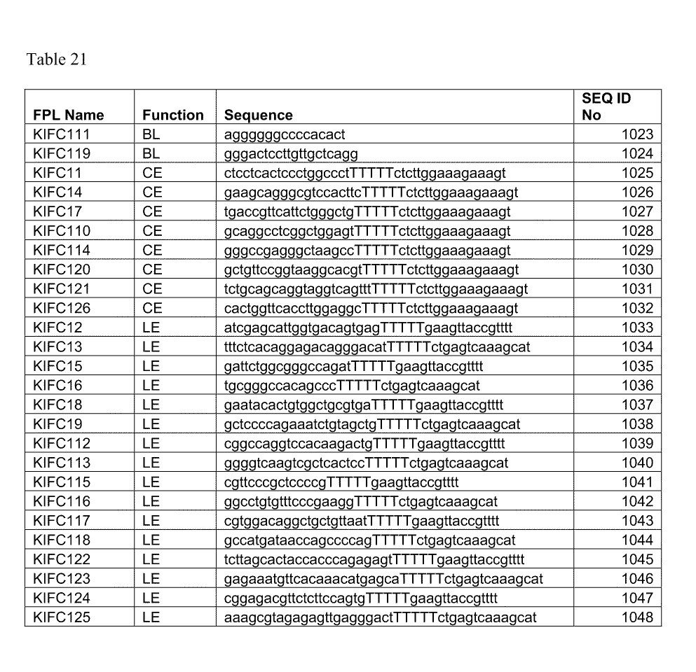

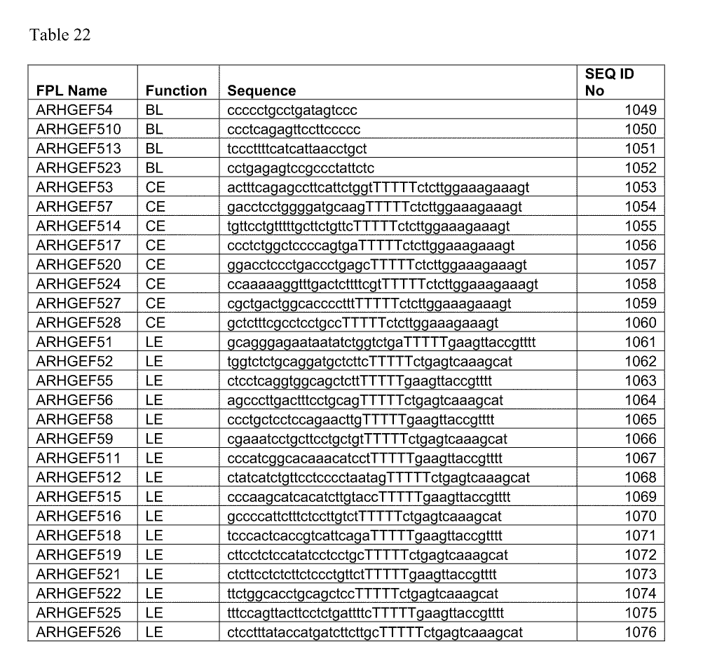

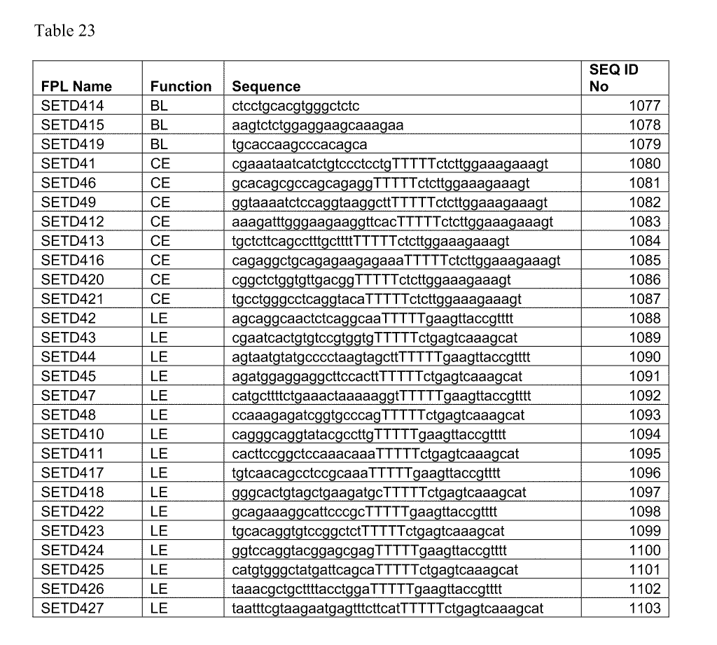

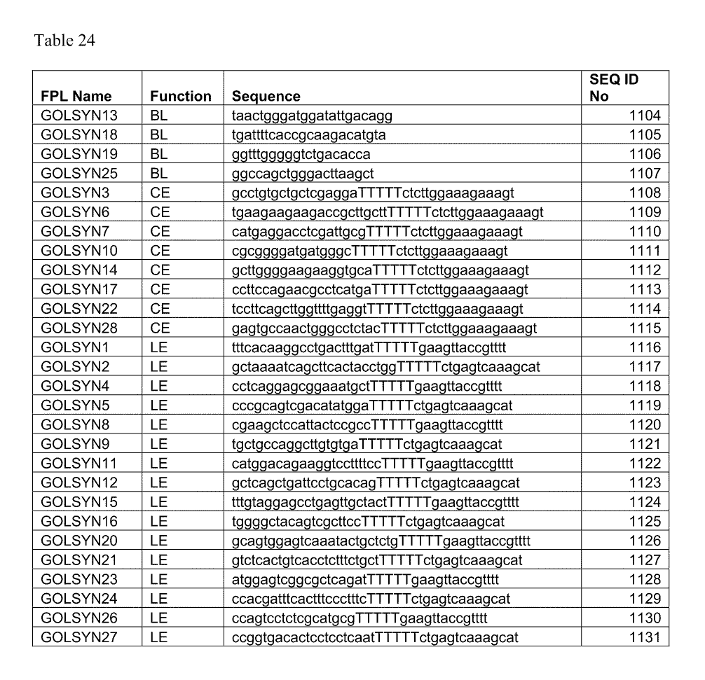

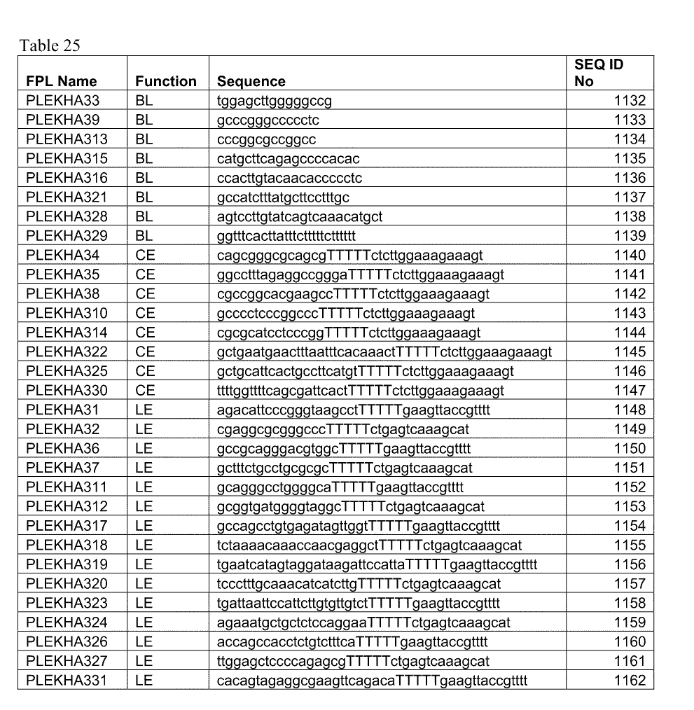

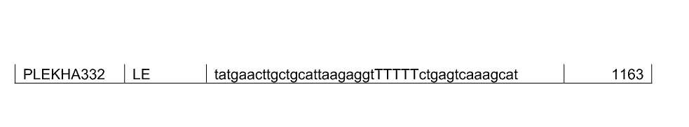

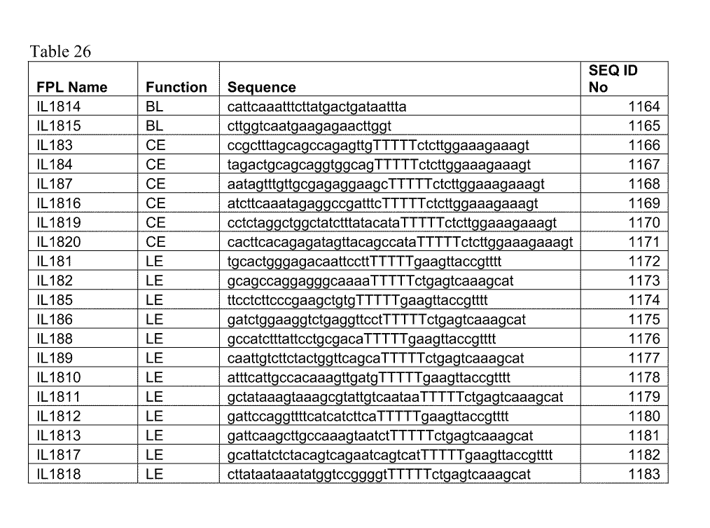

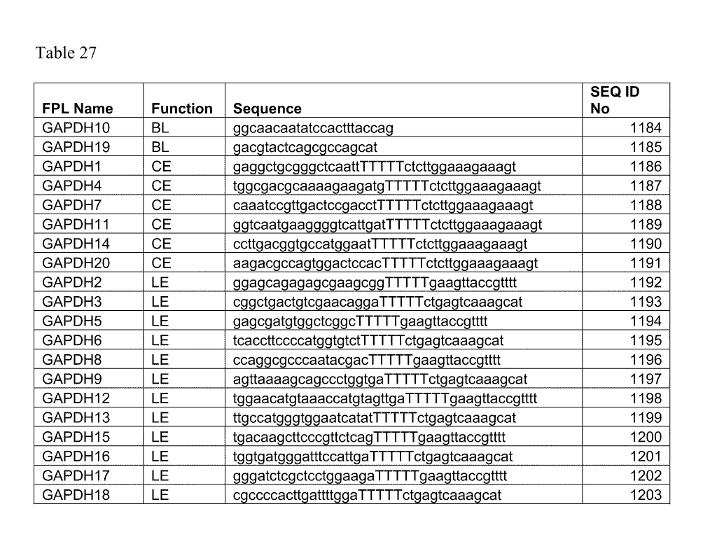

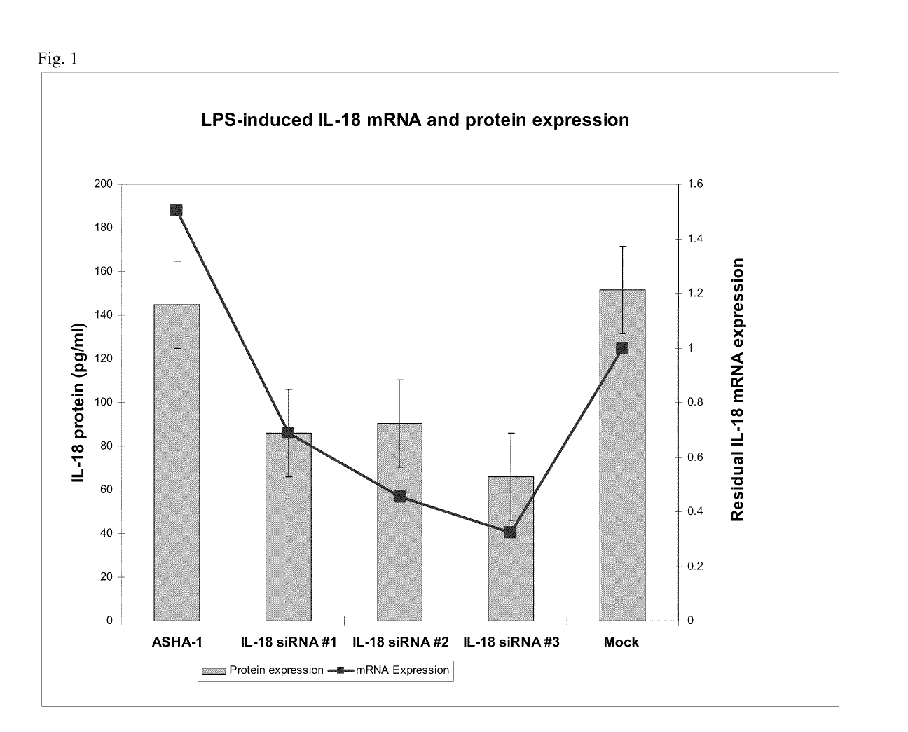

The level of IL-18 mRNA in siRNA-treated cells was calculated as residual IL-18 mRNA expression compared to mock transfection cells. The amount of IL-18 protein secreted from cells after LPS treatment was expressed in pg/ml. Results in the functional assay for IL-18 protein secretion and mRNA inhibition are given in appended FIG. 1.

IL-18 in vitro Analysis of Putative Off Targets

The psiCHECK™-vector (Promega) contains two reporter genes for monitoring RNAi-activity: a synthetic version of the Renilla luciferase (hRluc) gene and a synthetic firefly luciferase gene (hluc+). The firefly luciferase gene permits normalization of changes in Renilla luciferase expression to firefly luciferase expression. Renilla and firefly luciferase activities were measured using the Dual-Glo® Luciferase Assay System (Promega). To use the psiCHECK™ vectors for analyzing off-target effects of the inventive dsRNAs, the predicted off-target sequence was cloned into the multiple cloning region located 3′ to the synthetic Renilla luciferase gene and its translational stop codon. After cloning, the vector is transfected into a mammalian cell line, and subsequently cotransfected with dsRNAs targeting IL-18. If the dsRNA effectively initiates the RNAi process on the target RNA of the predicted off-target, the fused Renilla target gene mRNA sequence will be degraded, resulting in reduced Renilla luciferase activity.

### In Silico Off-Target Prediction

The human genome was searched by computer analysis for sequences homologous to the inventive dsRNAs. Homologous sequences that displayed less than 6 mismatches with the inventive dsRNAs were defined as a possible off-targets. Off-targets selected for in vitro off target analysis are given in appended table 14.

Generation of psiCHECK Vectors Containing Predicted Off-Target Sequences

The strategy for analyzing potential off-target effects for an siRNA lead candidate includes the cloning of the predicted off-target sites into the psiCHECK2 Vector system (Dual Glo®-system, Promega, Braunschweig, Germany cat. No C8021) via XhoI and NotI restriction sites. Therefore the off-target site is extended with 10 nucleotides upstream and downstream of the dsRNA target site followed by the sequence for cloning. Additionally a NheI restriction site is integrated to prove insertion of the fragment by restriction analysis. The single-stranded oligonucleotides were annealed according to a standard protocol (e.g. protocol by Metabion) in a Mastercycler (Eppendorf) and then cloned into psiCHECK (Promega) previously digested with XhoI and NotI restriction enzymes (e.g. New England Biolabs). Successful insertion was verified by restriction analysis with NheI and subsequent sequencing of the positive clones. After clonal production the correct plasmids were used in cell culture experiments.

Analysis of dsRNA Off-Target Effects

Cell Culture: Cos7 cells were obtained from Deutsche Sammlung für Mikroorganismen and Zellkulturen (DSMZ, Braunschweig, Germany, cat. No. ACC-60) and cultured in DMEM (Biochrom AG, Berlin, Germany, cat. No. F0435) supplemented to contain 10% fetal calf serum (FCS) (Biochrom AG, Berlin, Germany, cat. No. S0115), Penicillin 100 U/ml, and Streptomycin 100 μg/ml (Biochrom AG, Berlin, Germany, cat. No. A2213) and 2 mM L-Glutamine (Biochrom AG, Berlin, Germany, cat. No. K0283) as well as 12 μg/ml Natrium-bicarbonate at 37° C. in an atmosphere with 5% CO2 in a humidified incubator (Heraeus HERAcell, Kendro Laboratory Products, Langenselbold, Germany).

Transfection and Luciferase quantification: For transfection with plasmids, Cos-7 cells were seeded at a density of 2.25×104 cells/well in 96-well plates and transfected directly. Transfection of plasmids was carried out with lipofectamine 2000 (Invitrogen GmbH, Karlsruhe, Germany, cat. No. 11668-019) as described by the manufacturer at a concentration of 50 ng/well. 4 h after transfection, the medium was discarded and fresh medium was added. Now the siRNAs were transfected in a concentration at 50 nM using lipofectamine 2000 as described above. 24 h after siRNA transfection the cells were lysed using Luciferase reagent described by the manufacturer (Dual-Glo™ Luciferase Assay system, Promega, Mannheim, Germany, cat. No. E2980) and Firefly and Renilla Luciferase were quantified according to the manufacturer's protocol. Renilla Luciferase protein levels were normalized to Firefly Luciferase levels. For each siRNA eight individual data points were collected in two independent experiments. A siRNA unrelated to all target sites was used as a control to determine the relative Renilla Luciferase protein levels in siRNA treated cells. All results of the transfection experiments are summarized in table 16.

Endogenous analysis was performed with off targets showing a Renilla Luciferase knockdown of more than 25% from single dose screen at 50 nM. Those were further characterized in dose response curves in concentrations ranging from 100 nM down to 10 fM in 6-fold dilutions. The transfection was performed as described above using Lipofectamine 2000 in human A431 cells. A431 cells were obtained from Deutsche Sammlung für Mikroorganismen and Zellkulturen (DSMZ, Braunschweig, Germany, cat. No. ACC-91) and cultured in RPMI (Biochrom AG, Berlin, Germany, cat. No. FG 1215) supplemented to contain 10% fetal calf serum (FCS) (Biochrom AG, Berlin, Germany, cat. No. S0115), Penicillin 100 U/ml, and Streptomycin 100 μg/ml (Biochrom AG, Berlin, Germany, cat. No. A2213) at 37° C. in an atmosphere with 5% CO2 in a humidified incubator (Heraeus HERAcell, Kendro Laboratory Products, Langenselbold, Germany). After transfection cells were incubated for 24 h at 37° C. and 5% CO2 in a humidified incubator (Heraeus GmbH, Hanau, Germany). For measurement of IL-18 mRNA and all off target mRNAs as well as GAPDH mRNA the QuantiGene 2.0 Assay Kit (Panomics, Fremont, Calif., USA) for bDNA quantitation of mRNA was used. Transfected A431 cells were harvested and lysed at 53° C. following procedures recommended by the manufacturer. 50 μl of the lysates were incubated with probesets specific for human IL-18 (table 26, SEQ ID No 1164-1183) or the specific off target mRNA (sequence of probesets see Table 18 to 25, SEQ ID No 941-1163) and processed according to the manufacturer's protocol for QuantiGene. For measurement of GAPDH mRNA 10 μl of the cell lysate was analyzed with the GAPDH specific probeset (table 27, SEQ ID No1184-1203). Chemoluminescence was measured in a Victor2-Light (Perkin Elmer, Wiesbaden, Germany) as RLUs (relative light units) and values obtained with the human IL-18 or off target probeset were normalized to the respective human GAPDH values for each well. Unrelated control siRNAs were used as a negative control. All data regarding the dose response experiments are given in appended table 17.

All ranges recited herein encompass all combinations and subcombinations included within that range limit. All patents and publications cited herein are hereby incorporated by reference in their entirety.

1. A double-stranded ribonucleic acid molecule capable of inhibiting the expression of the IL-18 gene in vitro by at least 60%.

2. The double-stranded ribonucleic acid molecule of claim 1, capable of inhibiting the expression of the IL-18 gene in vitro by at least 70%.

3. The double-stranded ribonucleic acid molecule of claim 1, capable of inhibiting the expression of the IL-18 gene in vitro by at least 80%.

4. The double-stranded ribonucleic acid molecule of claim 1, wherein said double-stranded ribonucleic acid molecule comprises a sense strand and an antisense strand, the antisense strand being at least partially complementary to the sense strand, whereby the sense strand comprises a sequence, which has an identity of at least 90% to at least a portion of an mRNA encoding IL-18, wherein said sequence is (i) located in the region of complementarity of said sense strand to said antisense strand; and (ii) wherein said sequence is less than 30 nucleotides in length.

5. The double-stranded ribonucleic acid molecule of claim 1, wherein said sense strand comprises nucleotide acid sequences depicted in SEQ ID Nos: 1, 3, 7, 9, 13, 15, 17, 19, 27 and 31 and said antisense strand comprises nucleic acid sequences depicted in SEQ ID Nos: 2, 4, 8, 10, 14, 16, 18, 20, 28 and 32 wherein said double-stranded ribonucleic acid molecule comprises the sequence pairs selected from the group consisting of SEQ ID NOs: 1/2, 3/4, 7/8, 9/10, 13/14, 15/16, 17/18, 19/20, 27/28, and 31/32.

6. The double-stranded ribonucleic acid molecule of claim 5, wherein the antisense strand further comprises a 3′ overhang of 1-5 nucleotides in length.

7. The double-stranded ribonucleic acid molecule of claim 6, wherein the overhang of the antisense strand comprises uracil or nucleotides which are complementary to the mRNA encoding IL-18.

8. The double-stranded ribonucleic acid molecule of claim 7, wherein the sense strand comprises a 3′ overhang of 1-5 nucleotides in length.

9. The double-stranded ribonucleic acid molecule of claim 8, wherein the overhang of the sense strand comprises uracil or nucleotides which are identical to the mRNA encoding IL-18.

10. The double-stranded ribonucleic acid molecule of claim 1, wherein said double-stranded ribonucleic acid molecule comprises at least one modified nucleotide.

11. The double-stranded ribonucleic acid molecule of claim 10, wherein said modified nucleotide is selected from the group consisting of a 2′-O-methyl modified nucleotide, a 5′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′ fluoro-modified nucleotide, a locked nucleotide, an abasic nucleotide, 2′-amino-modified nucleotide, 2′-alkyl-modified nucleotide, morpholino nucleotide, a phosphoramidate, a 5′-phosphate group at the 5′-terminal end of the antisense strand and a non-natural base comprising nucleotide.

12. The double-stranded ribonucleic acid molecule of any one of claim 11, wherein said modified nucleotide is a 2′-O-methyl modified nucleotide, a 5′-O-methyl modified nucleotide, a 2′ fluoro-modified nucleotide, a nucleotide comprising a 5′-phosphorothioate group deoxythymidine and 5′-phosphate group at the 5′-terminal end of the antisense strand.

13. The double-stranded ribonucleic acid molecule of claim 1, wherein the sense strand or antisense strand comprise an overhang of 1-2 deoxythymidines.

14. The double-stranded ribonucleic acid molecule of claim 1, wherein said double-stranded ribonucleic acid molecule comprises the sequence pairs selected from the group consisting of SEQ ID NOs: 149/150, 163/164, 165/166, 179/180, 183/184, 185/186, 187/188, 213/214, 215/216, 217/218, 239/240, 253/254, 255/256, 293/294, 435/436, 461/462, 463/464, 471/472, 475/476, 479/480 and 487/488.

15. A nucleic acid molecule comprising the sequence from the sense strand or antisense strand of the double-stranded ribonucleic acid molecule of claim 1.

16. A vector comprising a regulatory sequence operably linked to a nucleotide sequence that encodes at least the sense strand or antisense strand of the double-stranded ribonucleic acid molecule of claim 1.

17. A cell, tissue or non-human organism comprising the double-stranded ribonucleic acid molecule of claim 1.

18. A cell, tissue or non-human organism comprising the nucleic acid molecule of claim 15.

19. A cell, tissue or non-human organism comprising the vector of claim 16.

20. A pharmaceutical composition comprising the double-stranded ribonucleic acid molecule as defined in claim 1 and a pharmaceutically acceptable carrier, stabilizer and/or diluent.
